(19) United States
(12) Patent Application Publication
(10) Pub. No.: US 2024/0226887 A1
(43) Pub. Date: Jul. 11, 2024

(54) ANALYTE DETECTION CARTRIDGE AND METHODS OF USE THEREOF

(71) Applicants: Northwestern University, Evanston, IL (US); Minute Molecular Diagnostics, Inc., Evanston, IL (US)

(72) Inventors: Robin Jiwoong Kim, Evanston, IL (US); Abhishek k. Agarwal, Evanston, IL (US); Matthew Austin Butzler, Evanston, IL (US); David M. Kelso, Wilmette, IL (US); Sally M. McFall, Evanston, IL (US); Tom Westberg, Lake Forest, IL (US); Jennifer Lynn Reed, Evanston, IL (US); Kerry Berland, Evanston, IL (US)

(21) Appl. No.: 18/558,061

(22) PCT Filed: Apr. 27, 2022
(86) PCT No.: PCT/US22/26547

§ 371 Date: Oct. 30, 2023

Related U.S. Application Data
(60) Provisional application No. 63/304,034, filed on Jan. 28, 2022.
(60) Provisional application No. 63/289,481, filed on Dec. 14, 2021.
(60) Provisional application No. 63/274,332, filed on Nov. 1, 2021.
(60) Provisional application No. 63/180,270, filed on Apr. 27, 2021.

(51) Int. Cl. B01L 3/00; B01L 7/00; C12N 15/10; C12Q 1/6851; G01N 21/64

(52) U.S. Cl. CPC B01L 3/502723; B01L 7/52; C12N 15/1013; C12Q 1/6851; G01N 21/6428; B01L 2200/027; B01L 2200/04; B01L 2200/0684; B01L 2200/0689; B01L 2200/16; B01L 2300/0663; B01L 2300/1827; G01N 2021/6439

(57) ABSTRACT

Provided herein are devices (e.g., cartridges), instruments, systems, and components thereof for rapid sample processing and analyte detection (e.g., nucleic acid purification, amplification, and/or detection), and methods of use thereof. A cartridge for analyte detection may comprise: a storage section including a storage chamber; a processing section including a processing chamber; a microfluids section in fluid communication with the processing section; and a transfer capsule configured to transfer fluid between the storage chamber and the processing chamber

### CROSS-REFERENCE TO RELATED APPLICATIONS

This application claims the benefit of U.S. Provisional Patent Application No. 63/289,481, filed on Apr. 27, 2021; U.S. Provisional Patent Application No. 63/274,332, filed on Nov. 1, 2021; U.S. Provisional Patent Application No. 63/289,481, filed on Dec. 14, 2021; and U.S. Provisional Patent Application No. 63/304,034, filed on Jan. 28, 2022; each of which is incorporated by reference herein.

### STATEMENT REGARDING FEDERALLY SPONSORED RESEARCH

This invention was made with government support under grant number EB027049 awarded by the National Institutes of Health. The government has certain rights in the invention.

### FIELD

Provided herein are devices (e.g., cartridges), instruments, systems, and components thereof for rapid sample processing and analyte detection (e.g., nucleic acid purification, amplification, and/or detection), and methods of use thereof.

### BACKGROUND

Nucleic acid testing provides a method for the detection and diagnosis of infectious diseases among many other uses. The most widely practiced and most reliable methods of nucleic acid testing employ polymerase chain reaction (PCR). A limitation of PCR is that it requires an hour or more to cycle the reaction solution through multiple temperatures, which can differ by 30° C. or more. Quantitative or real-time PCR (qPCR or RT-PCR) takes even longer because fluorescence readings must be taken during or between each thermal cycle. The long processing time and electrical energy required to perform qPCR keep it from being used in many situations where a diagnosis must be made quickly and accurately.

A typical qPCR protocol performs 30-50 cycles of heating the test solution to 95° C., then cooling to 60° C., followed by fluorescence readings. In typical thermal cyclers, the heating and cooling steps are done in plastic tubes with a thermal electric cooler (TEC), which pumps heat in and out of the test solution through the walls of the tube. Such thermal cyclers introduce inefficiencies into the PCR procedures.

### SUMMARY

Provided herein are devices (e.g., cartridges), instruments, systems, and components thereof for rapid sample processing and analyte detection (e.g., nucleic acid purification, amplification, and/or detection), and methods of use thereof.

In some aspects, provided herein is a cartridge device and method for determining of levels of analytes with specific-binding methods (e.g., immunoassay, nucleic acid amplification). The analytes can be presented as bulk liquid solutions or absorbed in porous media such as swabs. The cartridge contains all the components and chambers needed to process and detect the target analyte. In other words, the cartridge is self-contained. The cartridge is acted on by a processing instrument with servomechanisms to perform operations including, but not limited to, heat transfer, liquid transfer, magnetic transfer, and optical detection.

In some embodiments, provided herein are cartridges for analyte detection comprising: a storage section including a storage chamber; a processing section including a processing chamber; a microfluids section in fluid communication with the processing section; and a transfer capsule configured to transfer fluid between the storage chamber and the processing chamber. In some embodiments, the processing section is positioned between the storage section and the microfluidic section. In some embodiments, cartridges further include a docking section with a access port in fluid communication with the storage chamber and a processing access port in fluid communication with the processing chamber. In some embodiments, cartridges further include a body that forms at least a portion of the storage section, at least a portion of the processing section, and at least a portion of the docking section. In some embodiments, a first channel fluidly connects the storage access port and the storage chamber, and a second channel fluidly connects the processing access port and the processing chamber. In some embodiments, the storage chamber includes a first end and a second end opposite the first end, the first end is positioned closer to the storage access port than the second end, and wherein the first channel connects to the storage chamber at the second end. In some embodiments, the processing chamber includes a first end and second end opposite the first end, the first end is positioned closer to the processing access port than the second end, and wherein the second channel connects to the processing chamber at the second end. In some embodiments, the storage chamber is a first storage chamber and the storage section further includes a second storage chamber. In some embodiments, the processing chamber is a first processing chamber and the processing section further includes a second processing chamber. In some embodiments, the storage section includes a cavity configured to receive the transfer capsule. In some embodiments, cartridges further include a first vent fluidly coupled to the storage chamber, and a second vent fluidly coupled to the processing chamber. In some embodiments, the microfluids section includes a reaction chamber, a microfluidic vent channel fluidly connected to the reaction chamber, and a microfluidic inlet channel fluidly connecting the processing chamber and the reaction chamber. In some embodiments, the microfluids section further includes a wax seal. In some embodiments, the wax seal is a first wax seal and the microfluids section further includes a second wax seal, wherein the first wax seal is positioned adjacent the microfluidic inlet channel and the second wax seal is positioned adjacent the microfluidic vent channel. In some embodiments, the second wax seal is positioned a distance from the reaction chamber, wherein the distance is at least 2 mm. In some embodiments, cartridges further include an offset vent channel fluidly connected to the microfluidic inlet channel. In some embodiments, the offset vent channel is a first offset vent channel and the cartridge further includes a second offset vent channel fluidly connecting the first offset vent channel and the second vent.

In some embodiments, provided herein are microfluidic devices comprising: (i) a reaction chamber, (ii) an inlet channel in fluid communication with the reaction chamber; (iii) a vent channel in fluid communication with the reaction chamber; (iv) a first wax seal positioned adjacent to and in fluid communication with the inlet channel, wherein when first the wax seal is in a first position it does not occlude the inlet channel and allows fluid to enter the reaction chamber through the inlet channel, and wherein when first the wax seal is in a second position the first wax seal occludes the inlet channel and prevents fluid from entering or escaping the reaction chamber through the inlet channel; (v) a second wax seal positioned adjacent to and in fluid communication with the vent channel, wherein when second the wax seal is in a first position it does not occlude the vent channel and allows gas to exit the reaction chamber through the vent channel, and wherein when second the wax seal is in a second position the second wax seal occludes the vent channel and prevents fluid from entering or escaping the reaction chamber through the vent channel; wherein liquid reagents can be introduced to the reaction chamber through the inlet channel; and wherein heating the wax seals above a threshold temperature melts the wax seals and subsequently cooling the wax seals below the threshold temperature solidifies the wax seals in the second position.

In some embodiments, provided herein are systems comprising a cartridge described herein and an instrument into which the cartridge can be inserted, wherein the instrument comprises components to impart heating, magnetic transfer, fluid transfer, and/or analyte detection functionalities onto the cartridge. In some embodiments, provided herein is the use of the systems herein for sample processing and analyte detection.

In some embodiments, provided herein is a microfluidic system comprising an inlet channel, a vent channel, and a reaction chamber; wherein the inlet channel is in fluid communication with the reaction chamber, wherein the vent channel is in fluid communication with the reaction chamber; the system further comprising a heating element capable of raising the temperature of the reaction chamber; the system further comprising a first seal positioned adjacent to and in fluid communication with the inlet channel, wherein when first the seal is in a first position it does not occlude the inlet channel and allows fluid to enter the reaction chamber through the inlet channel, and wherein when the first seal is in a second position the first seal occludes the inlet channel and prevents fluid from entering or escaping the reaction chamber through the inlet channel; the system further comprising a second seal positioned adjacent to and in fluid communication with the vent channel, wherein when the second seal is in a first position it does not occlude the vent channel and allows gas to exit the reaction chamber through the vent channel, and wherein when the second seal is in a second position the second seal occludes the vent channel and prevents fluid from entering or escaping the reaction chamber through the vent channel; and wherein heating the seals above a threshold temperature melts the seals and allows the seals to flow from the first positions into the second positions, and wherein subsequently cooling the seals below the threshold temperature solidifies the wax seals in the second position. In some embodiments, the first seal and the second seal comprise a wax or polymeric material.

In some embodiments, provided herein are heat transfer devices for heating and cooling a reaction chamber, the heat transfer device comprising: a heat reservoir including a base, a first bore, a second bore, and a heat exchanger extending from the base; the heat exchanger includes a planar surface configured to about the reaction chamber; a heater positioned within the first bore; and a temperature sensor positioned within the second bore. In some embodiments, the first bore and the second bore are formed in the base. In some embodiments, the heat exchanger is cylindrical. In some embodiments, the heat reservoir is Aluminum. In some embodiments, the heater is an electric resistive heater. In some embodiments, the reaction chamber is a PCR reaction chamber. In some embodiments, devices further comprise a processor and a non-transitory memory including instructions that when executed by the processor performs closed-loop temperature control of the heat reservoir.

In some embodiments, provided herein are assemblies comprising: a first support; a second support movable with respect to the first support; a first heat transfer device with a first planar surface, the first heat transfer device coupled to the first support; a second heat transfer device with a second planar surface positioned opposite the first planar surface, the second heat transfer device coupled to the second support; wherein the first heat transfer device and the second heat transfer device are at a first temperature; a third heat transfer device with a third planar surface, the third heat transfer device coupled to the first support; a fourth heat transfer device with a fourth planar surface positioned opposite the third planar surface, the fourth heat transfer device coupled to the second support; wherein the third heat transfer device and the fourth heat transfer device are at a second temperature different than the first temperature. In some embodiments, assemblies further include a fluorimeter coupled to the first support and positioned between the first heat transfer device and the third heat transfer device. In some embodiments, assemblies further include a fifth heat transfer device with a fifth planar surface position opposite the fluorimeter, the fifth heat transfer device coupled to the second support and positioned between the second heat transfer device and the fourth heat transfer device. In some embodiments, the assembly is configured to receive a reaction chamber between the first planar surface and the second planar surface to bring the reaction chamber to the first temperature; and between the third planar surface and the fourth planar surface to bring the reaction chamber to the second temperature. In some embodiments, the reaction chamber is a PCR chamber. In some embodiments, assemblies further comprise an actuator coupled to the second support and configured to move the second support along a clamp axis between a first position in which the first planar surface and the second planar surface are spaced apart by a first distance, and a second position in which the first planar surface and the second planar surface are spaced apart by a second distance, smaller than the first distance. In some embodiments, the second distance is within a range of 400 micrometers to 600 micrometers. In some embodiments, the actuator is a first actuator, and the assembly further includes a second actuator coupled to the first support and the second support, wherein the second actuator is configured to move the first support and the second support together along a translation axis. In some embodiments, the translation axis is normal to the clamp axis. In some embodiments, the first temperature is within a range of 80 degrees C. and 100 degrees C. In some embodiments, the second temperature is within a range of 50 degrees C. and 70 degrees C.

In some embodiments, provided herein are fluidic devices comprising: a reaction chamber; a channel in fluid communication with the reaction chamber; and a wax seal, wherein when the wax seal is in a first position it does not occlude the channel and allows fluid to enter or exit the reaction chamber through the channel, and when the wax seal is in a second position the wax seal occludes the channel and prevents fluid from entering or escaping the reaction chamber through the channel; wherein heating the wax seal above a threshold temperature melts the wax seal and subsequently cooling the wax seal below the threshold temperature solidifies the wax seals in the second position. In some embodiments, the reaction chamber is in fluid communication with an inlet channel; and the fluidic device further includes a vent channel in fluid communication with the reaction chamber. In some embodiments, the inlet channel wax seal is a first wax seal and the fluidic device further includes a second wax seal, wherein when second the wax seal is in a first position it does not occlude the vent channel and allows fluid to exit the reaction chamber through the vent channel, and when the second wax seal is in a second position the second wax seal occludes the vent channel and prevents fluid from entering or escaping the reaction chamber through the vent channel. In some embodiments, the first wax seal is positioned adjacent the inlet channel and the second wax seal is positioned adjacent the vent channel. In some embodiments, fluidic devices further comprise a first high-temperature movable heater capable of being positioned within a range of the wax seal to heat the wax seal above the threshold temperature. In some embodiments, fluidic devices further comprise a second low-temperature movable heater capable of being positioned within a range of the wax seal to cool the wax seal below the threshold temperature. In some embodiments, the diameter of the wax seal is less than the diameter of the first and second heaters. In some embodiments, the wax seal is coated in an adhesive. In some embodiments, the adhesive is an acrylic adhesive. In some embodiments, the first and second heaters are capable of being positioned at selected positions relative to the wax seal. In some embodiments, the second wax seal is positioned a distance from the reaction chamber, wherein the distance is at least 2 mm. In some embodiments, a plurality of liquid reagents can be introduced to the reaction chamber through the inlet channel.

In some embodiments, provided herein are fluorimeters comprising: a casing including a measurement aperture; a first light source coupled to the casing along a first light source axis; a second light source coupled to the casing along a second light source axis; a third light source coupled to the casing along a third light source axis; a fourth light source coupled to the casing along a fourth light source axis; a first light detector coupled to the casing along a first detector axis; a second light detector coupled to the casing along a second detector axis; a third light detector coupled to the casing along a third detector axis; a fourth light detector coupled to the casing along a fourth detector axis; wherein the first light source axis, the second light source axis, the third light source axis, the fourth light source axis, the first detector axis, the second detector axis, the third detector axis, and the fourth detector axis intersect the measurement aperture. In some embodiments, the circular measurement aperture defines a normal axis through its center and perpendicular to the plane of the measurement aperture. In some embodiments, the normal axis, the first light source axis, the second light source axis, the third light source axis, the fourth light source axis, the first detector axis, the second detector axis, the third detector axis, and the fourth detector axis are not co-axial. In some embodiments, the first light source axis, the second light source axis, the third light source axis, the fourth light source axis, the first detector axis, the second detector axis, the third detector axis, and the fourth detector axis are positioned circumferentially around the normal axis. In some embodiments, the first light source is positioned circumferentially adjacent to the first detector. In some embodiments, the fluorimeter does not include a dichroic mirror or a beam splitter. In some embodiments, fluorimeters further comprising a processor and a non-transitory memory including instructions that, when executed by the processor, store 400 analog to digital readings by the first detector made over a 100 millisecond time period. In some embodiments, the first light source emits a first excitation light along the first light source axis; and wherein the first excitation light is reflected at the measurement aperture away from the first light detector axis. In some embodiments, the measurement aperture is configured to receive a sample; and wherein the first excitation light from the first light source has a first spectrum and the first light detector measures a first fluorescence of the sample in response to the first excitation light. In some embodiments, a second excitation light from the second light source has a second spectrum and the second light detector measures a second fluorescence of the sample in response to the second excitation light. In some embodiments, the measurement aperture is configured to align with a planar surface of a PCR chamber. In some embodiments, the first detector includes a first lens, a filter, a second lens, and a solid-state detector.

In some embodiments, provided herein are nucleic acid quantification methods comprising: (a) performing a multicycle amplification reaction on a sample suspected of containing a target nucleic acid in the presence of a detectable reporter to produce an amplification product; (b) detecting a signal from the detectable reporter that correlates with the amount of detectable reporter incorporated into the amplification product after each cycle of the amplification reaction; (c) identifying earliest cycle with a normalized increase in signal that is greater than a cutoff value; (d) fitting a linear equation to a plurality of signals from cycles earlier than the earliest cycle with a normalized increase in signal that is greater than the threshold value; (e) fitting a curve to a plurality of signals from cycles later than the earliest cycle with a normalized increase in signal that is greater than the threshold value; (f) identifying the cycle (Cq) for which the normalized difference in signal for the linear equation and the curve is equal to a threshold value; wherein Cq is inversely proportional to the amount of target nucleic acid present in the sample. In some embodiments, step (c) comprises: (i) identifying the cycle with the maximum normalized increase in signal; (ii) if the maximum normalized increase in signal is greater than the cutoff value, then determine the earliest cycle prior to the cycle with the maximum normalized increase in signal that has a normalized increase in signal that is greater than a lower cutoff value. In some embodiments, methods further comprise a step of calculating a moving average of the detected signal for each cycle of the amplification reaction and using the moving averages for each cycle for steps (c)-(f). In some embodiments, the moving average is calculated as the average of the signal at each cycle with signals at the immediate two earlier and immediate two later cycles. In some embodiments, the curve is a quadratic curve. In some embodiments, the multicycle amplification reaction is a 30-50 (e.g., 35, 40, 45) cycle amplification reaction. In some embodiments, the multicycle amplification reaction is a 40 cycle amplification reaction. In some embodiments, the multicycle amplification reaction is quantitative polymerase chain reaction (qPCR). In some embodiments, the detectable reporter is a fluorophore and the signal is fluorescence. In some embodiments, each cycle comprises a nucleic acid denaturation step, an annealing/extension step, and a detection step. In some embodiments, each cycle comprises a nucleic acid denaturation step, an annealing step, an extension step, and a detection step. In some embodiments, the sample is a biological sample. In some embodiments, the target nucleic acid is a viral nucleic acid. In some embodiments, the amount of target nucleic acid present in the sample is proportional to the viral load in the sample.

In some embodiments, provided herein are kits and methods for the rapid isolation of target nucleic acids from a biological sample. In particular, reagents and method steps are provided for the release and capture of target nucleic acids from whole cells, the isolation of target nucleic acids from cellular contaminants, and the amplification/detection of target nucleic acids.

In some embodiments, provided herein are methods and kits for preparing specimens for PCR analysis that reduce the time needed to lyse cells, extract nucleic acids, capture target nucleic acids, and separate them from contaminating/interfering substances. The methods/kits herein find use in a variety of different platforms, including but not limited to, manual manipulation using pipettes and tubes, automation using a self-contained single-use cartridge and external operating instrument, or automation using a robotic high-throughput platform and microwell plates.

The methods/kits herein reduce the complexity of nucleic acid purification and detection. In some embodiments, the methods/kits herein comprise a liquid reagent (e.g., a single buffer composition that is employed at multiple steps of the processes herein). In some embodiments, the methods/kits herein comprise two more liquid reagents. In some embodiments, the liquid reagent is used to bring the biological sample into suspension in a volume sufficient to manipulate in the process steps herein, and to resuspend multiple dry reagents (e.g., lyophilized reagents). In some embodiments, the dry reagents include a lysis reagent (e.g., proteinase K, SDS, and salt), a capture reagent comprising nucleic acid probes (e.g., nucleic acids comprising hybridization sequences tethered to a capture moiety (e.g., biotin)), and capture-agent-coated magnetic beads (e.g., streptavidin-coated paramagnetic beads). In some embodiments, an analysis reagent, for example, containing components necessary to amplify and detect/quantify a target nucleic acid, is provided.

Features of embodiments of the present technology include, but are not limited to, one or more of the following: changing the chemical formulation of the working suspension by only adding reagents, without removing fractions by precipitation, centrifugation, or filtration (prior to the magnetic separation step); use of the same aqueous buffer for multiple steps (e.g., lysis, wash, resuspension, and/or amplification/detection); actively mixing solutions and suspensions by vigorously transferring reactants in and out of reaction vessels; transferring solutions and suspensions between temperature-controlled chambers with optimized temperatures that increase rates and efficiencies of reactions; rapidly heating specimen solution to obtain temperatures for breakdown of proteins, cell lysis and PK denaturation; no pausing at optimum temperature for PK activity; delayed addition of streptavidin coated magnetic beads to improve efficiency of binding to solid phase by giving biotinylated probes time to hybridize with targets in solution; pelleting/capturing magnetic beads by placing magnet near the inlet to a channel or pipette; resuspending pelleted beads by lifting them off the film with an oscillating air-water interface; amplifying target nucleic acids while bound to magnetic beads via a capture probe; directly rehydrating dry/concentrated amplification reagents with a magnetic bead suspension; binding target-probe complex to beads with biotin-avidin so it can be unbound at lower temperatures that denature avidin; etc. The oscillating air-water interface is created by pumping a given volume of buffer in and out of the chamber.

Various devices, instruments, systems, components thereof, reagents, and methods are described herein and may find use in embodiments together or independently. For example, reagents described herein may find use in performing methods described herein using a system described herein (e.g., cartridge and instrument). Alternatively, the methods herein may be performed independently from the cartridges and instruments herein. Similarly, components of the cartridges and instruments described herein may find use in other devices, in the performance of methods without the cartridges or instruments described herein, and/or in applications not specifically addressed herein. Various combinations are contemplated of the method steps, compositions, and/or components described herein, and such combinations are within the scope herein. Similarly, the method steps, compositions, and/or components described herein may find use independently of the methods, systems, devices, and systems described herein.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (“FIG.”) relating to one or more embodiments.

The patent or application file contains at least one drawing executed in color. Copies of this patent or patent application publication with color drawing(s) will be provided by the Office upon request and payment of the necessary fee.

FIG. 1 is a perspective view of a cartridge according to one embodiment.

FIG. 2 is a perspective view of the cartridge of FIG. 1, with portions removed for clarity and a transfer chamber in a storage configuration.

FIG. 3 is an exploded front view of the cartridge of FIG. 1.

FIG. 4 is an exploded rear view of the cartridge of FIG. 1.

FIG. 5 is a perspective view of a body of the cartridge of FIG. 1.

FIG. 6 is a partial cross-sectional view of a storage section of the cartridge of FIG. 1.

FIG. 7 is a partial cross-sectional view of a processing section of the cartridge of FIG. 1.

FIG. 8 is a cross-sectional view of the cartridge of FIG. 1 with the transfer chamber fluidly coupled to the processing section.

FIG. 9 is a partial cross-sectional view of a microfluid section of the cartridge of FIG. 1.

FIG. 10A is a side view of the microfluid section with wax seals in an initial configuration.

FIG. 10B is a side view of the microfluid section with wax seals in a sealed configuration.

FIG. 11 is a partial perspective view of the microfluid section.

FIG. 12 is a cross-sectional view of the microfluid section.

FIG. 13 is a side view of a microfluid section according to another embodiment.

FIG. 14 is a side view of a microfluid section according to another embodiment.

FIGS. 15A and 15B are views of a cartridge in which each access port for the various chambers is depicted with overmolded seals (blue).

FIG. 16 contains view of a cartridge comprising energy directors (green) for use in heat sealing components of cartridge.

FIG. 17 is a perspective view of a heating and detecting assembly.

FIG. 18 is a side view of a system including a cartridge and the heating and detecting assembly of FIG. 17.

FIG. 19 is a perspective view of a portion of the heating and detecting assembly of FIG. 17.

FIG. 20 is a side view of FIG. 19.

FIG. 21 is a perspective view of a heat transfer device.

FIG. 22 is a perspective view of a heat transfer device.

FIG. 23 is a perspective cross-sectional view of the heat transfer device of FIG. 22.

FIG. 24 is a graph of temperature versus time for a reaction chamber of the cartridge acted upon by the heating and detecting assembly of FIG. 18.

FIG. 25 is an enlarged portion of the graph of FIG. 24.

FIG. 26 is a partial cross-sectional view of a microfluid section of the cartridge of FIG. 18.

FIG. 27A is a side view of the microfluid section with wax seals in an initial configuration.

FIG. 27B is a side view of the microfluid section with wax seals in a sealed configuration.

FIG. 28 is a top view of a detector portion of the heating and detecting assembly of FIG. 17.

FIG. 29 is a partial cross-sectional view of the heating and detecting assembly of FIG. 17.

FIG. 30 is a partial cross-sectional view of the heating and detecting assembly of FIG. 17.

FIG. 31 is a perspective view of a portion of a fluorimeter, with portions removed for clarity.

FIG. 32 is a partial cross-sectional view of the fluorimeter of FIG. 31.

FIG. 33 is an example graph of fluorescence readings of a positive SARS-CoV-2 specimen.

FIG. 34 is a view of the region surrounding the breakpoint of the graph depicted in FIG. 33.

### DETAILED DESCRIPTION

Provided herein are devices (e.g., cartridges), instruments, systems, and components thereof for rapid sample processing and analyte detection (e.g., nucleic acid purification, amplification, and/or detection), and methods of use thereof.

In some embodiments, provided herein is a cartridge device that is useful for detecting and/or quantitating levels of analytes (e.g., via immunoassay, nucleic acid amplification). The analytes can be present in liquid samples or absorbed in porous media such as swabs. The cartridge contains all the components (e.g., reagents, buffers, beads, etc.) and chambers needed to process and detect the target analyte. In other words, the cartridge is self-contained. The cartridge is acted on by a processing instrument with servomechanisms to perform operations including, but not limited to, heat transfer, liquid transfer, magnetic transfer, and optical detection. In some embodiments, the cartridge is acted on by an adjustable assembly with a plurality of heat transfer devices mounted thereon. In some embodiments, the adjustable assembly further includes a fluorimeter integrated in and amongst the plurality of heat transfer devices. In some embodiments, reagents and method steps are provided for the release and capture of target nucleic acids from whole cells, the isolation of target nucleic acids from cellular contaminants, and the amplification/detection of target nucleic acids.

### I. Definitions

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “subject” and “patient” refer to any animal, such as a dog, cat, bird, livestock, and particularly a mammal, preferably a human.

As used herein, the term “sample” and “specimen” are used interchangeably, and in the broadest senses. In one sense, sample is meant to include a specimen or culture obtained from any source, as well as biological and environmental samples. Biological samples may be obtained from animals (including humans) and encompass fluids, solids, tissues, and gases. Biological samples include blood products, such as plasma, serum, stool, urine, and the like. Environmental samples include environmental material such as surface matter, soil, mud, sludge, biofilms, water, and industrial samples. Such examples are not however to be construed as limiting the sample types applicable to the present invention.

As used herein, the term “lysate” refers to the solution and/or suspension that results from the lysis (breaking open) of cells and/or viruses to release the nucleic acids contained therein. The term “whole lysate” refers to a lysate that contains all of the component parts of the original sample, without removal of any components. The terms “partial lysate” or “purified lysate” refer to a lysate that has have one or more components or fractions removed therefrom.

The term “system” as used herein refers to a collection of compositions, devices, articles, materials, etc. (e.g., a cartridge and an instrument) grouped together in any suitable manner (e.g., physically associated; in fluid-, electronic-, or data-communication; packaged together; etc.) for a particular purpose.

The term “cartridge” refers to a device that comprises, for example a plurality of chamber, compartments, microfluidics, channels, etc. for containing/mixing reagents and samples, but does not contain all the necessary fluid handling mechanisms, magnetic particle handling mechanisms, heaters, etc. to function independently of a separate instrument containing such mechanisms. When a cartridge interfaces with the instrument (e.g., when the cartridge is placed/inserted into the instrument), the mechanisms within the instrument appropriately align with the cartridge to provide the necessary functionality. A cartridge may, in certain embodiments, be designed for a single use, after which it is discarded. In other embodiments, a cartridge is provided for multiple use. In certain embodiments, one or more of the compartments in a cartridge contains a reagent.

The term “system” as used herein refers to a collection of compositions, devices, articles, materials, etc. (e.g., a cartridge and an instrument) grouped together in any suitable manner (e.g., physically associated; in fluid-, electronic-, or data-communication; packaged together; etc.) for a particular purpose.

As used herein, the term “preparing” and linguistic equivalents thereof refers to any steps taken to alter a sample or one or more components thereof, for example, for use in a subsequence analysis or detection step. Exemplary sample preparation steps include, for example, dilution or concentration of a sample, isolation or purification of a sample component, heating or cooling a sample, amplification of a sample component (e.g., nucleic acid), labeling a sample component, etc.

As used herein, the term “analyzing”, and linguistic equivalents thereof refers to any steps taken to a characterize a sample or one or more components thereof. Exemplary analysis steps include, for example, quantification of a sample component (e.g., a target nucleic acid), sequencing a sample component, etc.

As used herein, the term “processor” (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor is a microprocessor that can be configured to communicate in a stand-alone and/or a distributed environment, and can be configured to communicate via wired or wireless communications with other processors, where such one or more processors can be configured to operate on one or more processor-controlled devices that can be similar or different devices.

As used herein, the term “memory” is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processor can be connected to the memory and execute software instructions that are capable of being stored in a RAM of the memory (e.g., during execution), a ROM of the memory (e.g., on a generally permanent bases), or another non-transitory computer readable medium such as another memory or a disc. In some embodiments, the memory includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor can be configured to retrieve from the memory and execute, among other things, instructions related to the processes and methods described herein.

### II. Cartridge

In some embodiments, provided herein are devices for rapid sample processing and analyte detection. In some embodiments, devices find use in the processing of any suitable samples (e.g., biological sample (e.g., solid sample (e.g., tissue biopsy), liquid sample (e.g., blood, saliva, urine, etc.)), environmental sample, etc.). In particular embodiments, devices herein find use in processing samples containing cells and detecting/quantitating cellular analytes (e.g., nucleic acids, antigens, etc.).

In some embodiments, the devices herein comprise cartridges that interface with (e.g., are inserted into) a complementary instrument. In some embodiments, the cartridge devices herein comprise the chambers, channels, vents, ports, reagents (e.g., lysis reagents, digestion reagents, capture probes, primers, paramagnetic particles (PMPs) necessary for sample processing and analyte detection. However, the cartridge lacks the heating elements, pressure source, magnetic transfer mechanism, fluorescence detection mechanism, etc. necessary for sample processing and analyte detection. The complementary instrument comprises the mechanisms and functionalities necessary for sample processing and analyte detection that are lacking from the cartridge. For example, in some embodiments, the complementary instrument includes an adjustable heating and detecting assembly with a plurality of heat transfer devices and a fluorimeter. In some embodiments, the complementary instrument includes a magnetic transfer element, for example, for moving magnetic materials within a cartridge associated therewith. In some embodiments, the complementary instrument includes a pressure element, for example, withdrawing and depositing fluids into and/or out of a cartridge (or chamber thereof). In some embodiments, a cartridge is disposable (e.g., single use). In other embodiments, the cartridge is a multiple-use device, but must be cleaned and/or reloaded with reagents between uses. Cartridges are used with a multi-use instrument. In some embodiments, the instrument finds use with multiple different cartridges for performing different sample processing and/or detection assays/protocols. In some embodiments, an instrument is specific for a single cartridge configuration.

In some embodiments, the cartridge device comprises two or more chambers for containing a volume of liquid (e.g., 0.1 ml, 0.2 ml, 0.5 ml, 1.0 ml, 1.5 ml, 2.0 ml, or more). In some embodiments, one or more of the chambers comprises an access channel that runs from the bottom of the chamber (e.g., the side of the bottom of the chamber) upwards toward the top of the device, terminating at an access port. The access channels allow liquid within the chambers to be withdrawn through the access channel by applying negative pressure at the access port. Because the inlet of the access channel to the chamber is at the bottom of the chamber (e.g., side of the bottom), liquid added to the chamber immediately pools at the bottom of the chamber, and approximately 100% (e.g., 99.9%, 99.5%, 99%, 98.5%, 98%, 95%, or ranges therebetween) of the liquid within the chamber can be removed via the access channel. In some embodiments, one or more (e.g., all) of the access chambers comprise a vent channel. The vent channel allows the chamber to remain at ambient atmospheric pressure while pressure is applied to the access channel, thereby allowing liquid to be moved in/out of the access channel.

In some embodiments, the cartridge device comprises a transfer capsule. In some embodiments, a transfer capsule comprises an open top at one end, an open tip, and an open lumen running therebetween. In some embodiments, the transfer capsule resembles a pipette tip. In some embodiments, a transfer capsule is stored within a cavity in the device (e.g., a cavity that sits adjacent to and aligned with the chambers of the device (e.g., within the storage section of the device). In some embodiments, the open top of the transfer capsule is configured to interface with a nozzle on a pressure element of the complementary instrument (e.g., in the same manner as a pipette tip interfacing with a pipette). In some embodiments, the pressure element engages with the transfer capsule, is able to lift the transfer capsule up and out of the storage cavity, and it able to move the transfer capsule laterally along the storage and processing sections of the device. In some embodiments, the tip of the transfer capsule is configured to interface with (e.g., sit within, form a seal with, etc.) the access ports linked to the chambers. In some embodiments, the pressure source applies negative pressure, to draw liquid up from the chamber, through the access channel, and into the transfer capsule, via the access port. In some embodiments, the pressure source applies positive pressure, to eject liquid from the transfer capsule, through the access port and access channel, and into the chamber. In some embodiments, repeated cycles of positive and negative pressure from the pressure source are used to mix samples within the chamber (e.g., drawing the liquid into and out of the chamber and transfer capsule). In some embodiments, the pressure source and transfer capsule are used to move liquid between chambers. Because the transfer capsule can be moved into alignment with the access ports for multiple chambers, the system can be used to move liquid between any of those multiple chambers. This is in contrast to other devices that link chambers by channels and are therefore limited by the connectivity of the chambers or require valves to open/shut fluid communication between the various chambers.

In some embodiments, while the processing and storage chambers are accessed via the transfer capsule, the detection or reaction chamber(s) are accessed by microfluidics. In some embodiments, the final chamber of the processing section is accessible both by the transfer capsule (through an access channel and port) and by a microfluidic channel that runs from the processing chamber to a detection or reaction chamber. In some embodiments, fluids added to the final processing chamber will flow through the microfluidics to the detection or reaction chamber. In some embodiments, pelleted PMPs in the final processing chamber can be transferred, using the magnetic transfer element of the complementary instrument, through the microfluidics and into the detection or reaction chamber. Exemplary configurations for connecting the processing and microfluidic sections of the devices herein are described herein.

In some embodiments, for reaction (e.g., PCR) and/or detection (e.g., fluorescence detection) to accurately be conducted, the detection or reaction chamber is sealed, to prevent material introduction or escape during reaction or detection. In some embodiments, the devices herein allow for sealing of the reaction/detection chamber without valves or the like. In some embodiments, a wax seal resides adjacent to the inlet channel for the reaction/detection chamber. In its first position, the first wax seal does not occlude fluid access to the reaction/detection chamber via the inlet channel. Similarly, in some embodiments, a second wax seal resides adjacent to the vent channel for the reaction/detection chamber. In its first position, the second wax seal does not occlude air flow from the reaction/detection chamber. However, if sufficient heat is applied to the wax seals (e.g., via heater in the complementary instrument located adjacent and in close proximity to the reaction/detection section of the cartridge device), the wax seals melt and flow into the inlet channel and vent channel. If allowed to cool in the inlet channel and vent channel, the wax forms seals in the respective channels, preventing the flow of liquids or gas to/from the reaction/detection chamber.

In some embodiments, the direction of melted wax flow from a chamber, for example, when two or more channels are in fluid communication with the chamber, can be influenced by selectively placing the heater in the direction opposite the desired flow path. In such embodiments, the heater will melt wax that moves opposite the desired flow path, but allow wax moving in the desired direction to solidify in the desired channel more readily, thereby allowing the direction of flow of the melted wax to be influenced.

In some embodiments, sample preparation steps and analysis steps take place simultaneously and/or are repeated in series. For example, in qPCR, nucleic acid amplification and quantification steps are repeated in succession.

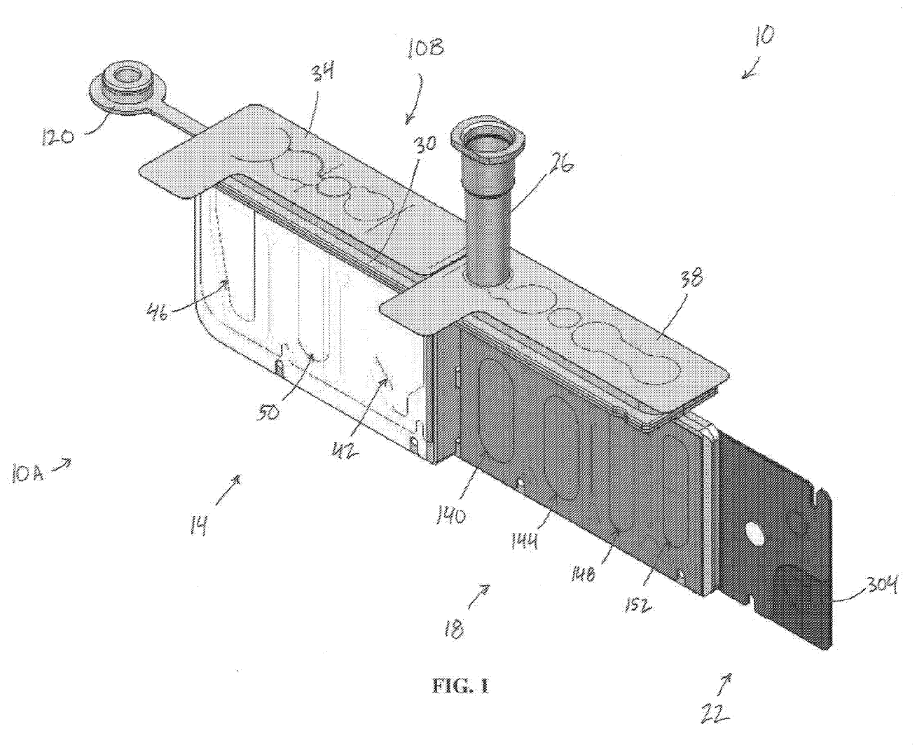

An exemplary cartridge device, containing elements described herein and capable of performing the various functions described herein in depicted in, for example, FIGS. 1-16. Other cartridges comprising different combinations and configurations of the elements depicted in FIGS. 1-16 are within the scope herein.

With reference to FIG. 1, a cartridge 10 for detecting levels of an analyte is illustrated. The cartridge 10 includes a storage section 14, a processing section 18, and a microfluidic section 22. In the illustrated embodiment, the processing section 18 is positioned between the storage section 14 and the microfluidic section 22. The cartridge 10 contains all the components and chambers needed to process and detect a target analyte. In some embodiments, the cartridge 10 is acted on by a processing apparatus that includes a servomechanism, for example, to perform operations including, but not limited to, heat transfer, liquid transfer, magnetic transfer, and optical detection.

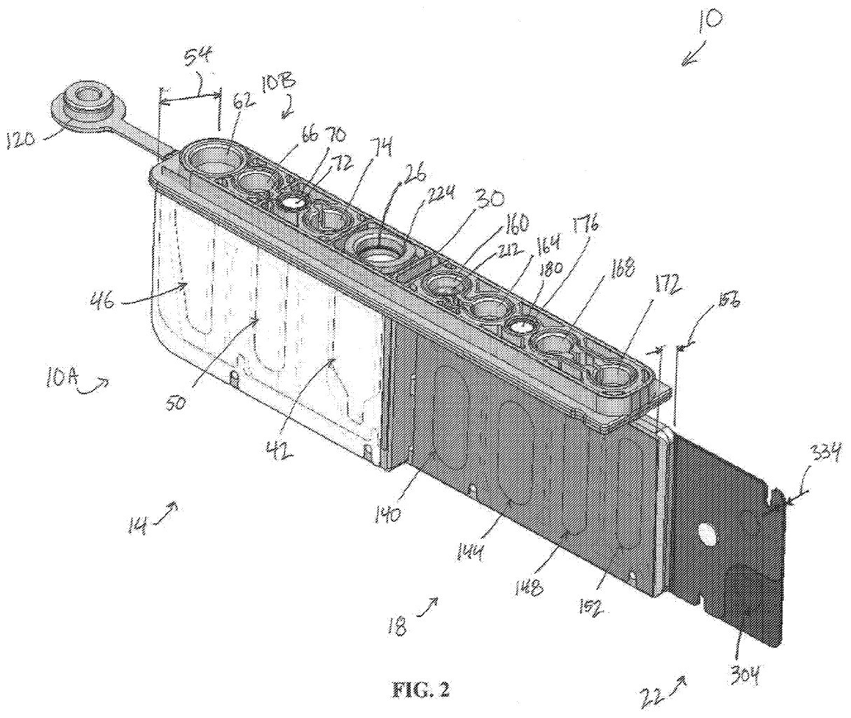

In general, the storage section 14 contains the specimen to be analyzed along with buffers and a transfer capsule 26 used in processing; the processing section 18 is where targets are extracted, purified and bound to magnetic beads; and the microfluidic section 22 is where the target or targets are detected. In the illustrated embodiment of FIG. 2, a connector 30 (or docking section) is coupled to the storage section 14 and the processing section 18. The connector 30 (or docking section) provides an interface where a specimen is loaded and where liquids are transferred between chambers. In other words, the connector 30 (or docking section) provides, among other things, access to fluid contained within the storage section 14 and processing section 18.

With continued reference to FIG. 1, the cartridge 10 includes a first seal 34 coupled to a portion of the connector 30 (or docking section) corresponding to the storage section 14, and a second seal 38 coupled to a portion of the connector 30 (or docking section) corresponding to the processing section 18. In some embodiments, the first seal 34 and the second seal 38 are heat sealed foil lidstocks. The transfer capsule 26 is illustrated positioned outside of the storage section 14 in FIG. 1. However, in some embodiments, the transfer capsule 26 is positioned within a cavity 42 formed in the storage section 14 (see FIG. 2).

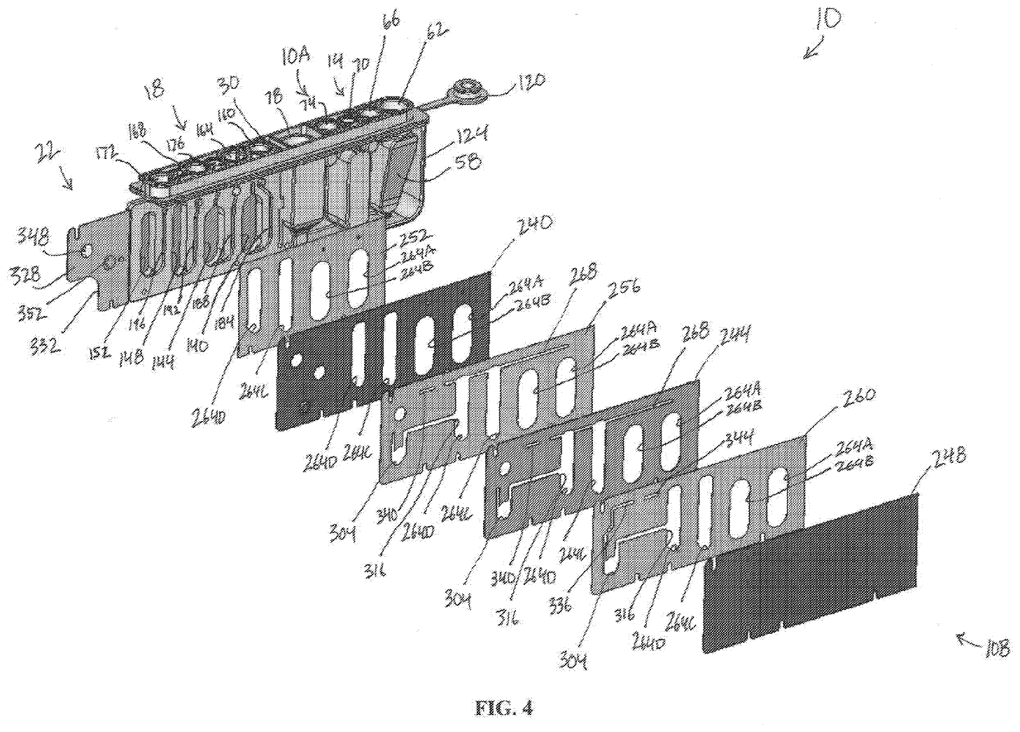

With reference to FIG. 2, the cartridge 10 is illustrated with the first seal 34 and the second seal 38 removed for clarity and with the transfer capsule 26 positioned within the cavity 42. In the illustrated embodiment, the storage section 14 includes a first chamber 46 (e.g., for containing a test specimen) and a second chamber 50 (e.g., for containing buffer). In other embodiments, the storage section 14 includes more or less than the two chambers. For example, in some embodiments, the storage section 14 includes a single chamber. In other embodiments the storage section 14 includes at least three chambers (e.g., 3, 4, 5, 6, or more). In the illustrated embodiment, the storage section 14 defines a width 54 that is greater than 3 mm (width 156 in section 18 is 3 mm). In some embodiments, the width 54 is greater than 3 mm (e.g., 4 mm, 5 mm, 6 mm, 7 mm, 8 mm, 9 mm, 10 mm, or more or ranges therebetween). In the illustrated embodiment, the first and second chambers 46, 50 each hold volumes of more than approximately 1 ml, although other size storage chambers are contemplated (e.g., 0.5 ml, 0.75 ml, 1.25 ml, 1.5 ml, 1.75 ml, 2.0 ml, or more, or ranges therebetween). In some embodiments, the first chamber 46 is formed with a slanted wall 58 (FIG. 4) that is shaped such that all but a few microliters can be transferred to the processing section 18. In other words, the slanted wall 58 at least partially forms the first chamber 46.

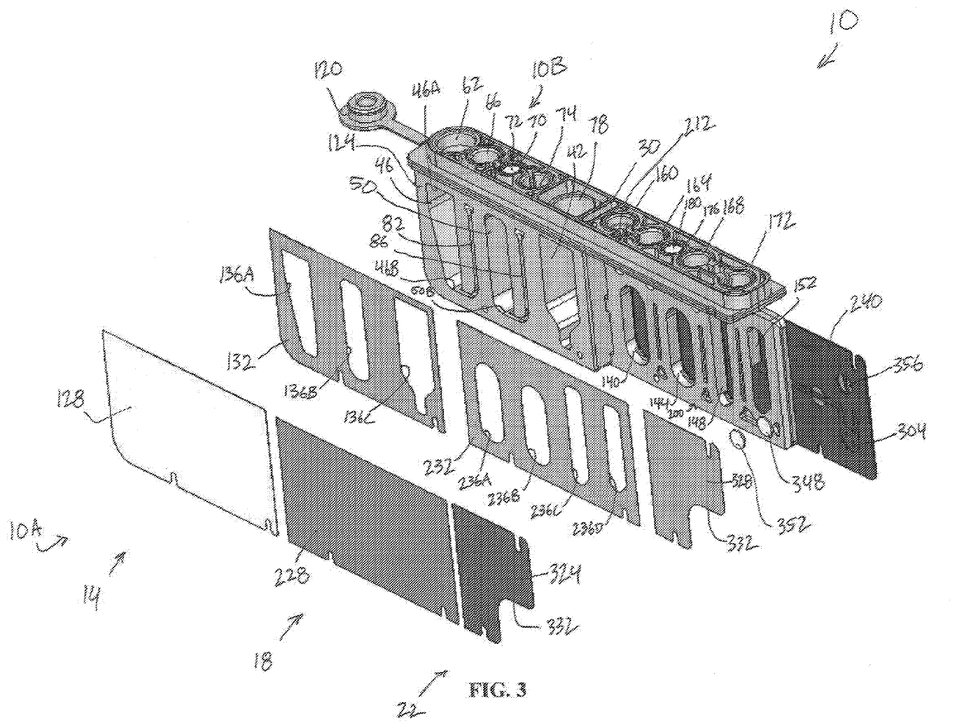

With reference to FIGS. 2 and 3, the portion of the connector 30 (or docking section) coupled to the storage section 14 includes an inlet 62 (e.g., a specimen inlet), a first access port 66 fluidly connected to the first chamber 46, a first vent 70 fluidly connected to the second chamber 50, a second access port 74 fluidly connected to the second chamber 50, and an opening 78 to the cavity 42. In the illustrated embodiment, a filter 72 is positioned within the first vent 70. A first channel 82 fluidly connects the first chamber 46 with the first access port 66. Likewise, a second channel 86 fluidly connects the second chamber 50 with the second access port 74. As explained in greater detail herein, the first access port 66 and the second access port 74 are configured to interface with the transfer capsule 26.

In some embodiments, additional chambers in the storage section (e.g., third storage chamber, fourth storage chamber, etc.) may each comprise one or more of liquid inlets, access ports (e.g., that interface with the transfer capsule), vents, etc. (e.g., within the storage section and/or the associated connector).

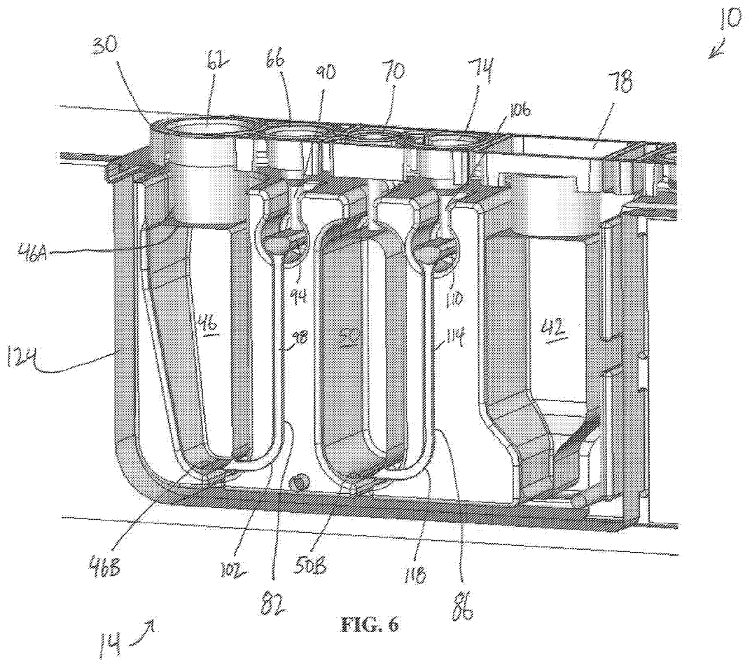

With reference to FIG. 6, the first channel 82 includes a first portion 90 coupled to the first access port 66, a second portion 94 in fluid communication with the first portion 90 and extending approximately perpendicular to the first portion 90. The first channel 82 further includes a third portion 98 in fluid communication with the second portion 94 and extending approximately perpendicular to the second portion 94 and approximately parallel to the first portion 90. Also, the first channel 82 includes an arcuate portion 102 positioned between the third portion 98 and the first chamber 46. The first chamber 46 has a first end 46A and a second end 46B, with the first end 46A positioned closer to the connector 30 (or docking section) than the second end 46B. In the illustrated embodiment, the arcuate portion 102 is coupled to the second end 46B of the first chamber 46. In other words, the first channel 82 fluidly connects the first access port 66 to the end 46B of the first chamber 46 positioned opposite from the inlet 62.

With continued reference to FIG. 6, the second channel 86 is similar to the first channel 82 and likewise includes a first portion 106, a second portion 110, a third portion 114, and an arcuate portion 118 coupled to an end 50B of the second chamber 50 positioned opposite the connector 30 (or docking section).

In some embodiments, the connector 30 (or docking section) includes a cap 120 that is corresponding received within the inlet 62 once a specimen has been positioned within the first chamber 46. In the illustrated embodiment, the cap 120 is moveable between an open position (FIG. 2) in which the cap 120 is removed from the inlet 62 and a closed position in which the cap 120 is positioned within the inlet 62. In some embodiments, a filter, similar to filter 72, is positioned in the cap 120 to permit venting of the first chamber 46 during fluid aspiration/dispensing via the first access port 66.

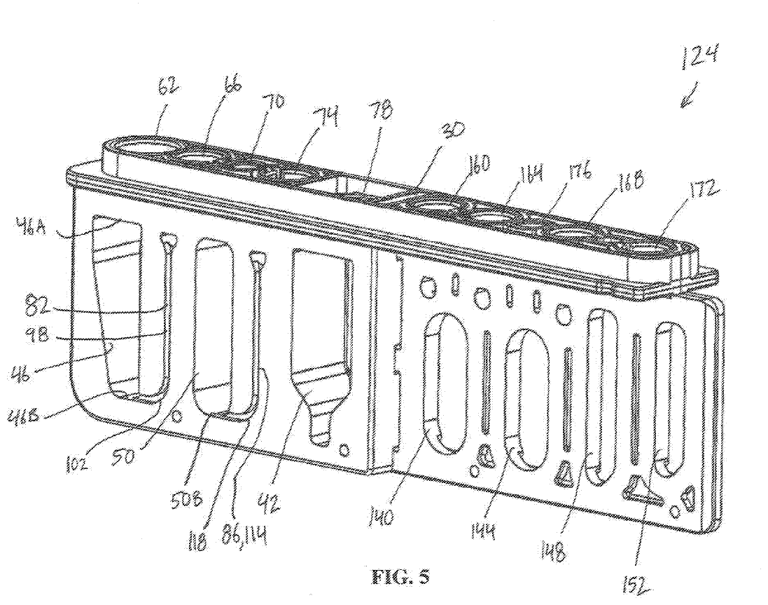

With reference to FIG. 3, the cartridge 10 includes a main body 124 (FIG. 5) in which the first chamber 46, the second chamber 50, and the cavity 42 are at least partially formed in. In some embodiments, the body 124 is an integrally molded as a unitary component.

In some embodiments, the cartridge 10 further includes a first cover 128 that is coupled to the body 124, for example, with a first adhesive layer 132. In the illustrated embodiment, the first cover 128 is a film cover and the first adhesive layer 132 is a pressure sensitive adhesive (PSA). In the illustrated embodiment, the first cover 128 and the first adhesive layer 132 correspond to the storage section 14 of the cartridge 10. The first adhesive layer 132 includes cutouts 136A, 136B, 136C corresponding to the first chamber 46, the second chamber 50, and the cavity 42, respectively. In other embodiments, the cartridge 10 further includes a first cover 128 that is coupled to the body 124, for example, by heat sealing.

In the illustrated embodiment, the first cover 128 is a heat transfer surface for the first chamber 46 and the second chamber 50. In other words, the first cover 128 is an advantageously thin film to improve heat transfer from outside the cartridge 10 to the first and second chambers 46, 50. Opposite the first cover 128, the first chamber 46, the second chamber 50, and the cavity 42 are defined by the rigid body 124 (FIG. 4). In other words, the first cover 128 and the body 124 at least partially define the first chamber 46, the second chamber 50, and the cavity 42 in the illustrated embodiment.

In some embodiments, the second chamber 50 is configured to contain a volume of buffer. In some embodiments, the device is configured such that the transfer capsule 26 can withdraw the buffer from the second chamber 50 via the second channel 86 and move the buffer to one or more other chambers within the storage and/or processing sections. In some embodiments, the first chamber 46 is configured to receive a sample or specimen. A sample or specimen may be a liquid (e.g., body fluid (e.g., blood, saliva), buffer-dissolved sample, etc.) or a solid (e.g., a swab tip). In some embodiments, the device is configured such that the transfer capsule 26 can withdraw the sample (e.g., combined with buffer) from the first chamber 46 via the first channel 82, and move the sample to one or more other chambers within the storage and/or processing sections.

With reference to FIGS. 2 and 3, the processing section 18 includes a third chamber 140, a fourth chamber 144, a fifth chamber 148, and a sixth chamber 152. In other embodiments, the processing section 18 includes more than four chambers or fewer than four chambers. For example, in some embodiments, the processing section 18 includes fewer than four chambers. In other embodiments the processing section 18 includes at least five chambers. In some embodiments, a processing section may contain fewer (e.g., 1, 2, or 3) chambers or more chambers (e.g., 5, 6, 7, 8, or more) than depicted in the figures herein. The chambers may be referred to by a suitable numerical depending on the number of chambers in the storage and processing sections. In the illustrated embodiment, the processing section 18 has a width 156 that is within a range of approximately 1 mm to approximately 3 mm (e.g., 1 mm, 1.25 mm, 1.5 mm, 1.75 mm, 2.0 mm, 2.25 mm, 2.5 mm, 2.75 mm, 3.0 mm, and ranges therebetween), wider and narrower processing sections are within the scope herein. As explained in greater detail herein, in the third, fourth, fifth and sixth chambers 140, 144, 148, 152 targets are extracted, purified and/or bound to magnetic beads.

With continued reference to FIG. 3, the portion of the connector 30 (or docking section) coupled to the processing section 18 includes a third access port 160 fluidly connected to the third chamber 140, a fourth access port 164 fluidly connected to the fourth chamber 144, a fifth access port 168 fluidly connected to the fifth chamber 148, and a sixth access port 172 fluidly connected to the sixth chamber 156. The connector 30 (or docking section) further includes a second vent 176 that fluidly connects each of the third, fourth, fifth and sixth chambers 140, 144, 148, 152 to atmosphere. In the illustrated embodiment, a filter 180 is positioned within the second vent 176. In some embodiments, any vents herein may comprise a filter to prevent debris from entering the associated chamber and/or to prevent liquid escape from the chamber (e.g., as an aspirate).

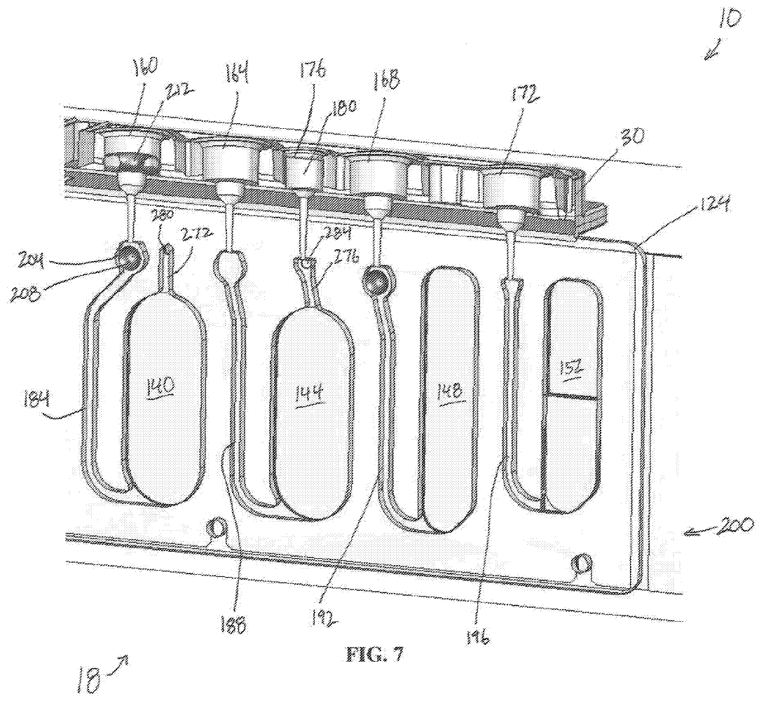

With reference to FIG. 7, a third channel 184 fluidly connects the third chamber 140 with the third access port 160, and a fourth channel 188 fluidly connects the fourth chamber 144 with the fourth access port 164. Likewise, a fifth channel 192 fluidly connects the fifth chamber 148 with the fifth access port 168, and a sixth channel 196 fluidly connects the sixth chamber 152 with the sixth access port 172. In the illustrated embodiment, each of the channels 184, 188, 192, 196 fluidly connect to the corresponding chamber 140, 144, 148, 152 at an end 200 positioned opposite from the connector 30 (or docking section).

With reference to FIGS. 7 and 8, the third channel 184 includes a cross channel portion 204 positioned proximate the third access port 160. In some embodiments, a lyophilized reagent 208 (in the shape of a sphere, for example) is positioned in the cross channel portion 204 and is rehydrated when liquid flows through the third channel 184. In other embodiments, a lyophilized reagent is added within any of the channels 184, 188, 192, 196. In some embodiments, one or more of channels 184, 188, 192, 196 contain pockets or other openings to contain reagents (solid or liquid). In some embodiments, one or more of channels 184, 188, 192, 196 do not contain pockets or other openings to contain reagents (solid or liquid). Storing the lyospheres 208 within the channel 184 leading to the chamber 140 helps physically contain the lyospheres 208, and also with improves the resolubilizing the dried reagents effectively and uniformly (i.e., storing the freeze dried lyospheres in a chamber can sometimes cause them to float up with the fluid and not mix as effectively).

With continued reference to FIG. 8, the transfer capsule 26 is illustrated fluidly coupled to the third access port 160. In the illustrated embodiment, a seal 212 (e.g., an O-ring) is positioned within the third access port 160 and improves the sealing between the transfer capsule 26 and the body 124. In some embodiments, any of the access ports in the device may comprise a seal (e.g., O-ring) or may be without a seal. The transfer capsule 26 includes a tip 216 that configurable to extend through the third access port 160 and be at least partially received within the third channel 184 (FIG. 8). The transfer capsule 26 further includes a filter 220 and a transfer chamber 222 positioned between the filter 220 and the tip 216. In the illustrated embodiment, the transfer capsule 26 includes an open end 224 positioned opposite the tip 216. As explained in greater detail herein, fluid may be aspirated from a chamber (e.g., the third chamber 140) into the transfer chamber 222 and transferred to a different location in the cartridge 10.

The transfer capsule 26 provides random access to liquid in the cartridge 10 and also provides the ability to mix solids and fluids. Suspension of solid-phase particles and liquids flowing through a small diameter aperture 218 in the tip 216 of the transfer capsule 26 are subjected to high shear forces, which can break up aggregated particles and aid in desorbing interferents absorbed on their surface. Another advantage of the transfer capsule 26 is that multiple aliquots can be taken out of the common wash buffer without cross contamination because the transfer capsule 26 never comes into contact with the bulk solution.

In some embodiments, the transfer capsule 26 resides within the storage section 14 and/or the processing section 18 of the cartridge device 10. In some embodiments, the transfer capsule resembles a pipette tip. In some embodiments, the tip 216 of the transfer capsule 26 is configured to interface with the access ports connected to the various chambers of the device 10. The open end 224 of the transfer capsule 26 is accessible from the exterior of the device 10. In some embodiments, a pressure source from the exterior of the device (e.g., a component of the instrument that the device is inserted into) interfaces with the open end 224 of the transfer capsule 26. The exterior pressure source applies a negative pressure differential (e.g., decreased pressure with respect to that of the) through the transfer capsule 26 in order to withdraw fluid from the chamber through the access port. The exterior pressure source applies a positive pressure differential (e.g., increased pressure with respect to that of the) through the transfer capsule 26 in order to expel fluid from the transfer capsule 26 through the access port and into the chamber. In some embodiments, a device comprises a single transfer capsule for moving liquids (e.g., sample, buffer, etc.) between chambers. In some embodiments, a device comprises multiple (e.g., 2, 3, 4, etc.) transfer capsules. In some embodiments, the external pressure source interfaces with the transfer capsule, lifts the transfer capsule 26 from the device, moves the transfer capsule 26 to desired location, and then interfaces the transfer capsule 26 with the desired access port. The transfer capsule 26 is contained within the cartridge device. The external pressure source and other components of the instrument, within which the device is inserted, do not contact the liquid reagents within the device. In some embodiments, the transfer capsule is used to transport multiple different liquids between multiple different chambers. In some embodiments, the external pressure source and transfer capsule may disengage and reengage multiple times during an analysis. The use of the transfer capsule and external pressure source allows for inter-chamber liquid transfer within the storage and processing sections, as well as mixing liquids in the chambers, without valves and without regard for the relative location of the chambers in the device (e.g., ‘skipping’ a chamber).

With reference to FIG. 3, the main body 124 (FIG. 5) at least partially forms the third chamber 140, the fourth chamber 144, the fifth chamber 148, and the sixth chamber 152. On a first side 10A of the cartridge 10 further includes a cover 228 that is coupled to the body 124 with an adhesive layer 232. In the illustrated embodiment, the cover 228 is a film cover and the adhesive layer 232 is a pressure sensitive adhesive (PSA). In some embodiments, the cover 228 is thin to allow rapid heat transfer, but is capable of withstanding temperatures up to 100° C. In some embodiments, the cover 228 is chemically compatible (e.g., non-reactive, chemically inert, etc.). In the illustrated embodiment, the cover 228 and the adhesive layer 232 correspond to the processing section 18 of the cartridge 10. The adhesive layer 232 includes cutouts 236A, 236B, 236C, 236D corresponding to the third chamber 140, the fourth chamber 144, the fifth chamber 148, and the sixth chamber 152, respectively.

With reference to FIG. 4, on a second side 10B of the cartridge 10 (opposite the first side 10A) the processing section 18 includes a first laminated layer 240, a second laminated layer 244, a cover 248, and three layers of adhesive 252, 256, 260 positioned therebetween. In the illustrated embodiment, the first layer adhesive 252 couples the first laminated layer 240 to the main body 124. Likewise, the second layer of adhesive 256 couples the second laminated layer 244 to the first laminated layer 240. Finally, the third layer adhesive 260 couples the cover 248 to the second laminated layer 244. In other embodiments, the processing section 18 includes any number of lamination layers and layers of adhesive coupled therebetween. Films 240 and 248 are specifically chosen for their optical clarity, fluorescence compatibility, fast and efficient heat transfer, chemical compatibility, and ability to withstand high temperatures (approximately 100° C.).

With continued reference to FIG. 4, each of the first laminated layer 240, the second laminated layer 244, the adhesive layers 252, 256, and 260 each include cutouts 264A, 264B, 264C, 264D corresponding to the chambers 140, 144, 148, 152. In the illustrated embodiment, the second laminated layer 244 and the adhesive layer 252 each at least partially define a common vent channel 268. Each of the third, fourth, fifth, and sixth chambers 140, 144, 148, 152 are fluidly connected to the second vent 176 by the common vent channel 268. Specifically, the common vent channel 268 intersects with the fifth and sixth chambers 148, 152. The third and fourth chambers 140, 144 are fluidly connected to the common vent channel 268 by connecting channels 272, 276 and apertures 280, 284 formed in the body 124 (FIG. 7). In other words, the connecting channel 272 and the aperture 280 are positioned between the third chamber 140 and the common vent channel 268. Likewise, the connecting channel 276 and the aperture 284 are positioned between the fourth chamber 144 and the common vent channel 268. In the illustrated embodiment, the common vent channel 268 is at least partially bounded by the first laminated layer 240 and the adhesive layer 260.

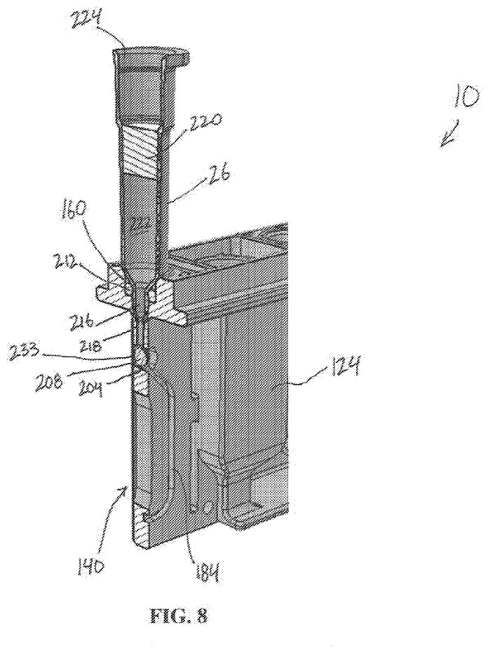

With reference to FIG. 9, the microfluidic section 22 includes a reaction chamber 304, a microfluidic vent channel 308, and a microfluidic inlet channel 312. The microfluidic vent channel 308 is fluidly connected to the reaction chamber 304. The microfluidic inlet channel 312 is fluidly connected to the reaction chamber 304 and the sixth chamber 152 in the processing section 18. In other words, the microfluidic inlet channel 312 is fluidly connects the sixth chamber 152 in the processing section 18 to the microfluidic section 22. In the illustrated embodiment, the microfluidic inlet channel 312 connects to a side portion 316 of the sixth chamber 152 (FIG. 4), where the side portion 316 is positioned between where the sixth channel 196 connects to the chamber 152 and the sixth access port 172. In other words, the microfluidic inlet channel 312 connects to the side portion 316 in the middle of the sixth chamber 152. The microfluidic inlet channel 312 acts as an interface between the processing section 18 and the microfluidic section 22 and facilitates easy transfer of liquids and PMPs to the microfluidic section 22, which has openings that are not directly accessible by a pipettor (transfer capsule). The offset of chamber 196 in comparison to chamber 192 in the vertical orientation also helps achieve high hydrostatic head, which also facilitates easy transfer of liquids to section 22.

With continued reference to FIG. 9, the reaction chamber 304 is positioned between the microfluidic inlet channel 312 and the microfluidic vent channel 308. In some embodiments, a lyophilized master mix 320 is positioned within the reaction chamber 304, where PCR, for example, is performed therein.

With reference to FIGS. 3 and 4, the microfluidic section 22 of the illustrated embodiment is at least partially defined by a cover 324 and an adhesive layer 328 (FIG. 3). In addition, the microfluidic section 22 is at least partially defined by the first laminated layer 240, the adhesive layer 256, the second laminated layer 244, the adhesive layer 260, and the cover 248. As such, in the illustrated embodiment, the microfluids section 22 includes at least seven layers (including adhesive layers). The microfluid section 22 defines a width 334. In the illustrated embodiment, the width 334 is smaller than the width 156, which is smaller than the width 54. The cover 324 and the adhesive layer 328 include a cutout 332 corresponding to the reaction chamber 304. In other words, the cutout 332 in the cover 324 and the adhesive layer 328 expose the reaction chamber 304 for improved optical detection and heater interface.

With reference to FIGS. 4 and 9, the microfluidic vent channel 308 includes a first portion 336 formed in the adhesive layer 260, a second portion 340 formed in the adhesive layer 256, and a third portion 344 formed in the adhesive layer 260. As such, the second portion 340 is offset from (i.e., positioned within a different plane) the first and third portions 336, 344 of the microfluidic vent channel 308. The third portion 344 of the microfluidic vent 308 is fluidly connected to the common vent channel 268.

With reference to FIG. 4, the adhesive layers in the cartridge 10 (e.g., adhesive layer 256, 260, 328) add functionality to the processing and microfluid sections 18, 22. Adhesive is conventionally used to bond films together to create a laminated structure. However, the adhesive layers in the cartridge 10 provide additional functions. First, the adhesive layers can be exposed to create a location to which lyophilized reagents may be bonded. For example, in some embodiments, the PCR master mix 320 in the reaction chamber 304 is adhered to an adhesive layer exposed to the reaction chamber 304. In other embodiments, the spheres of lyospheres (e.g., the reagent 208 in the third channel 184) are secured to an exposed portion 233 (FIG. 8) of the adhesive layer 252. In some embodiments, the reagent 208 is adhesively bonded to PSA 252 on the opposite side from where they are inserted.

The second additional function provided by the adhesive layers is that channels are formed in the adhesive layers to equilibrate air pressure within the chambers. In the illustrated embodiment, the chambers 140, 144, 148, 152 in the processing section 18 are fluidly connected to the common vent channel 268, which is at least partially formed in the adhesive layer 256 (FIG. 4). In other embodiments, the microfluidic inlet channel 312 and the microfluidic vent channel 308 are at least partially formed in the adhesive layers 256, 260. Utilizing the adhesive layers to partially form channels reduces the overall size the of the cartridge 10.

With continued reference to FIG. 9, the microfluid section 22 includes a first wax seal 348 and a second wax seal 352. The first wax seal 348 is positioned adjacent the microfluidic vent channel 308 and the second wax seal 352 is posited adjacent the microfluidic inlet channel 312. To maintain stable concentrations of reagents and amplicon, PCR is conducted in a closed system (e.g., the reaction chamber 304 is sealed). The wax seals 348, 352 are configured to seal the reaction chamber 304 from two ends (i.e., the inlet end and the vent end). The open-air microfluidic vent channel 308 leading from the reaction chamber 304 is utilized for initial buffer fluid priming of the dried reagents (via channel 312) and air purge. After priming, paramagnetic particles carrying the target are transferred into the reaction chamber 304 through the microfluidic inlet channel 312. Once the target is in the reaction chamber 304, the microfluidic vent channel 308 is closed (i.e., sealed) by melting the first wax seal 348 (by, for example, a heater). The molten wax fills the void space and nearby microfluid vent channel 308 and then hardens, sealing the microfluid vent channel 308. After the first wax seal 348 is melted, the second wax seal 352 is then melted in a similar fashion, sealing off the microfluid inlet channel 312 and closing the reaction chamber 304. Therefore, in some embodiments, the first wax seal 348 in the microfluidic vent channel 308 is melted and hardened before the second wax seal 352 in the microfluidic inlet channel 312. Doing so ensures any air entrapped around the second wax seal 352 builds pressure to prevent molten wax from the second wax seal 352 from flowing into the reaction chamber 304. Any entrapped air prefers to flow away from reaction chamber 304 via channel 312 towards the sixth chamber 152. Wax in the reaction chamber 304 would affect the fluorescent optical readings.

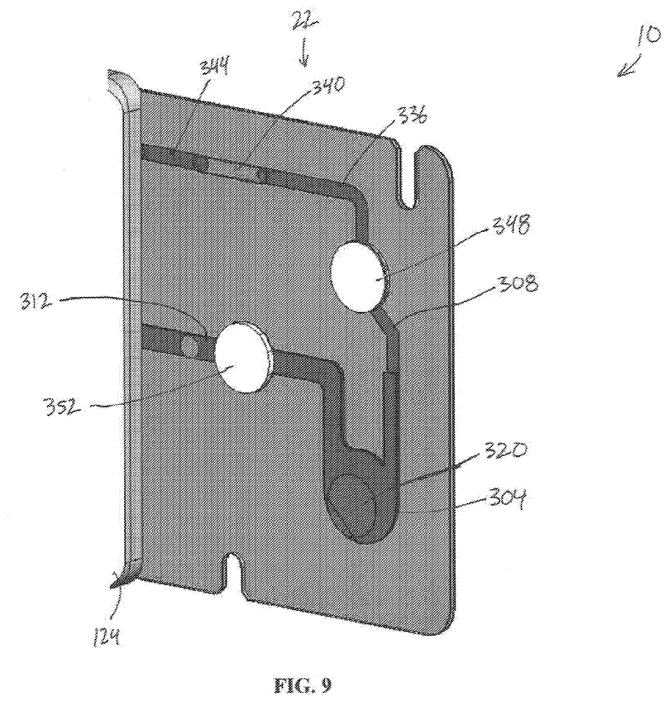

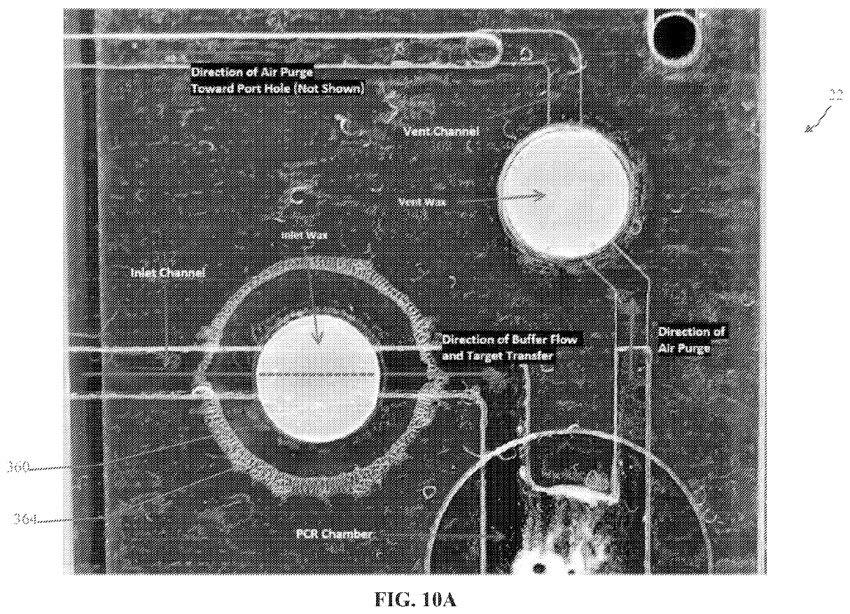

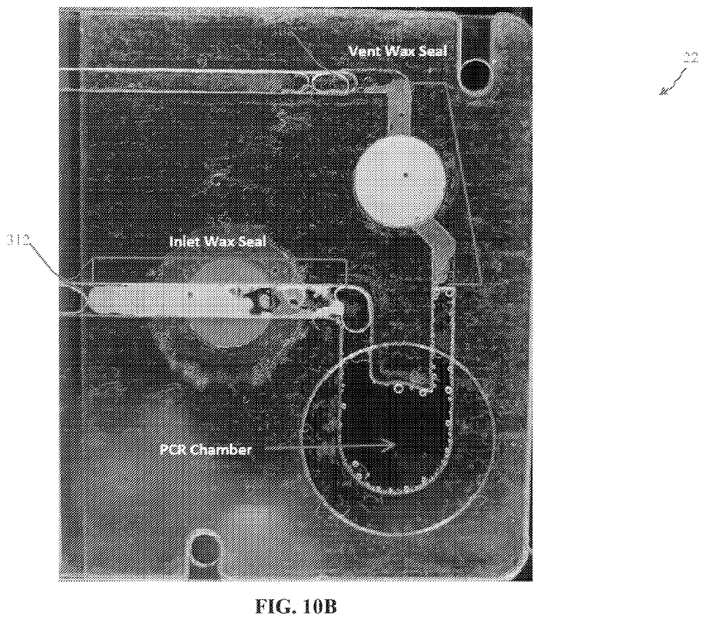

With reference to FIGS. 10A and 10B, the first and second wax seals 348, 352 are initially in a first solid state with the corresponding channels 308, 312 open (FIG. 10A) and are modifiable to a second solid state with the corresponding channels 308, 312 sealed (i.e., closed) (FIG. 10B). The wax seals 348, 352 enter a molten state between the first solid state and the second solid state. In the illustrated embodiment, the first and second wax seals 348, 352 are cylindrically shaped in the initial, first solid state (FIG. 10A) and do not obstruct or seal their corresponding channels 308, 312. In response to the application of heat, the first and second wax seals 348, 352 melt and flow into the channels 308, 312. After the application of heat is removed, the molten wax rehardens and enters the second solid state (FIG. 10B) and forms a solid wax seal positioned within the channels 308, 312.

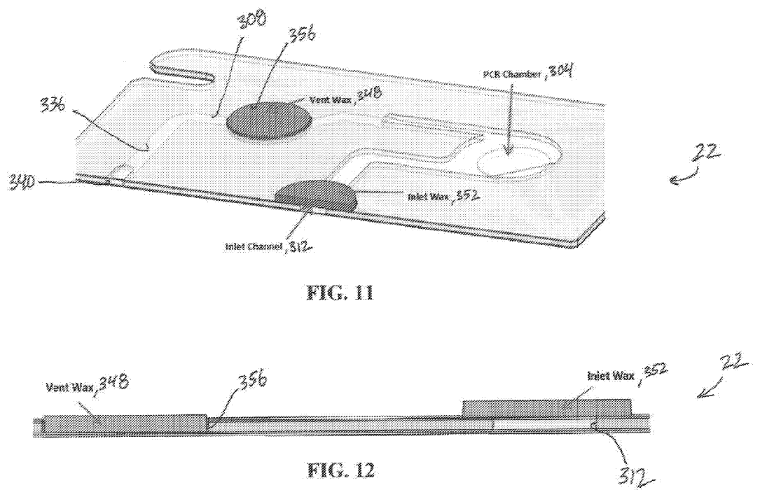

With reference to FIGS. 11 and 12, the first wax seal 348 in the first solid state is initially positioned above the microfluidic vent channel 308 within a cutout 356 formed in the laminated layers 240, 244 and the adhesive layer 256 (FIG. 4). The first wax seal 348 initially rests on top of a layer of adhesive (260) to improve bond quality at the interface. In the initial solid state of the first wax seal 348, air can easily pass around and beneath the wax seal 348. When the wax seal 348 is melted, molten wax fills the empty surrounding space. In some embodiments, the volume of first wax seal 348 is less than the volume of the second wax seal 352.

With continued reference to FIGS. 11 and 12, the second wax seal 352 positioned adjacent the microfluidic inlet channel 312 extends beyond the laminate layers, creating a tented air pocket 360 (FIG. 10A) around the perimeter of the second wax seal 352. In other words, the second wax seal 352 causes the cover 324 to deform during assembly resulting in the tented air pocket 360. Despite the second wax seal 352 being positioned above the microfluidic inlet channel 312, fluid and target transfer through the microfluid inlet channel 312 remains possible by utilizing hydrostatic head at the onset of the microfluidic inlet channel (contained within chamber 152) and the amount of detergent in the fluid.

In some embodiments, the microfluidic inlet channel 312 is taller than the microfluidic vent channel 308. The microfluidic vent channel 312 has a cross-sectional area of approximately 0.051 mm² (e.g., 0.051 mm tall×1 mm wide). Likewise, the microfluidic inlet channel 312 has a cross-sectional area of approximately 0.54 mm² (e.g., 0.36 mm tall×1.5 mm wide). The microfluidic vent channel 308 has a smaller cross-sectional area than the microfluid inlet channel 312 because the microfluid vent channel 308 directs only airflow whereas the microfluid inlet channel 312 must allow passage of liquid buffer and solid particles containing genetic targets. Therefore, the amount of wax required in the wax seal 352 for the microfluid inlet channel 312 is greater than the amount of wax in the wax seal 348 for the microfluid vent channel 308.

In some embodiments, the tented air pocket 360 is approximately 0.38 mm taller than the surrounding laminate. When the wax seals 352 is melted by clamping heaters, the tented air pocket 360 is depressed. Air remaining entrapped within hardened wax seals creates potential for fluid leakage. As such, it is important the air has a route to exit the system during the wax melting process to not compromise seal integrity.

In the illustrate embodiment, spacing of at least approximately 2 mm is provided between any laser cut feature and an edge of the laminate to provide sufficient surface area for a strong adhesive bond to form. In other words, narrow adhesive contact areas are vulnerable to leakage of leakage and failure. Specifically, the perimeter 364 of the tented air pocket 360 is positioned at least approximately 2 mm away from the reaction chamber 304. In the illustrated embodiment, there is at least approximately 2 mm of spacing from the tented air pocket and any exposed laminate edge.

The wax seals 348 and 352 provide several advantages. The hardened wax seals 348, 352 are advantageously configured to withstand the pressures in the reaction chamber 304 experienced during thermal cycling, which involves alternated clamping of the reaction chamber with heater temperatures in the range of approximately 50° C. and approximately 95° C. In other words, the combination of high temperatures and fluid displacement from mechanical clamping puts stress on the wax seals 348, 352 that are withstood. Although the heaters may not directly contact wax seals during thermal cycling, wax with a high melting temperature (e.g., paraffin wax with a melting temperature of at least approximately 85° C.) is selected in some embodiments to ensure the wax seals are not inadvertently re-melted by heaters associated with the reaction chamber 304.

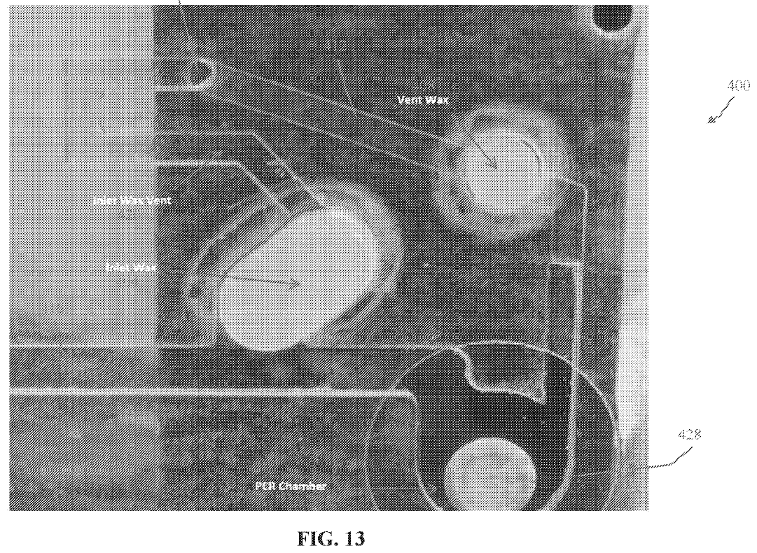

In the illustrated embodiment, the wax seals are initially cylindrically shaped (i.e., coin-shaped) (e.g., approximately 4.5 mm in diameter by approximately 0.43 mm thick). The rotational symmetry of a circular geometer of a wax seal reduces the risk of misplacement during manufacturing of the cartridge 10. Furthermore, a design with identical wax seals simplifies production. In other embodiments, the wax seals were initially elliptical-shaped (FIG. 13).

The wax seals 348, 352 can be melted within a range of approximately 86° C. (i.e., the wax melting point) and approximately 95° C. (i.e., the default temperature setting of a PCR heater). The melting duration, or that the PCR heaters are clamped onto a wax seal, can be modulated in tandem with the melting temperature to ensure a good seal. For example, if a wax seal is melted at too high of a temperature for too long, molten wax will diffuse further away from the sealing site—reducing the material density and mechanical integrity of the seal. In some embodiments, the melting procedure melts the wax seals 348, 352 by applying a hotter heater for a duration within a range of approximately 4 to approximately 5 seconds. Then, the wax seals 348, 352 are clamped with a cooler heater with a setpoint below the wax melting temperature for a duration of approximately 1.5 second. To reduce overall processing time, the wax seals 348, 352 are melted while the hot PCR heaters are cooling from approximately 95° C. to approximately 86° C. (rather than at a fixed temperature).

In some embodiments, the density of the wax seals 348, 352 is approximately 0.9 g/mL, which is slightly less dense than the fluids surrounding them at 1 g/mL. In the illustrated embodiment, gravity acts downwards during the melting and subsequent hardening of the wax seals. As such, the orientation of gravity can influence the movement of the molten wax. For example, molten wax may flow upwards when gravity is acting downwards. The direction of the molten wax flow is also affected by the surface area and position of the heater utilized to melt the wax seals. For example, when clamping with a heater, if it is not concentric but rather offset in one direction, the molten wax will tend to flow in the offset direction. Likewise, the heater clamping force is modulated as a function of the relative positions of the front and back sides of the heater, which can be mounted on low spring-constant springs, for example. In some embodiments, the plastic laminate layers are less rigid and capable of deforming in response to the heater clamping force, extruding and pushing the wax seals beyond the boundary of the heater surface area.

In some embodiments, the molten wax seals are cooled by clamping the molten wax seals with a cooler heater (i.e., a heater with a temperature less than the wax melting temperature). In other embodiments, the molten wax seals are cooled hardening in ambient air. Clamping the molten wax seals with the cooler heaters to cool the wax cause the wax to harden more quickly. Time to cool in ambient air is approximately 6 to approximately 8 second, whereas time to cool by clamping is approximately 2 seconds.

In some embodiments, the bonding of the wax seals 348, 352 is improved by exposing the paraffin wax to a layer of acrylic-based adhesive tape, instead of other plastic films such as polyester or polycarbonate. Improved bond quality between melted wax and a channel wall can decrease the likelihood of fluid leakage through the hardened seal.

Overall, the cartridge 10 provides several advantages. The cartridge 10 is self-contained with on-board liquid and freeze-dried reagents that does not require refrigeration. The cartridge 10 allows processing of a variety of input sample types, including from transfer pipettes, nasal swabs, and the like. Inter-chamber liquid transfer with the cartridge 10 achieves random access and rapid mixing without any valves. As used herein, random access means a liquid on one chamber can be transferred to any other chamber without regard for its location in the cartridge 10. Because the cartridge 10 achieves random access of liquids the cartridge 10 is adaptable to different processing protocols. In addition, the random access in the cartridge 10 enables the washing of paramagnetic particles (PMPs) multiple times by disposing of dirty wash in empty processing chambers. For example, dirty wash in the fifth chamber 148 is disposed of in the third chamber 140 where lysis was done. In some embodiments, PMPs are used to mix the fluid inside microfluid section 22 via a magnetic coupling. In addition, the random access enables the cartridge 10 to store PMPs in the fourth chamber 144 and add the PMPs to the third chamber 140 after probes have had time to bind to targets at an elevated temperature—eliminating the need for additional heaters.

Another advantage of the cartridge 10 is that the wet storage section 14 and the dry reagents in the processing and microfluid sections 18, 22 are separated by the cavity 42 that receives the transfer capsule 26. In other words, the cavity 42 increases the distance between the dry reagents and the liquid buffer in the cartridge 10 to improve shelf life. Two separate heat-sealed foil lids 34, 38 also provide further separation between the storage section 14 and the processing section 18.

An additional advantage of the cartridge 10 is that liquids can be flowed into chambers at high velocities (i.e., laboratory bench pipette mixing) which provides good mixing of reactants. Speed, volume, and time delays allow for flexibility. The cross-sectional geometry of the channels (e.g., the third channel 184) facilitate fluid mixing. Furthermore, air can be injected in the chambers creating bubbles that pass from the bottom of the chamber to the vent in order to mix fluids via chaotic eddies and vortices. In some embodiments, air can be vented through channels to a single common filter (e.g., the filter 180 in the vent 176).

Another advantage of the cartridge 10 is that is minimizes the chance of specimen or processed specimen leaking out of the cartridge. When the cartridge 10 is upright (i.e., as viewed in FIG. 1), liquid is far from the outlet channel and hydrostatic pressure pulls fluid away from the outlet. When the cartridge 10 is placed upside down, the liquid is far from the inlet to the channels and hydrostatic pressure is pulling liquid away from the inlet. When the cartridge 10 is placed on its side, gravity is across the chamber so there is no hydrostatic head to cause liquid to flow towards the outlet. The dimensions and shape of the channels work against any capillary driven flow.

With reference to FIG. 13, a microfluidic section 400 (similar to the microfluidic section 22) according to another embodiment is illustrated. The microfluidic section 400 includes an elliptical inlet wax seal 404 and a circular vent wax seal 408. In some embodiments, the elliptical inlet wax seal 404 has a volume of at least approximately 2 times the volume of the vent wax seal 404. The vent wax seal 404 is positioned directly over a vent channel 412. The inlet wax seal 404 is positioned offset from (as viewed in FIG. 13), but in the same plane as, a microfluidic inlet channel 416. In the illustrated embodiment, the inlet wax seal 404 has a corresponding wax vent channel 420 that merges with a venting port 424 positioned downstream of a reaction chamber 248. The wax vent channel 420 permits air to escape through the venting port 424 and away from the microfluidic inlet channel 416 during the melting and hardening processes.

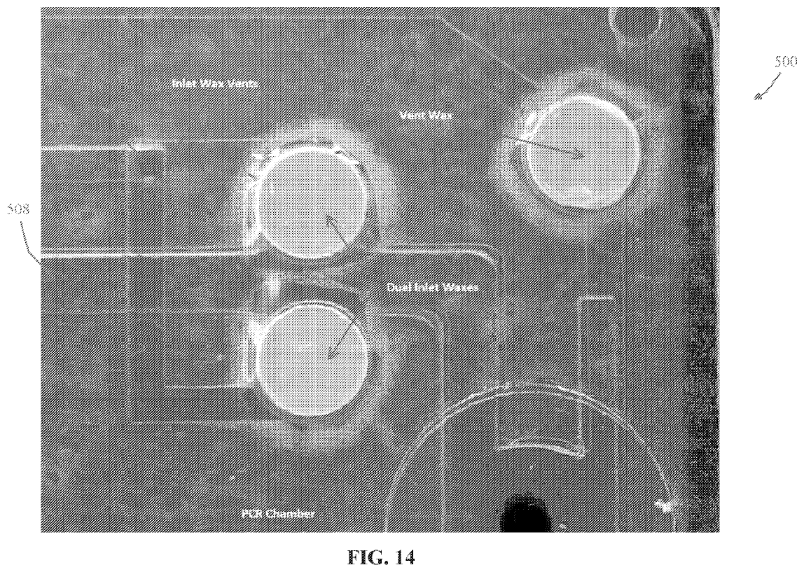

With reference to FIG. 14, a microfluids section 500 according to another embodiment is illustrated. The microfluids section 500 includes three identical wax seals 504A, 504B, 504C in an initial cylindrical shape. In the illustrated embodiment, each of the wax seals 504A, 504B, 504C in an initial state is approximately 4.0 mm in diameter and approximately 0.51 mm thick. Identical wax seals reduce manufacturing complexity by using uniform dimensions. In the illustrated embodiment, the wax seal 504A and the wax seal 504B are positioned offset from (as view in FIG. 14), but in the same plane as a microfluidic inlet channel 508. In other words, the wax seals 504A, 504B in an initial state are coplanar with the microfluidic inlet channel 508. Each of the inlet wax seals 504A, 504B includes a corresponding wax vent channel 512A, 512B that merge at a common venting port 516.

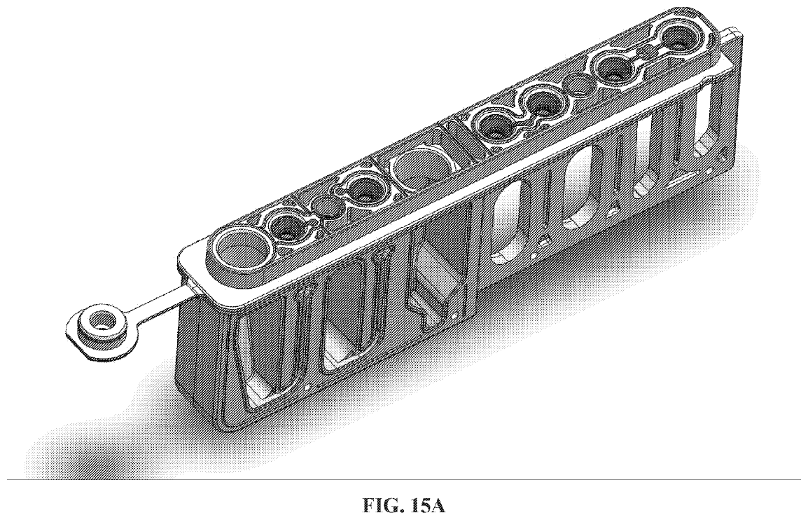

With reference to FIGS. 15A and 15B, each access port is depicted with overmolded seals (blue) in the access ports for the various chambers. In some embodiments, the overmolded seals comprise a thermoplastic elastomer that is molded onto the already formed cartridge in a subsequent molding process. In some embodiments, a solid bond is created between the overmolded seal and the cartridge material. In some embodiments, overmolding eliminates the need to install an O-ring on an access port, which can be more difficult to keep in place during use. Like other access port seals that may find use in embodiments herein (e.g., O-rings), the overmolded seals create an air-tight fluid path between the cartridge and the tip of the transfer capsule. In some embodiments, all or a portion (e.g., 1, 2, 3, 4, 5, 6, 7, or more) of the access ports on the cartridge comprise overmolded seals.

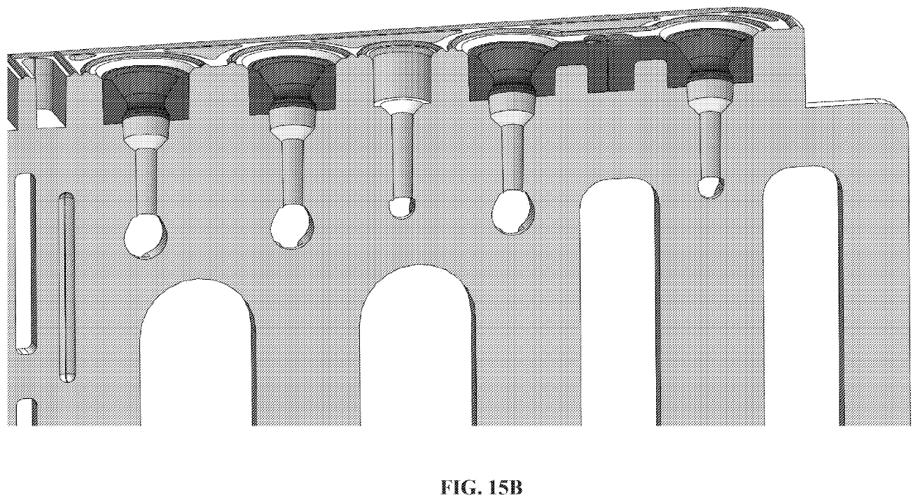

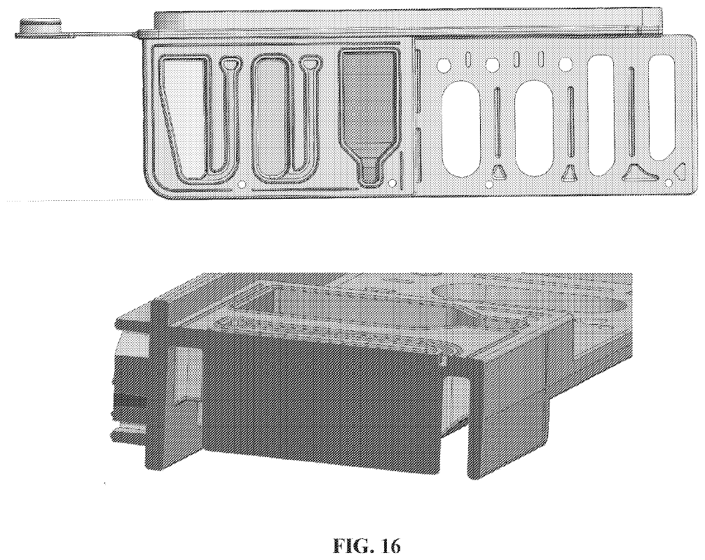

In some embodiments, a film or cover (e.g., the first cover 128, from FIG. 3) is heat sealed onto the cartridge. With reference to FIG. 16, in some embodiments, one surface that is to be heat sealed to another comprises energy directors (e.g., green lines in FIG. 16). The energy directors are raised surfaces where melt can be initiated to ensure that solid and complete seals are formed around key regions (e.g., chambers). In some embodiments, energy directors are linear raised surfaces formed from the bulk plastic of the molded main body. The film is attached with a heated press on the outside of the film. Upon application of the heat, the energy directors are melted and when the melted plastic cools and solidifies, it glues the film to the cartridge body.

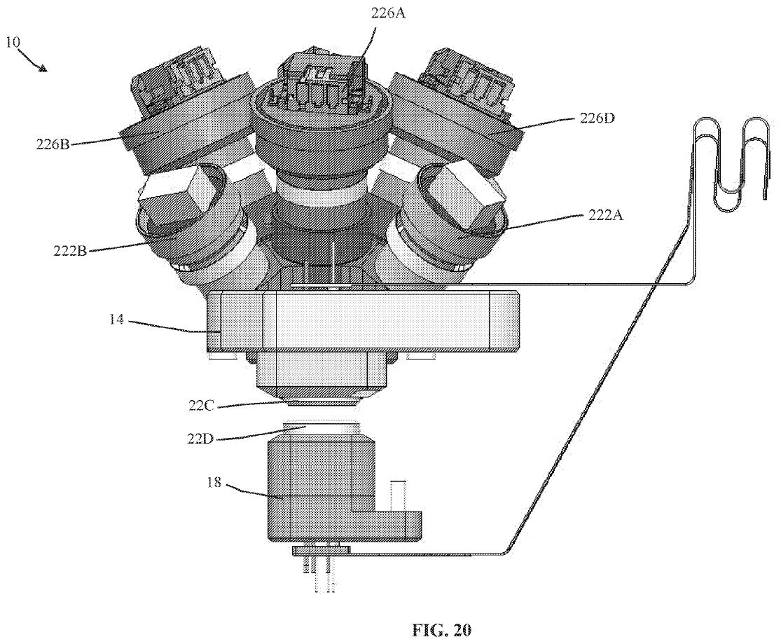

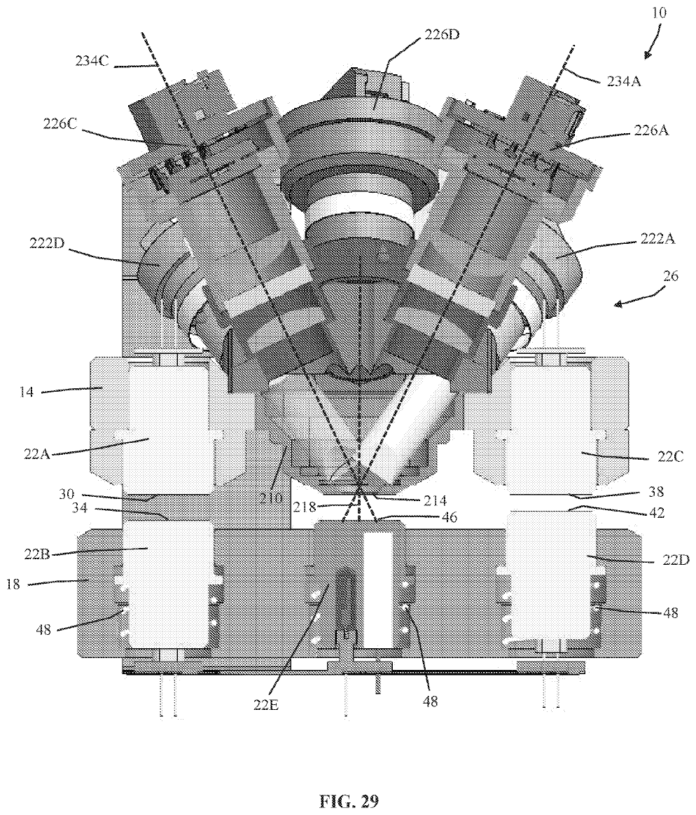

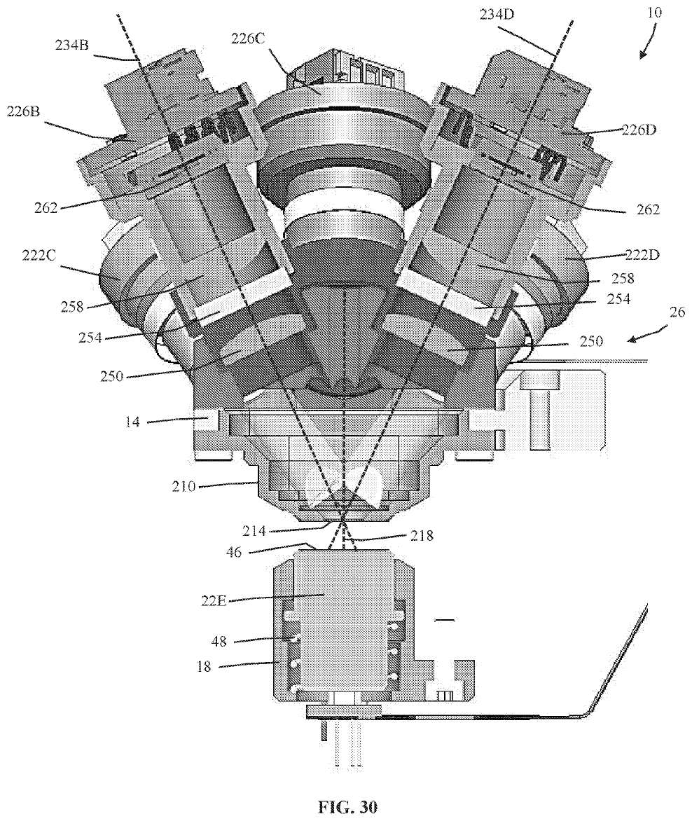

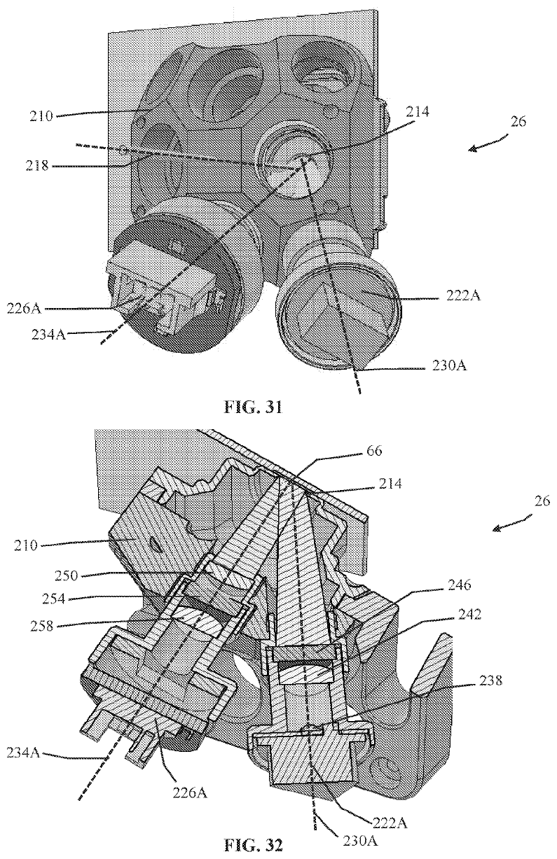

The cartridge devices and components depicted in FIGS. 1-30 is exemplary and depicts a preferred embodiment of the present invention. However, other cartridges comprising different combinations and configurations of the elements depicted in FIGS. 1-30 and/or described herein are within the scope of the invention. Various embodiments and aspects of the present technology are described in patents and publications and understood to those in the field. Exemplary patents and publications include U.S. Pat. Pub. No. 2020/0190506; PCT Pub. No. WO2018226891; and PCT Pub. No. WO2018218053; each of which is incorporated by reference in their entireties.

### III. Instrument

As described throughout, the cartridge devices herein find use with instruments that provide components and functionalities that are absent from the devices. In some embodiments, the cartridge and complementary instrument (e.g., the heating and optics assembly 10) form a system capable of sample processing and analyte detection/quantification. In some embodiments, such systems are provided herein.

In some embodiments, an instrument for use in embodiments herein comprises one or more heaters. In some embodiments, a heater is capable of regulating the temperature of the liquid in a chamber. In some embodiments, heaters reside within the instrument on two sides of a chamber to effectively transfer heat to the chamber. In some embodiments, one or two heaters within the instrument, residing adjacent to a chamber, bring liquid within the chamber to a desired temperature (e.g., 30° C., 35° C., 40° C., 45° C., 50° C., 55° C., 60° C., 65° C., 70° C., 75° C., 80° C., 85° C., 90° C., 95° C., or ranges therebetween) within 30 seconds (e.g., 1 s, 2 s, 3 s, 4 s, 5 s, 6 s, 7 s, 8 s, 9 s, 10 s, 11 s, 12 s, 13 s, 14 s, 15 s, 16 s 17 s 18 s, 19 s, 20 s, 21 s, 22 s, 23 s, 24 s, 25 s, 26 s, 27 s, 28 s, 29 s, 30 s, or ranges therein) depending upon number of heaters, the proximity of the heaters to the chamber, and the volume of the liquid.

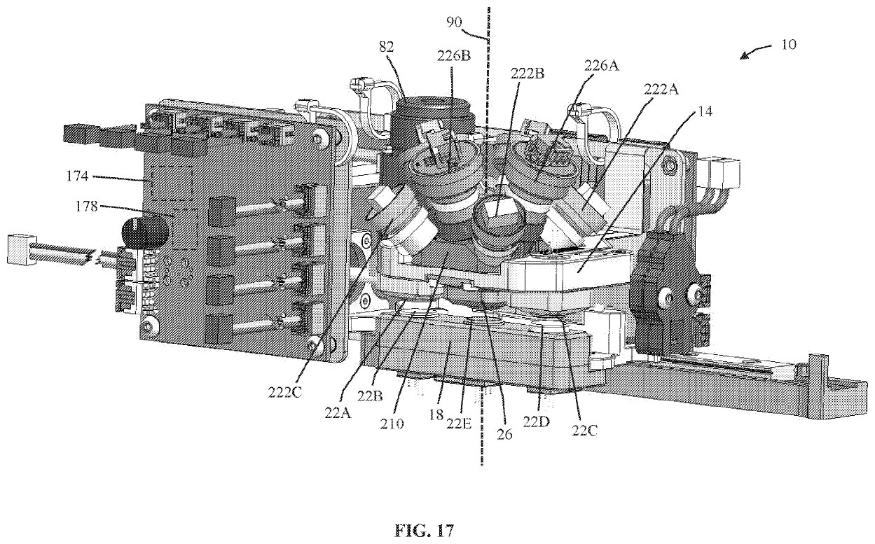

With reference to FIG. 17, a heating and optics assembly 10 is illustrated. In some embodiments, the heating and optics assembly 10 is a part of the complementary instrument for processing and analyzing a sample and/or cartridge. The assembly 10 includes a first support 14 and a second support 18 movable with respect to the first support 14. The assembly 10 further includes a plurality of heat transfer devices 22A-22E and a fluorimeter 26.

In the illustrated embodiment, a first heat transfer device 22A is coupled to the first support 14 and a second heat transfer device 22B is coupled to the second support 18 and positioned opposite the first heat transfer device 22A. The first heat transfer device 22A includes a first planar surface 30 and the second heat transfer device 22B includes a second planar surface 34 positioned opposite the first planar surface 30. In some embodiments, the first heat transfer device 22A and the second heat transfer device 22B are maintained at a first temperature. In some embodiments, the first temperature is within a range of approximately 90° C. to approximately 100° C. In some embodiments, the first temperature is approximately 95° C.

Likewise, a third heat transfer device 22C is coupled to the first support 14 and a fourth heat transfer device 22D is coupled to the second support 18 and positioned opposite the third heat transfer device 22C. The third heat transfer device 22C includes a third planar surface 38 and the fourth heat transfer device 22D includes a fourth planar surface 42 positioned opposite the third planar surface 38. In some embodiments, the third heat transfer device 22C and the fourth heat transfer device 22D are maintained at a second temperature. In some embodiments, the second temperature (of the third and fourth heat transfer devices 22C, 22D) is different than the first temperature (of the first and second heat transfer devices 22A, 22B). In some embodiments, the second temperature is within a range of approximately 60° C. to approximately 70° C. In some embodiments, the second temperature is approximately 65° C.

In the illustrated embodiment, the fluorimeter 26 is coupled to the first support 14 and is positioned between the first heat transfer device 22A and the third heat transfer device 22C. In the illustrated embodiment, the assembly 10 further includes a fifth heat transfer device 22E coupled to the second support 18 and positioned opposite the fluorimeter 25. In particular, the fifth heat transfer device 22E includes a fifth planar surface 46 positioned opposite the fluorimeter 26. The fifth heat transfer device 22E is positioned between the second heat transfer device 22B and the fourth heat transfer device 22D. In some embodiments, second, fourth, and fifth heat transfer device 22B, 22D, 22E are each biased by a biasing member 48 (e.g., a compression spring) to move relative to the second support 18 toward the first support 14.

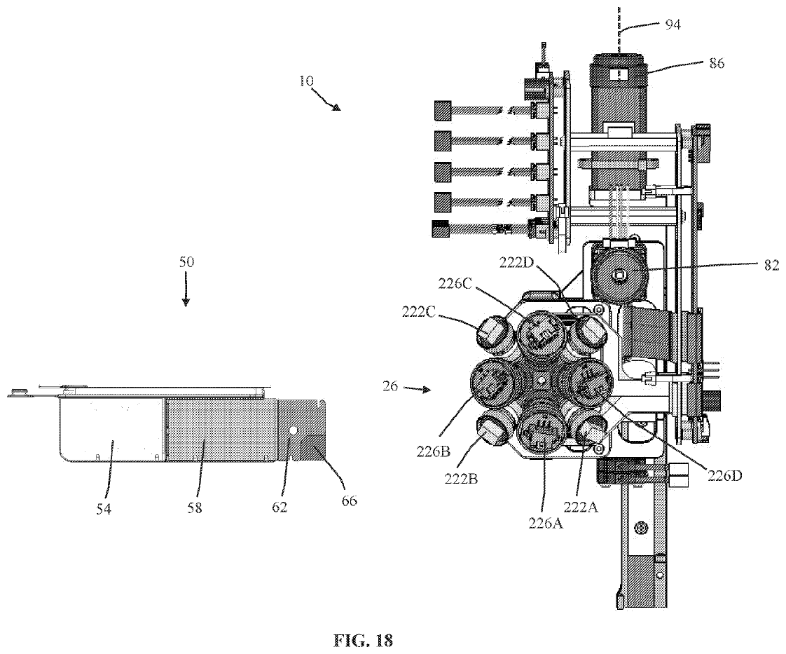

With reference to FIG. 18, a cartridge 50 for detecting levels of an analyte is illustrated. The cartridge 50 includes a storage section 54, a processing section 58, and a microfluid section 62. In the illustrated embodiment, the processing section 58 is positioned between the storage section 54 and the microfluid section 62. The cartridge 50 contains all the components and chambers needed to process and detect a target analyte. In some embodiments, the cartridge 50 is acted on by a processing apparatus that includes a servomechanism, for example, to perform operations including, but not limited to, heat transfer, liquid transfer, magnetic transfer, and optical detection. In general, the storage section 54 contains the specimen to be analyzed along with buffers and a transfer capsule used in processing; the processing section 58 is where targets are extracted, purified and bound to magnetic beads; and the microfluid section 62 is where the target or targets are detected.

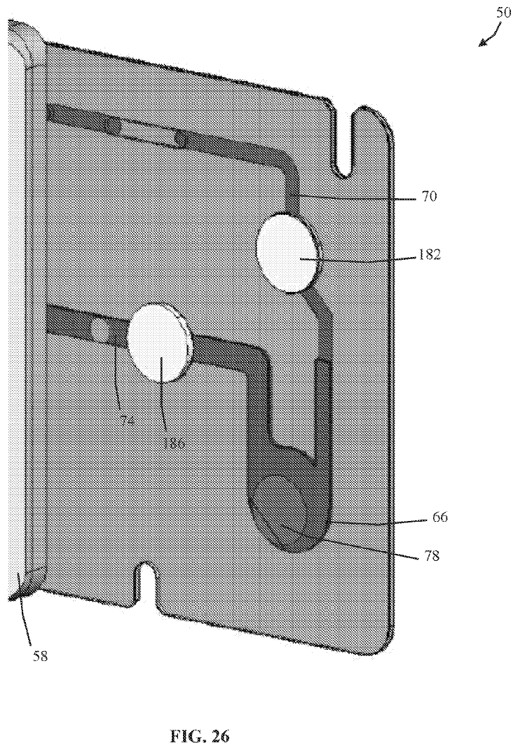

With reference to FIG. 26, the microfluid section 62 of the cartridge 50 includes a reaction chamber 66, a microfluid vent channel 70, and a microfluid inlet channel 74. The microfluid vent channel 70 is fluidly connected to the reaction chamber 66. The microfluid inlet channel 74 acts as an interface between the processing section 58 and the microfluid section 62 and facilitates easy transfer of liquids to the microfluid section 62, which has openings that are not directly accessible by a pipettor. The reaction chamber 66 is positioned between the microfluid inlet channel 74 and the microfluid vent channel 70. In some embodiments, a lyophilized master mix 78 is positioned within the reaction chamber 66 and PCR, for example, is performed therein. In the illustrated embodiment, the reaction chamber 66 is a PCR chamber.

With reference to FIG. 2, as explained in greater detail herein, the heating and optic assembly 10 is configured to receive a reaction chamber (e.g., the reaction chamber 66) between the first planar surface 30 of the first heat transfer device 22A and the second planar surface 34 of the second heat transfer device 22B to bring the reaction chamber to the first temperature. Likewise, the assembly 10 is configured to receive the reaction chamber between the third planar surface 38 and the fourth planar surface 42 of the heat transfer devices 22C, 22D to bring the reaction chamber to the second temperature.

With continued reference to FIGS. 17 and 18, the assembly 10 further includes a first actuator 82 and a second actuator 86 configured to move portions of the assembly 10 relative to the cartridge 50. The first actuator 82 is coupled to the second support 18 and is configured to move the second support 18 along a clamp axis 90 between a first position and a second position. Although the clamp axis 90 is depicted as a linear axis, in certain embodiments the clamp axis is a curve or arc of a circle. In the first position, the first planar surface 30 and the second planar surface 34 are spaced apart by a first distance; and in the second position, the first planar surface 30 and the second planar surface 34 are spaced apart by a second distance, smaller than the first distance. In some embodiments, the second distance is within a range of approximately 400 micrometers to approximately 600 micrometers. In other words, the first actuator 82 moves the second support 18 along the clamp axis 90 to clamp the cartridge 50 between the supports 14, 18. In the illustrated embodiment, the cartridge 50 is clamped between the first and second heat transfer devices 22A, 22B; between the third and fourth heat transfer devices 22C, 22D; and between the fluorimeter 26 and the fifth heat transfer device 22E.

In the illustrated embodiment, the second actuator 86 is coupled to the first support 14 and the second support 18, and the second actuator 86 is configured to move the first support 14 and the second support 18 together along a translation axis 94. Although the translation axis 94 is depicted as a linear axis, in certain embodiments the translation axis is a curve or arc of a circle. In some embodiments, the translation axis 94 is perpendicular to the clamp axis 90. In some embodiments, the translation axis 94 is vertical and the clamp axis 90 is horizontal. In other words, the second actuator 86 moves the supports 14, 18 with respect to the cartridge 50 along the translation axis 94. Movement along the translation axis 94, aligns the reaction chamber 66 of the cartridge 50 with different heat transfer devices 22A-22E and/or the fluorimeter 26. In some embodiments, the cartridge 50 remains stationary as the supports 14, 18 move relative to the cartridge 50 along the translation axis 94 and/or the clamp axis 90. In other embodiments, the supports 14, 18 remain stationary as the cartridge 50 moves relative to the supports 14, 18.

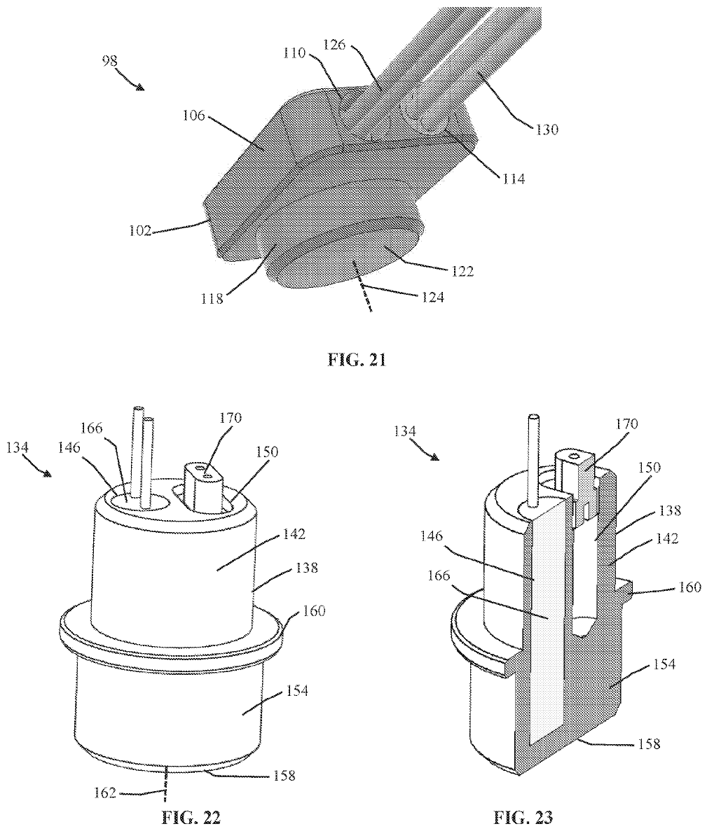

With reference to FIG. 21, a heat transfer device 98 for heating a reaction chamber (e.g., the PCR reaction chamber 66) is illustrated. In some embodiments, the heat transfer device 98 may be any of the heat transfer devices 22A-22E in the heating and optic assembly 10. The heat transfer device 98 includes a heat reservoir 102 with a base 106, a first bore 110, a second bore 114, and a heat exchanger 118 extending from the base 106. The heat exchanger 118 includes a planar surface 122 (e.g., the planar surfaces 30, 34, 38, 42, 46) that is configured to about the reaction chamber 66. In the illustrated embodiment, the heat exchanger 118 is cylindrical, and the base 106 is rectangular. In some embodiments, the heat reservoir is high thermally conductive material (e.g., Aluminum). In the illustrated embodiment, the first bore 110 and the second bore 114 are formed in the base 106 and extend perpendicular to a longitudinal axis 124 of the heat exchanger 118. In the illustrated embodiment, the first bore 110 and the second bore 114 are formed in the same surface of the base 106 and extend parallel to each other. The heat transfer device 98 further includes a heater 126 positioned within the first bore 110 and a temperature sensor 130 positioned within the second bore 114. In some embodiments, the heater 126 is an electric resistive heater.

With reference to FIGS. 22 and 23, a heat transfer device 134 for heating and/or cooling a reaction chamber (e.g., the reaction chamber 66) is illustrated. In some embodiments, the heat transfer device 134 may be any of the heat transfer devices 22A-22E in the heating and optic assembly 10. The heat transfer device 134 includes a heat reservoir 138 with a base 142, a first bore 146, a second bore 150, and a heat exchanger 154 extending from the base 142. The heat exchanger 154 includes a planar surface 158 (e.g., the planar surfaces 30, 34, 38, 42, 46) that is configured to abut the reaction chamber 66. In the illustrated embodiment, the heat exchanger 154 is cylindrical, the base 142 is cylindrical, and a flange 160 separates the two portions 142, 154. In some embodiments, a biasing member (e.g., spring 48) abuts the flange 160 to biasing the heat transfer device 134 in a direction (e.g., toward another heat transfer device mounted opposite). In the illustrated embodiment, the first bore 146 and the second bore 150 are formed in the base 142 and extend parallel to a longitudinal axis 162 of the heat exchanger 154. In the illustrated embodiment, the first bore 146 and the second bore 150 are formed in the same surface of the base 142 and extend parallel to each other. The heat transfer device 134 further includes a heater 166 positioned within the first bore 146 and a temperature sensor 170 positioned within the second bore 150. In some embodiments, the heater 166 is an electric resistive heater.

In some embodiments, the heat transfer devices 22A-22E and the assembly 10 include a processor 174 and a non-transitory memory 178. In some embodiments, the memory 178 includes instructions that when executed by the processor 174 performs closed-loop temperature control of the heat reservoir of the heat transfer devices 22A-22E. By using the temperature sensor (e.g., sensor 130, 170) as feedback and the heater (e.g., 126, 166) a controlled manipulated input, closed loop temperature control of the heat reservoir is achieved. In some embodiments, the closed-loop control is a proportional-integral-derivative (“PID”) or proportional-integral (“PI”) controller.

In some embodiments, an instrument for use in embodiments herein comprises one or more magnetic transfer elements. In some embodiments, a magnetic transfer element comprises a magnet which can be positions at various locations along the cartridge and ad various distances from the chambers/channels of the device, in order to vary the magnetic force at locations within the device. In some embodiments, the magnetic transfer element comprises a mechanical arm with a magnet located at its terminus. The distance between the magnet and the side of one or more of the device chambers (e.g., C5, C6, reaction chamber) can be varied (e.g., 1 mm, 2 mm, 3 mm, 4 mm, 5 mm, 6 mm, 7 mm, 8 mm, 9 mm, 10 mm, 11 mm, 12 mm, 13, mm, 14 mm, 15 mm, 16 mm, 17, mm, 18, mm, 19 mm, 20 mm, or more, or ranges therebetween) to apply various magnetic force to the contents of the chamber (e.g., PMPs). In some embodiments, the mechanical arm allow the magnet to be moved vertically from the top to bottom of a chamber (and from bottom to top) and laterally (e.g., to associate with a different chamber, to drag a pellet through a transfer channel, etc.

In some embodiments, an instrument comprises a fluorometer, camera, or other optical reader capable of detecting light emitted from a sample, for example, within the detection/reaction chamber of the device. In some embodiments, a fluorometer is provided that is capable of exciting a fluorophore within the detection/reaction chamber of the device and detecting the wavelengths of light emitted.

Numerous techniques for detecting the presence and/or concentration of an in a sample within the detection/reaction chamber are available. For instance, fluorescent labeling of the analyte may be used. A fluorescent label (or fluorescent probe) is generally a substance which, when stimulated by an appropriate electromagnetic signal or radiation, absorbs the radiation and emits a signal (usually radiation that is distinguishable, e.g., by wavelength, from the stimulating radiation) that persists while the stimulating radiation is continued, i.e. it fluoresces. Fluorometry involves exposing a sample containing the fluorescent label or probe to stimulating (also called excitation) radiation, such as a light source of appropriate wavelength, thereby exciting the probe and causing fluorescence. The emitted radiation is detected using an appropriate detector, such as a photodiode, photomultiplier, charge-coupled device (CCD), or the like. In some embodiments, a complementary instrument for the cartridge device comprises an appropriate detector (e.g., photodiode, photomultiplier, charge-coupled device (CCD), fluorometer, luminometer, etc.).

Fluorometers for use with fluorescent-labeled samples are known in the art. One type of fluorometer is an optical reader, such as described by Andrews et al. in U.S. Pat. No. 6,043,880; incorporated by reference in its entirety. Optical readers may be integrated within reaction chambers (e.g., thermal cyclers), so that the sample may be analyzed without removing it from the reaction chamber (e.g., without interrupting PCR). Examples of such integrated devices are described in U.S. Pat. Nos. 5,928,907, 6,015,674, 6,043,880, 6,144,448, 6,337,435, and 6,369,863; incorporated by reference in their entireties. Such combination devices are useful in various applications, as described, e.g., in U.S. Pat. Nos. 5,210,015, 5,994,056, 6,140,054, and 6,174,670; incorporated by reference in their entireties.

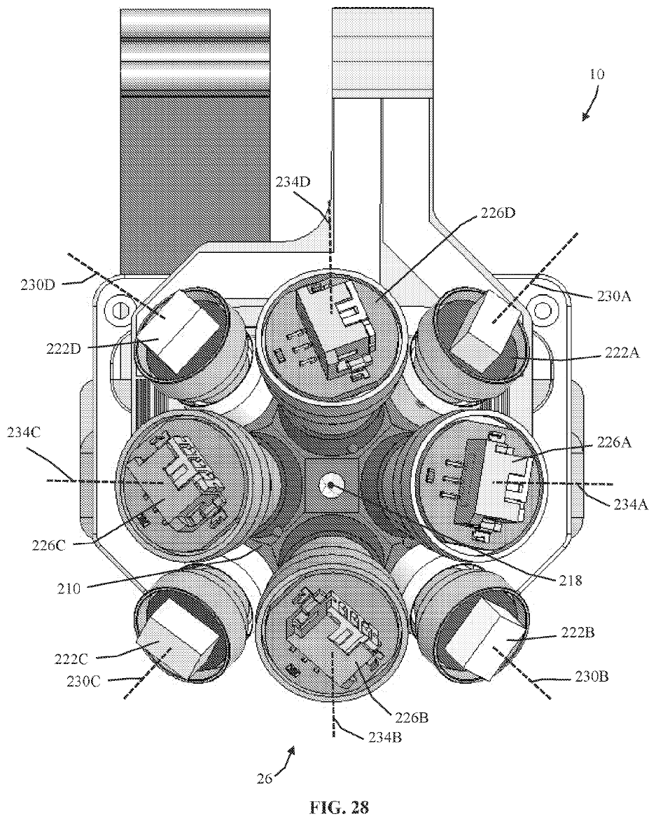

In some embodiments, systems, devices, and components thereof are provided comprise fluorimeters. In some embodiments, a complementary instrument for use with a cartridge device (e.g., as described herein) comprises a fluorimeter. In some embodiments, for example, with reference to FIGS. 28-30, the fluorimeter 26 is integrated with the heat transfer devices 22A-22E in a single assembly (e.g., the heating and optics assembly 10). As discussed herein, quantitative PCR takes a plurality of fluorescence measurements after a plurality of thermal cycles. The time to perform the PCR is therefore reduced by the heating and optics assembly 10, which is compact and can efficiently move heaters and the fluorimeter 26 relative to the cartridge 50.

With continued reference to FIGS. 28-30, the fluorimeter 26 is coupled to the first support 14. In the illustrated embodiment, the fluorimeter 26 includes a casing 210 that defines a measurement aperture 214. As detailed herein, the measurement aperture 214 is aligned with the PCR chamber 66 to take fluorescence measurements of the sample. In some embodiments, the measurement aperture 214 is configured to align with a planar surface of a PCR chamber (e.g., a front surface). The measurement aperture 214 defines a normal axis 218 that is perpendicular to the measurement aperture 214. As such, the normal axis 218 is perpendicular to the PCR chamber 66 during fluorescence measurements.

Conventional fluorescence measurements are taken at right-angles (e.g., from the edges) to minimize the excitation light collected by emission optics. However, in some embodiment, the thickness of the PCR chamber is small (e.g., less than 500 micrometers) and right-angle fluorescence readings from the edges are not feasible. In the illustrated embodiment, excitation light enters, and fluorescence is detected through the front surface of the PCR chamber 66. This allows a large fraction of the fluorescence in the chamber to be collected. Typically, this type of measurement requires the use of dichromic mirrors and/or beam splitters that reflect excitation light and pass emitted light, and results in a complicated optical system.

The fluorimeter 26 includes a plurality of light sources 222A-222D and a plurality of light detectors 226A-226D. In the illustrated embodiment, the fluorimeter 26 includes four light sources 222A, 222B, 222C, and 222D. The first light source 222A is coupled to the casing 210 along a first light source axis 230A, the second light source 222B is coupled to the casing 210 along a second light source axis 230B, the third light source 222C is coupled to the casing 210 along a third light source axis 230C, and the fourth light source 222D is coupled to the casing 210 along a fourth light source axis 230D. Each of the light source axes 230A-230D intersect the measurement aperture 214. In other embodiments a fluorimeter includes 1-10 (e.g., 1, 2, 3, 4, 5, 6, 7, 8, 9, or 10) light sources and 1-10 (e.g., 1, 2, 3, 4, 5, 6, 7, 8, 9, or 10) light source axes that are non-co-axial and intersect the measurement aperture.

In the illustrated embodiment, the fluorimeter 26 includes four light detectors 226A-226D, with one light detector corresponding to each of the light sources. The first light detector 226A is coupled to the casing 210 along a first detector axis 234A, the second light detector 226B is coupled to the casing 210 along a second detector axis 234B, the third light detector 226C is coupled to the casing 210 along a third detector axis 234C, and the fourth light detector 226D is coupled to the casing 210 along a fourth detector axis 234D. Each of the detector axes 234A-234D intersect the measurement aperture 214.

In the illustrated embodiment, the light source axes 230A-230D and the detector axes 234A-234D all intersect the measurement aperture 214. None of the light source axes 230A-230D, the detector axes 234A-234D, and the normal axis 218 are co-axial. In other words, the light source axes 230A-230D and the detector axes 234A-234D intersect each other at the measurement aperture 214 but do not otherwise overlay each other. In the illustrated embodiment, the light source axes 230A-230D and the detector axes 234A-234D are positioned circumferentially around the normal axis 218 (FIG. 28). In some embodiments, the light sources 222A-222D and the detectors 226A-226D alternative in the circumferential direction. In some embodiments, the detector axis is adjacent to the LED axis. For example, in the illustrated embodiment, the first light source 222A is positioned circumferentially between the first detector 226A and the fourth detector 226D. Likewise, the first detector 222A is positioned circumferentially between the first light source 222A and the second light source 222B.

The first light source 222A emits a first excitation light along the first light source axis 230A and the first excitation light is reflected at the measurement aperture 214 away from the first detector axis 234A. In other words, the excitation light beam enters the PCR chamber 66 along one optical axis (e.g., the first light source axis 230A), and emitted light is collected along a separate optical axis (e.g., the first detector axis 234A). The angles between the axes 230A, 234A and the surface of the PCR chamber 66 are selected so that excitation light is reflected away from the emission optical axis. This allows for multiple pairs of excitation and emission optics to measure fluorescence in the same chamber, without using fiber optics, dichroic mirror, or moving filter modules. In the illustrated embodiment, the fluorimeter 26 advantageously does not include a dichroic mirror or a beam splitter. The fluorimeter 26 is small, lightweight, and compact, such that the fluorimeter 26 is integrated with the heat transfer devices 22A-22E used for target amplification.

Together, the four light sources 222A-222D and the four light detectors 226A-226D create four channels of fluorescence detection. In some embodiments, the fluorimeter 26 includes at least four channels of fluorescence detection. For example, the first excitation light from the first light source 222A has a first spectrum (e.g., a first spectral power distribution) and the first light detector 226A measures a first fluorescence of the sample in response to the first excitation light (e.g., the first channel). Likewise, a second excitation light is emitted from the second light source 222B with a second spectrum (e.g., a second spectral power distribution) and the second light detector 226B measures fluorescence of the sample in response to the second excitation light. Advantageously, more light source/detector pairs (e.g., channels) can be placed around a central axis (e.g., the normal axis 218), without significantly reducing signal intensity, by reducing the diameters of the lenses while keeping approximately the same numerical apertures.

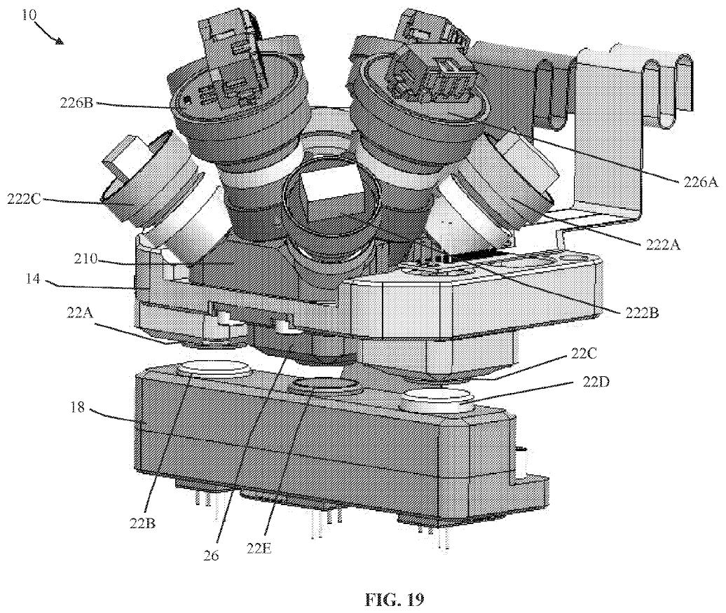

With reference to FIG. 19, the first light source 222A (which is representative of the structure of each light source) includes a light emitter 238, a lens 242, and a wavelength selecting filter 246. In some embodiments, the light emitter 238 is a light emitting diode (LED). For some fluorophores and LEDs, the wavelength selecting filter can be omitted. The first excitation light hits the reaction chamber 66 and is reflected away from the first detector 226A. In other words, stray light from the first light source 222A is reflected away from the first detector 226A because the angle of incidence of the first light source axis 230A is greater than the angle of incidence of the first detector axis 234A. Fluorescence in response to the first excitation light is detected by the first light detector 226A. In some embodiments, the intensity of the first excitation light is many orders of magnitude greater than the emitted fluorescence light.

Fluorescent light can be emitted from the reaction chamber 66 at longer wavelengths. The first light detector 226A (which is representative of the structure of each light detector) includes a first lens 150, a filter 254 (e.g., a wavelength selecting filter), a second lens 258, and a solid-state detector 262. For some fluorophores, second lens can be omitted because the light is sufficiently collimated by the first lens. Light impacting the solid-state detector 262 is converted to an electrical signal measurement detected and stored by the processor 174. In some embodiments, the non-transitory memory 178 includes instructions that, when executed by the processor 174, store four hundred (400) analog to digital readings by the first detector 226A over a 100 millisecond time period. To minimize ambient noise due to visible light, radiated and conducted AC noise, four hundred analog-to-digital readings are made over a fixed 100 millisecond time period. This time period is the length of precisely six cycles at 60 Hz and five cycles at 50 Hz such that the variations in the signal due to the AC power sources in different countries are averaged out.

### IV. Methods, Kits, and Systems

In some embodiments, provided herein are methods of sample processing and analyte detection/quantification performed using the devices and systems described herein. In some embodiments, provided herein are systems, kits, and methods for preparing target nucleic acids in a biological sample for subsequent analysis.

The devices herein find use in processing a variety of sample types (e.g., biological (e.g., tissue, blood, blood products, saliva, etc.) environmental (e.g., soil or water sample), research (e.g., cell culture, in vitro sample, etc.), etc.), detecting a variety of analytes (e.g., nucleic acids, small molecules, peptides, proteins, etc.), with a variety of detection reagents (e.g., primers, probes, antibodies, etc.), and through a variety of detection techniques (e.g., PCR, fluorescence, immunoassay).

In some embodiments, reagents used in the methods/kits/systems herein are provided in a dried (e.g., lyophilized discs, pellets, etc.) or concentrated (e.g., liquid, gel, etc.) forms. In some embodiments, the reagents used in the methods/kits/systems herein include components for cell lysis (e.g., detergents (e.g., SDS), etc.), components for protein digestion (e.g., proteinase K, etc.), nucleic acid capture probes (e.g., a hybridization sequence linked to a capture moiety (e.g., biotin, etc.)), capture-agent-coated magnetic beads (e.g., streptavidin-coated beads), amplification reagents (e.g., primers, nucleotides, magnesium, etc.), detection reagents (e.g., fluorescent labels), etc.

In some embodiments, methods utilize three dried or concentrated reagent compositions (e.g., lyophilized discs, pellets, etc.; concentrated liquids, gels, etc.) for target nucleic acid capture/isolation/purification, and optionally one additional dried or concentrated reagent composition for target nucleic acid amplification/detection. In some embodiments, the three dried or concentrated reagent compositions for target nucleic acid capture/isolation/purification are a lysis reagent (and/or a protein digestion reagent), a capture reagent, and capture-agent-coated magnetic beads. In some embodiments, methods comprise one or more (e.g., all) of the steps of (a) combining the biological sample with a lysis reagent capable of digesting cell membranes and degrading proteins and allowing the lysis reagent to digest cell membranes and degrade proteins to generate a lysate, wherein the biological sample comprises nucleic acid; (b) combining the lysate with a capture reagent, wherein the capture reagent comprises a nucleic acid probe tethered to a capture moiety; (c) allowing the nucleic acid probe to hybridize to the nucleic acids of the biological sample to generate a probe-bound nucleic acid solution; (d) combining the probe-bound nucleic acid with capture-agent-coated magnetic beads; (e) allowing the capture agent to bind to the capture moiety to generate a bead-captured nucleic acid suspension; (f) isolating bead-captured nucleic acids within the bead-captured nucleic acid suspension by exposing a portion of the bead-captured nucleic acid suspension to a magnetic field; and (g) separating the isolated, bead-captured nucleic acids from a liquid portion of the bead-captured nucleic acid suspension.

In some embodiments, methods utilize two dried or concentrated reagent compositions (e.g., lyophilized discs, pellets, etc.; concentrated liquids, gels, etc.) for target nucleic acid capture/isolation/purification, and optionally one additional dried or concentrated reagent composition for target nucleic acid amplification/detection. In some embodiments, the two dried or concentrated reagent compositions for target nucleic acid capture/isolation/purification are a lysis/capture reagent and capture-agent-coated magnetic beads. In some embodiments, methods comprise one or more (e.g., all) of the steps of: (a) combining the biological sample with a lysis reagent and a capture reagent, wherein the lysis reagent comprises components capable of digesting cell membranes and degrading cellular proteins, wherein the capture reagent comprises a nucleic acid probe tethered to a capture moiety, and wherein the biological sample comprises nucleic acid; (b) allowing the lysis reagent to digest cell membranes and degrade proteins to generate a lysate; (c) allowing the nucleic acid probe to hybridize to the nucleic acids of the biological sample to generate a probe-bound nucleic acid solution; (d) combining the probe-bound nucleic acid with capture-agent-coated magnetic beads; (e) allowing the capture agent to bind to the capture moiety to generate a bead-captured nucleic acid suspension; (f) isolating bead-captured nucleic acids within the bead-captured nucleic acid suspension by exposing a portion of the bead-captured nucleic acid suspension to a magnetic field; and (g) separating the isolated, bead-captured nucleic acids from a liquid portion of the bead-captured nucleic acid suspension.

Certain embodiments herein do not comprise a centrifugation step, do not comprise a filtration step, and/or do not comprise precipitation of nucleic acids. In some embodiments, the nucleic acids for the biological sample, including the target nucleic acids, are not separated from contaminants of the biological sample or excess reagents (e.g., unbound probes) during the lysis, digestion, probe hybridization, and/or capture (e.g., binding of the bead-bound capture agent to the probe-bound capture moiety) steps.

Embodiments herein require a sample that comprises nucleic acids (or is suspected to contain nucleic acids). Suitable samples contain nucleic acids and are therefore referred to herein as biological samples. Biological samples may be or any source or origin, may be obtained from nature or be specimens generated in a lab. Biological samples may be obtained from animals (including humans) and encompass fluids, solids, tissues, and gases. Some biological samples include blood products, such as plasma, serum, stool, urine, and the like. Samples may be of environmental origin and may include environmental material such as surface matter, soil, mud, sludge, biofilms, water, and industrial samples. In some preferred embodiments, a sample comprises nucleic acid from a pathogen (e.g., virus, bacteria, fungi, protozoa, worms, etc.). In other embodiments, samples comprise nucleic acid from a subject (e.g., human, non-human primate, livestock, wild animal, etc.). Any sample containing any type of nucleic acid may find use in embodiments herein. Exemplary samples provided herein are not to be construed as limiting the sample types applicable to the present invention.

In some embodiments, a sample is added to a chamber of a device herein, for example, using an open top of the chamber, and the chamber is seals (e.g., with a cap). In the case of a liquid sample, the sample may be injected into the chamber (e.g., an empty chamber, a chamber comprising an appropriate buffer, etc.). In the case of a solid sample, or a sample on a solid medium (e.g., wipe, swap tip, etc.), the solid may be inserted into the chamber, and the sample is dissolved into a liquid in the chamber.

In some embodiments, a sample is digested in a chamber of a device herein. In some embodiments, depending upon the sample type and the target analyte, appropriate processing reagents are used. For example, the reagents may include protein precipitation reagents (e.g., acetonitrile, methanol, or perchloric acid), cell lysis reagents (e.g., zinc sulfate, a strong acid, an enzyme digestion with lysozymes, cellulases, proteases, detergents including, without limitation, non-ionic, zwitterionic, anionic, and cationic detergents, protein digestion reagents (e.g., serine proteases such as trypsin, threonine, cysteine, lysine, arginine, or aspartate proteases, metalloproteases, chymotrypsin, glutamic acid proteases, lys-c, glu-c, and chemotrypsin), internal standards (e.g., stabile isotope labeled analytes, heavy isotope labeled peptides, non-native peptides or analytes, structurally similar analogs, chemically similar analogs), antibiotics (for microbiological antibiotic susceptibility testing, or “AST”), protein stabilization agents, including buffers, chaotropic agents, or denaturants, calibration standards, and controls. According to various embodiments, one or more of the reagents may be pre-mixed to form a combined reagent mixture specific for a particular assay or panel of assays. In some embodiments, reagents are included in a buffer or in a lyophilized reagent pellet. In some embodiments, a sample is exposed to the appropriate processing reagents and condition (e.g., temperature) to breakdown components of the sample that interfere with the assay (e.g., cell lysis, protein degradation, etc.).

In some embodiments, a biological sample is combined with a reagent described herein (e.g., lysis and/or digestion reagent) to initiate the steps of a method herein. In other embodiments, a liquid buffer solution is added to the biological sample (or the sample is added to a liquid buffer solution) in order to dilute the sample, bring the sample into a sufficient volume for handling, and/or to extract the sample from a substrate (e.g., swab, collection vial, etc.). In some embodiments, a substrate comprising a biological sample or a biological sample itself is added to a liquid buffer (e.g., in a tube, well, chamber, etc.) to initiate the methods herein.

In some embodiments, a biologic sample (comprising nucleic acid) alone or in an appropriate buffer is treated with a lysis and/or digestion reagent. In some embodiments, the lysis and/or digestion reagent is a compound reagent (i.e., comprising two or more individual component reagents). In some embodiments, the lysis and/or digestion reagent comprises component reagents for degrading a cell membrane (e.g., of a bacteria or eukaryote). In some embodiments, the lysis and/or digestion reagent comprises component reagents for digesting proteins within the sample (e.g., cellular proteins, viral proteins, etc.).). In some embodiments, the lysis and/or digestion reagent comprises component reagents suitable for releasing nucleic acids from cells and/or viruses. Component reagents in a lysis and/or digestion reagent may include one or more enzymes configured to reduce (e.g., denature) proteins (e.g., proteinases, proteases (e.g., pronase), trypsin, proteinase K, phage lytic enzymes (e.g., PlyGBS)), lysozymes (e.g., a modified lysozyme such as ReadyLyse), cell specific enzymes (e.g., mutanolysin for lysing group B streptococci)). Other enzymes present in a lysis and/or digestion reagent may include lysostaphin, zymolase, cellulase, mutanolysin, glycanases, etc. In some embodiments, component reagents in a lysis and/or digestion reagent comprise one or more chemical cell lysis reagents, such as Triton-X, guanidinium salt, or SDS. In some embodiments, the lysis and/or digestion reagent may comprise various salts (e.g., NaCl, MgCl₂, etc.), buffers (e.g., Tris, MOPS, MES, etc.), or other components. In particular embodiments, a lysis regent comprises proteinase K. In other embodiments, a lysis regent comprises proteinase K and SDS. In some embodiments, the lysis reagent comprises proteinase K (e.g., 1 U, 2 U, 5 U, 10 U, 15 U, 20 U, 25 U, 30 U, 40 U, 50 U, or more, or ranges therebetween), CaCl₂) (e.g., 1 mM, 2 mM, 3 mM, 4 mM, 5 mM, 6 mM, 7 mM, 8 mM, 9 mM, 10 mM, 15 mM, 20 mM, 30 mM, or more or ranges therebetween), and/or HEPES e.g., 1 mM, 2 mM, 3 mM, 4 mM, 5 mM, 6 mM, 7 mM, 8 mM, 9 mM, 10 mM, 15 mM, 20 mM, 30 mM, or more or ranges therebetween). In some embodiments, the lysis reagent comprises 15 U proteinase K, 5 mM CaCl₂), and 5 mM HEPES.

In some embodiments, the lysis and/or digestion reagent is a dry reagent (e.g., lyophilized disc or pellet), and the dry lysis reagent is resuspended upon combination with the biological sample and/or a buffer solution. In other embodiments, the lysis and/or digestion reagent is a concentrated liquid (or gel), and is diluted in the biological sample and/or a buffer solution. A concentrated lysis reagent may be present at a concentration of 10×, 20×, 50×, 100 C, 200×, 500×, or greater, compared to the 1X working concentration after dilution into the biological sample and/or a buffer solution.

In some embodiments, treatment of a sample to generate a lysate may include physical processes in addition to the chemical regents and enzymes above or understood in the field. For example, in some embodiments, a sample is heated to assist in lysis (e.g., >60° C., >65° C., >70° C., >75° C., >80° C., >85° C., >90° C., >95° C., etc.). In some embodiments, a sample is heated (e.g., 90-100° C.) following lysis to inactivate one or more of the enzymes employed for lysis. In some embodiments, freeze/thaw is employed to assist with lysis. In some embodiments, mechanical means such as French press, milling, sonication, etc. are utilized for lysis. In some embodiments, methods herein do not employ mechanical means of lysing cells or viruses.

In some embodiments, upon release of the nucleic acid from a cell, virus, etc., the lysate is combined with a capture reagent. In some embodiments, a capture reagent comprises a nucleic acid probe that comprises a hybridization sequence and a capture moiety.

In some embodiments, after appropriate processing of the sample to liberate the target analyte (e.g., cell lysis, digestion of contaminant components, etc.), a capture agent (e.g., sequence specific capture probe) is used to bind (capture) the target analyte. In some embodiments, a capture agent comprises a target binding moiety and a handle or affinity moiety. The target binding moiety may be any molecular entity (e.g., nucleic acid probe, antibody or antibody fragment, target-specific ligand, etc.) capable of stably binding to the target analyte. The binding moiety may be, for example, a nucleic acid probe sequence, effective to hybridize to a target nucleic acid sequence, or an antibody or functional fragment thereof, effective to bind a target protein or other analyte. Any binding moiety of any desired specificity may be used. The handle or affinity moiety is one element of an affinity pair that can be used to capture the analyte when bound to the capture agent. Immunoreactive specific binding members include antigens or antigen fragments and antibodies or functional antibody fragments. Other specific binding pairs include biotin and avidin, carbohydrates and lectins, complementary nucleotide sequences, effector and receptor molecules, cofactors and enzymes, enzyme inhibitors and enzymes, and the like. In some embodiments, a binding member is attached to a solid phase support, such as a plurality of paramagnetic particles, in order to extract the analyte from a sample containing non-target components. In preferred embodiments, sequence specific capture probes comprising a biotin moiety or another affinity handle are hybridized to target analyte nucleic acids. Subsequently, the capture-probe-bound nucleic acid is captured onto paramagnetic particles (PMPs) comprising streptavidin or another affinity agent capable of binding to the handle. In some embodiments, washing of the pelleted PMPs provides for removal of contaminants from the captured target analyte. In the case of non-nucleic acid targets, other suitable means of capture are understood.

In some embodiments, the hybridization sequence is a polynucleotide sequence that is complementary to all or a portion of a target sequence in the target nucleic acid. In some embodiments, the hybridization sequence is sufficiently complementary to the target sequence to allow for hybridization of the probe to the target nucleic acid under the conditions of the methods herein. In some embodiments, a hybridization sequence is at least 70% complementary to a target sequence (e.g., 70%, 75%, 80%, 85%, 90%, 95%, 99%, 100%).

In some embodiments, a capture moiety is a chemical group that is capable of being stably bound by a capture agent under the conditions of the methods herein. In some embodiments, the capture moiety is biotin (and the capture agent is streptavidin). In other embodiments, the capture moiety is a haloalkane (and the capture agent is HALOTAG®, Promega), an alkyne (and the capture agent is an azide), etc.

In some embodiments, the capture reagent is a dry reagent (e.g., lyophilized disc or pellet), and the dry capture reagent is resuspended upon combination with the lysate and/or a buffer solution. In other embodiments, the capture reagent is a concentrated liquid (or gel), and is diluted in the lysate and/or a buffer solution. A concentrated capture reagent may be present at a concentration of 10×, 20×, 50×, 100 C, 200×, 500×, or greater, compared to the 1X working concentration after dilution into the lysate and/or a buffer solution.

In some embodiments, the lysate is added to the concentrated or dry capture reagent. In some embodiments, the lysate and the capture reagent are mixed (e.g., stirring, aspiration, etc.). In some embodiments, the sufficient time and conditions are provided to allow hybridization of the probes to the target nucleic acid (e.g., 30 seconds, 1 minute, 2 minutes, 3 minutes, 4 minutes, 5 minutes, or more, or ranges therebetween). In some embodiments, the capture reagent and the lysate are incubated at a temperature sufficient to promote specific hybridization of the hybridizations sequence to the target sequence (e.g., 60, 61° C., 62° C., 63° C., 64° C., 65° C., 66° C., 67° C. 68° C. 69° C., 70° C., 71° C., 72° C., 73° C., or ranges therebetween). In some embodiments, the sequence of the hybridization sequence, the degree of complementarity to the target sequence, and the conditions and temperatures used discourage non-specific hybridization of the probe.

The nucleic acid probes may also comprise other nucleic acid elements that find use in the methods herein. For example, a probe may comprise a primer binding site (for subsequent amplification of the target nucleic acid), a linker region (for attachment of the capture moiety), etc.

In some embodiments, a combined lysis/capture reagent is employed in the methods herein. Such a reagent comprises a lysis component and a capture component. In some embodiments, the lysis component is consistent with the lysis reagents described above (e.g., containing SDS and proteinase K). In some embodiments, the capture component is consistent with the capture reagents described above (e.g., containing nucleic acid probes tethered to a capture moiety). In such embodiments, the biological sample (alone or in buffer) is combined with the lysis/capture reagent and exposed to conditions suitable for lysis/digestion followed by conditions appropriate for probe hybridization. In some embodiments, changing from lysis to hybridization condition includes deactivating digestion/lysis enzymes (e.g., exposure to high temperatures) and exposure to hybridization temperatures. In some embodiments, lysed material is not removed prior to hybridization, whether separate or combined lysis and capture reagents are used.

In some embodiments, following hybridization of the capture probe to the target nucleic acid, the probe-bound nucleic acid is combined with capture-agent-coated magnetic beads. In some embodiments, contaminating species or other components of the lysate and probe-containing mixture are not removed prior to addition with the beads. In some embodiments, the probe-bound nucleic acid and the capture-agent-coated magnetic beads are mixed within the suspension. In some embodiments, elevated temperature (e.g., (e.g., 70° C., 71° C., 72° C., 73° C., 74° C., 75° C., 76° C., 77° C. 78° C. 79° C., 80° C., or ranges therebetween). is used to facilitate resuspension of the beads and mixing

In some embodiments, the capture-agent-coated magnetic beads are a dry reagent (e.g., lyophilized disc or pellet), and the dry capture-agent-coated magnetic beads are resuspended upon combination with the probe-bound nucleic acid mixture and/or a buffer solution. In other embodiments, the capture-agent-coated magnetic beads are a concentrated liquid (or gel), and are diluted in the probe-bound nucleic acid mixture and/or a buffer solution. Concentrated capture-agent-coated magnetic beads may be present at a concentration of 10×, 20×, 50×, 100 C, 200×, 500×, or greater, compared to the 1X working concentration after dilution into the probe-bound nucleic acid mixture and/or a buffer solution.

Upon combination of the capture-agent-coated magnetic beads and the probe-bound nucleic acid, the suspension is incubated at a temperature to facilitate binding of the capture agent to the capture moiety (e.g., 60, 61° C., 62° C., 63° C., 64° C., 65° C., 66° C., 67° C. 68° C. 69° C., 70° C., 71° C., 72° C., 73° C., or ranges therebetween).

In some embodiments, once the cells or viruses are lysed, the hybridization sequence of the probe is bound to the target nucleic acid, and the capture agent is bound to the capture moiety, thereby forming a target nucleic acid/capture probe/magnetic bead complex; contaminants, unused reagents, and/or non-essential components are removed for the first time in the methods herein. In some embodiments, a magnetic field is applied to the magnetic beads and the beads and all components bound thereto (capture probes and target nucleic acid) are separated from the unbound components, reagents, and contaminants of the liquid portion of the suspension. Various techniques are available for separating the beads from the liquid and unbound components for the suspension. In some embodiments, the magnetic field is head stable and the liquid is withdrawn from the vessel containing the suspension. The liquid may be removed by any suitable means, including pipetting, inversion of the vessel, by microfluidics, etc. In other embodiments, the magnetic field is moved to drag or stream the beads across the liquid/air interface. “Dragging” the magnetic beads across the liquid/air interface comprises positioning the magnetic field to create a pellet of the magnetic beads. The magnetic field is then moved through the liquid, sic that the pellet moves with the magnetic field. The magnetic field moves across the liquid/air interface, thereby removing the beads from the liquid. In “dragging,” the magnet is continuously positioned over a magnetically-induced pellet, as the pellet is moved across the liquid/air interface. “Streaming” the magnetic beads across the liquid/air interface similarly comprises positioning the magnetic field to create a pellet of the magnetic beads. The pellet is moved immediately adjacent to the liquid/air interface. The magnetic field is then temporarily reduced or eliminated, and then reestablished on the opposite side of the liquid/air interface. The magnetic field pulls the beads across the interface. Streaming the PMPs across the liquid/air interface, rather than dragging (e.g., with the magnet continuously positioned over a magnetically-induced pellet of PMPs), reduces elongation of the liquid/air interface and reduces the amount of undesired liquid carried-over with the PMPs into the air gap. In some embodiments, streaming is achieved by: (i) creating a magnetic field to pull the beads into a pellet (e.g., on a surface of the vessel containing the suspension), (ii) moving the magnetic field to bring the pellet near or adjacent to the air/liquid interface, (iii) reducing or eliminating the magnetic field experienced by the beads (e.g., by lifting the magnet away from the vessel, (iv) re-establishing the magnetic field on the opposite (air side) of the liquid/air interface, and (v) allowing the beads pelleted within the liquid to stream out of the liquid into the air. In some embodiments, by streaming the bead across the interface, rather than dragging the entire pellet across, less contaminating liquid is carried over with the beads. However, any methods of separating the beads from the liquid and unbound contaminants finds use in embodiments herein.

In some embodiments, upon initial isolation of the bead-captured target nucleic acid from the majority of the unbound lysate components, capture reagents, buffer, etc., the isolated beads and bead-captured target nucleic acid is subjected to one or more wash steps. A typical wash step comprises combining a wash buffer with the isolated beads and bead-captured target nucleic acid, mixing the beads and buffer to allow residual contaminants and unbound reagents to wash off the beads, target nucleic acid, probe, etc., and then repeating the process of isolating the beads from the liquid (e.g., with the methods steps described above). In some embodiments, one to five wash steps are performed (e.g., 1, 2, 3, 4, 5). In some embodiments, washing the beads comprises the steps of combining the isolated bead-captured nucleic acid with a wash buffer; resuspending the bead-captured nucleic acid in the wash buffer; isolating the bead-captured nucleic acids within the wash buffer by exposing a portion of the bead-captured nucleic acids to a magnetic field; and separating the isolated, bead-captured nucleic acids from the wash buffer.

In addition to preparing biological samples for analysis by the methods herein, buffer solutions may be utilized in the methods herein for washing bead-bound nucleic acids, resuspending regents, transferring components of the steps herein, performing amplification reactions, etc. Buffer solutions used in certain embodiments herein may include one or more of NaCl, Mg Cl₂, EDTA, sucrose, tergitol, BME, Bis Tris buffer, Tris buffer, sorbitol, dextran, polyvinylsulfonic acid, Lithium dodecylsulfate, bovine serum albumin, triton X-100, citric acid, DTT, CHAPS, NaOH, LiCl, MES buffer, phosphate buffer, etc. Buffer solutions may contain one or more salts, surfactants, detergents, anti-foaming agents, etc. Suitable combinations, as well as other components of buffer solutions for the handling, lysis, and/or digestion of biological samples, and/or for nucleic acid hybridization, capture (e.g., biotin/streptavidin binding), washing, resuspension, nucleic acid amplification, and/or fluorescence detection will be understood in the field and may find use in embodiments herein. In particular embodiments, the buffer solution comprises Lithium dodecylsulfate (e.g., 0.1%, 0.2%, 0.5%, 1%, 1.5%, 2%, 3%, 4%, 5%, or more, or ranges therebetween), EDTA (e.g., 5 mM, 10 mM, 15 mM, 20 mM, 25 mM, 30 mM, 35 mM, 40 mM, 50 mM, 60 mM, or more, or ranges therebetween), LiCl₂ (e.g., 50 mM, 100 mM, 150 mM, 200 mM, 250 mM, 300 mM, 350 mM, 400 mM, 500 mM, or more, or ranges therebetween), anti-foam agent (e.g., HYDROTECH, Bio-rad) (e.g., 0.1%, 0.2%, 0.5%, 1%, 1.5%, 2%, 3%, 4%, 5%, or more, or ranges therebetween), SDS (e.g., 0.1%, 0.2%, 0.5%, 1%, 1.5%, 2%, 3%, 4%, 5%, or more, or ranges therebetween), and/or Tris, pH 7.5-8.5 (e.g., pH 8.0) (e.g., 5 mM, 10 mM, 15 mM, 20 mM, 25 mM, 30 mM, 35 mM, 40 mM, 50 mM, 60 mM, or more, or ranges therebetween). In particular embodiments, the buffer solution comprises 1% Lithium dodecylsulfate, 30 mM EDTA, 300 mM LiCl₂, 1% (v/v) HYDROTECH (Bio-rad), 1% SDS, and 30 mM Tris, pH 8.0. In some embodiments, a buffer comprises 1% SDS, and 30 mM Tris, pH 8.0.

In some embodiments, following sufficient washing of the beads and target nucleic acid, the isolated bead-captured nucleic acid is resuspended in a resuspension buffer. In some embodiments, the same buffer is used for washing and resuspension. In some embodiments, a wash/resuspension buffer comprises glycerol (e.g., 1%, 2%, 5%, 10%, 15%, 20%, or more, or ranges therebetween), Tris (pH 7.5-8.5) (10 mM 20 mM, 50 mM 100 mM 150 mM, 200 mM, or more, or ranges therebetween), bicine (pH 7.5-8.5) (10 mM 20 mM, 50 mM 100 mM 150 mM, 200 mM, or more, or ranges therebetween), potassium glutamate (10 mM 20 mM, 50 mM 100 mM 150 mM, 200 mM, or more, or ranges therebetween), MnCl₂ or MgCl₂ (e.g., 0.1 mM, 0.2 mM, 0.5 mM, 1 mM, 2 mM, 3 mM, 4 mM, 5 mM, 10 mM, or more or ranges therebetween), Tween 20 (e.g., 0.01%, 0.02%, 0.03%, 0.04%, 0.05%, 0.06%, 0.07%, 0.08%, 0.09%, 0.1%, or more, or ranges therebetween), and/or HYDROTECH anti-foam agent (BioRad) (e.g., 0.05%, 0.1%, 0.2%, 0.3%, 0.4%, 0.5%, or more, or ranges therebetween). In some embodiments, for RNA applications, a buffer comprises 10% (v/v) glycerol, 100 mM Tris pH 8.0, 62.4 mM bicine pH 8.0, 65 mM potassium glutamate, 3 mM MnCl₂, 0.04% Tween 20, and 0.2% HYDROTECH anti-foam agent (BioRad). In some embodiments, for RNA applications, a buffer comprises 10% (v/v) glycerol, 100 mM Tris pH 8.0, 62.4 mM bicine pH 8.0, 65 mM potassium glutamate, 3 mM MgCl₂, 0.04% Tween 20, and 0.2% HYDROTECH anti-foam agent (BioRad).

In some embodiments, resuspension comprises combining the isolated bead-captured nucleic acid with a resuspension buffer; and resuspending the bead-captured nucleic acid in the resuspension buffer to generate a bead-captured nucleic acid resuspension.

In some embodiments, the isolated bead-captured nucleic acid or the bead-captured nucleic acid resuspension is in proper condition for amplification, analysis, and/or detection of target nucleic acid. In some embodiments, methods herein comprise combining the isolated bead-captured nucleic acid resuspension with analysis reagents. In some embodiments, analysis reagents comprise reagents for amplification of target nucleic acids, reagents for detection or quantification of target nucleic acids, reagents for sequencing target nucleic acids, etc.

In some embodiments, detection of the analyte is performed with the target analyte bound to the capture probe. In some embodiments, detection of the analyte is performed with the analyte displayed on a solid substrate (e.g., PMP). Detecting and/or quantitating the analyte can be performed by a variety of methods.

The presence or amount of a nucleic acid analyte can be determined with several methods well-known in the art. In some embodiments, quantification is absolute, i.e. relating to a specific number of target analytes, or relative, i.e. measured in arbitrary normalized units. Methods allowing for absolute or relative quantification are well known in the art, e.g., quantitative PCR methods are methods for relative quantification; if a calibration curve is incorporated in such an assay, the relative quantification can be used to obtain an absolute quantification. Other methods known are, e.g. nucleic acid sequence-based amplification (NASBA) or the Branched DNA Signal Amplification Assay. The amount of a nucleic acid analyte could be determined by a sequencing or PCR technique (e.g., fluorescence-based real-time PCR), many examples of which are understood in the field.

The presence or amount of a peptide or polypeptide analyte can be determined with various methods well-known in the art. Direct measuring relates to measuring the amount of the peptide or polypeptide based on a signal which is obtained from the peptide or polypeptide itself and the intensity of which directly correlates with the number of molecules of the peptide present in the sample. Such a signal—sometimes referred to as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products. Determining the amount of a peptide or polypeptide can be achieved by any known means for determining the amount of a peptide in a sample. Said means include immunoassay and/or immunohistochemistry methods which may utilize labeled molecules (e.g., antibodies and antibody fragments) in various sandwich, competition, or other assay formats. Said assays will develop a signal which is indicative for the presence or absence of the peptide or polypeptide.

In some embodiments, the analysis reagents are a dry reagent (e.g., lyophilized disc or pellet), and the dry analysis reagents are resuspended upon combination with the bead-captured nucleic acid resuspension and/or a buffer solution. In other embodiments, the analysis reagents are a concentrated liquid (or gel), and are diluted in the bead-captured nucleic acid resuspension and/or a buffer solution. Concentrated analysis reagents may be present at a concentration of 10×, 20×, 50×, 100 C, 200×, 500×, or greater, compared to the 1X working concentration after dilution into the bead-captured nucleic acid resuspension and/or a buffer solution.

In some embodiments, the methods herein utilize any suitable technique for the analysis, detection, quantification, sequencing, etc. of target nucleic acids. Accordingly, the methods herein may utilize and/or systems/kits herein may comprise, any components/reagents necessary for detection, quantification, sequencing, etc. of target nucleic acids by techniques understood in the art. For example, analysis reagents may comprise primers (e.g., fluorescently-labelled primers), probes, nucleotides, salts, and any other reagents understood in the field to be useful for known nucleic acid analysis techniques.

In some embodiments, analysis of target nucleic acids comprises amplification of target sequence(s). Known methods of nucleic acid amplification find use in the methods herein, and known reagents for performing such amplification techniques find use in the kits/system herein. For example, methods herein may utilize an amplification technology such as polymerase chain reaction (PCR), real-time PCR, probe hydrolysis PCR, digital PCR, reverse transcription PCR, isothermal amplification, nucleic acid sequence-based amplification (NASBA), ligase chain reaction, transcription mediated amplification, etc.

Embodiments herein include various reagents used to carry out amplification reactions, including, but not limited to, PCR reactions. Such PCR reactions and other amplification/detection/quantification techniques may be performed with any suitable analysis reagents, as described herein. DNA polymerases that can be used in accordance with these embodiments include, but are not limited to, any polymerase capable of replicating a DNA molecule. In some embodiments, DNA polymerases are thermostable polymerases, which are especially useful in PCR applications. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus stearothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

In accordance with the embodiments provided herein, various other PCR reagents can include an amplification reagent, which may include at least one primer or at least one pair of primers for amplification of a nucleic acid target, at least one probe and/or dye to enable detection of amplification, a ligase, a detergent (e.g., non-ionic detergents), nucleotides (dNTPs and/or NTPs), divalent magnesium ions, or any combination thereof, among others that would be recognized by one of ordinary skill in the art based on the present disclosure. In some embodiments, an amplification reagent and/or a nucleic acid target each may be present at an effective amount, such as an amount sufficient to enable amplification of a desired nucleic acid target in the presence of other necessary reagents.

In accordance with the embodiments provided herein, analysis reagents can include one or more primers, or any nucleic acid capable of, and/or used for, priming replication of a nucleic acid template. A primer may be DNA, RNA, an analog thereof (e.g., an artificial nucleic acid), or any combination thereof. A primer may have any suitable length, such as at least about 10, 15, 20, or 30 nucleotides. Exemplary primers are synthesized chemically. Primers may be supplied as at least one pair of primers for amplification of at least one nucleic acid target. A pair of primers may be a sense primer and an antisense primer that collectively define the opposing ends (and thus the length) of a resulting amplicon. In some embodiments, a primer is labelled for detection of the resulting amplicon. Suitable labels include fluorescent labels that are detected by known methods of fluorescence detection.

In accordance with the embodiments provided herein, analysis reagents can also include one or more probes, or any nucleic acid connected to at least one label, such as at least one dye. A probe may be a sequence-specific binding partner for a nucleic acid target and/or amplicon. The probe may be designed to enable detection of target amplification based on fluorescence or fluoresce resonance energy transfer (FRET). Methods herein may include a 5′ nuclease assay, such as with a TAQMAN probe. Analysis regents may include one or more labels or reporter molecules. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

In some embodiments, one or more analysis reagents are combined to form a composition or a kit, In some embodiments, the reagents necessary for amplification are combined into an concentrated or dried analysis reagent. In some embodiments, a composition can include any suitable PCR reagents that are required for carrying out an amplification reaction. For example, analysis reagents can include one or more PCR reagents, such as one or more of a primer or pair of primers for amplification of a nucleic acid target, a probe and/or dye to enable detection of amplification, a ligase, a polymerase, nucleotides (dNTPs and/or NTPs), divalent magnesium ions, or any combination thereof, among other reagents that would be recognized by one of ordinary skill in the art based on the present disclosure. In accordance with the embodiments provided herein, concentrations of the PCR reagents described above can vary, depending on specific reaction conditions and reagents used, as well as the desired DNA target to be amplified. One of skill in the art would readily recognize that any specific concentrations or concentration ranges provided herein for any PCR reagents will vary depending on the specific reaction conditions and reagents used and are not meant to be limiting.

In some embodiments, the present invention provides systems, kits, and methods for research, screening, and diagnostic applications. For example, in some embodiments, diagnostic applications provide detection and/or quantification of nucleic acids from athogenic entities (e.g., virus, bacteria, etc.) in a biological sample (e.g., from a subject). In some embodiments, the level, presence or absence of a pathogen, is used to provide a diagnosis or prognosis. In some embodiments, subjects are tested. Exemplary diagnostic methods are described herein. In some embodiments, nucleic acids from pathogens are detected in a sample from a subject In some embodiments, nucleic acids from pathogens are identified using the methods and reagents described herein.

Some embodiments herein unitize nucleic acid sequencing to detect/quantify target nucleic acid (e.g., from a pathogen) in a sample from a subject. The term “sequencing,” as used herein, refers to a method by which the identity of at least 10 consecutive nucleotides (e.g., the identity of at least 20, at least 50, at least 100, or at least 200 or more consecutive nucleotides) of a polynucleotide are obtained. The term “next-generation sequencing” refers to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies. In some embodiments, nucleic acids are amplified using primers that are compatible with use in, e.g., Illumina's reversible terminator method, Roche's pyrosequencing method (454), Life Technologies's sequencing by ligation (the SOLID platform) or Life Technologies's Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513: 19-39) and Morozova et al (Genomics. 2008 92:255-64), which are incorporated by reference for the general descriptions of the methods and the particular steps of the methods, including all starting products, reagents, and final products for each of the steps. In another embodiment, target nucleic acids may be sequenced using nanopore sequencing (e.g., as described in Soni et al. Clin Chem 2007 53: 1996-2001, or as described by Oxford Nanopore Technologies). Nanopore sequencing technology is disclosed in U.S. Pat. Nos. 5,795,782, 6,015,714, 6,627,067, 7,238,485 and 7,258,838 and U.S. Pat Appln Nos. 2006003171 and 20090029477. The isolated target nucleic acids may be sequenced directly or, in some embodiments, the target nucleic acids may be amplified (e.g., by PCR) to produce amplification products that sequenced. In certain embodiments, amplification products may contain sequences that are compatible with use in, e.g., Illumina's reversible terminator method, Roche's pyrosequencing method (454), Life Technologies's sequencing by ligation (the SOLID platform) or Life Technologies's Ion Torrent platform, as described above.

In some embodiments, provided herein is a method of detecting/analyzing a target nucleic acids in a biological sample for subsequent analysis, comprising: (a) combining the biological sample comprising cells with a dry or concentrated lysis reagent comprising proteinase K, SDS, and salt; and resuspending the dry or concentrated lysis reagent in the biological sample to generate a lysate; (b) combining the lysate with a dry or concentrated capture reagent and resuspending the dry or concentrated capture reagent in the lysate, wherein the capture reagent comprises a nucleic acid probe tethered to a capture moiety, wherein the capture moiety is biotin, and wherein the nucleic acid probe comprises a hybridization sequence that is complementary to a target sequence within the nucleic acids of the biological sample; (c) incubating the nucleic acid probe and the nucleic acids of the lysate at a temperature of 63-73° C. to generate a probe-bound nucleic acid solution; (d) combining the probe-bound nucleic acid with dry or concentrated capture-agent-coated magnetic beads and resuspending the dry or concentrated capture-agent-coated magnetic beads in the probe-bound nucleic acid at 70-80° C., wherein the capture agent is streptavidin; (e) incubating the probe-bound nucleic acid and capture-agent-coated magnetic beads at 63-73° C. and allowing the capture agent to bind to the capture moiety to generate a bead-captured nucleic acid suspension; (f) isolating bead-captured nucleic acids within the bead-captured nucleic acid suspension by exposing a portion of the bead-captured nucleic acid suspension to a magnetic field, and (A) holding the magnetic field in place while removing the liquid portion from the bead-captured nucleic acid suspension, or (B) moving the magnetic field to drag the bead-captured nucleic acids from the liquid portion; and (g) separating the isolated, bead-captured nucleic acids from a liquid portion of the bead-captured nucleic acid suspension; (h) combining the isolated bead-captured nucleic acid with a wash buffer; (i) resuspending the bead-captured nucleic acid in the wash buffer; (j) isolating the bead-captured nucleic acids within the wash buffer by exposing a portion of the bead-captured nucleic acids to a magnetic field; and (k) separating the isolated, bead-captured nucleic acids from the wash buffer; (l) combining the isolated bead-captured nucleic acid with a resuspension buffer; (m) resuspending the bead-captured nucleic acid in the resuspension buffer to generate a bead-captured nucleic acid resuspension; (n) combining the bead-captured nucleic acid resuspension with dry or concentrated analysis reagents and resuspending the dry or concentrated analysis reagents in the bead-captured nucleic acid resuspension, wherein the analysis reagents comprise primers and detectable labels for amplifying and detecting the target nucleic acids; and amplifying and detecting the target nucleic acid hybridized to the bead-bound capture reagent; wherein the method does not comprise a centrifugation step, a filtration step, or nucleic acid precipitation step; wherein the nucleic acids are not isolated from contaminants within the biological sample in steps (a) through (e); and wherein the wash buffer and resuspension buffer contain the same components.

In some embodiments, provided herein are methods of preparing a target nucleic acids in a biological sample for subsequent analysis, comprising: (a) combining the biological sample with a dry or concentrated reagent comprising lysis/digestion components and capture components, and resuspending the dry or concentrated reagent in the biological sample, wherein the lysis/digestion components comprise proteinase K, SDS, and salt, wherein the capture components comprise comprises a nucleic acid probe tethered to a capture moiety, wherein the capture moiety is biotin, and wherein the nucleic acid probe comprises a hybridization sequence that is complementary to a target sequence within the nucleic acids of the biological sample; (b) incubating biological sample at a temperature 90-100° C. to allow lysis of cell membranes and digestion of proteins within the sample to generate a lysate; (c) incubating the lysate at a temperature of 63-73° C. to allow binding of the hybridization sequence of the nucleic acid probe to the target sequence of the nucleic acid of the biological sample to generate a probe-bound nucleic acid solution; (d) combining the probe-bound nucleic acid with dry or concentrated capture-agent-coated magnetic beads and resuspending the dry or concentrated capture-agent-coated magnetic beads in the probe-bound nucleic acid at 70-80° C., wherein the capture agent is streptavidin; (e) incubating the probe-bound nucleic acid and capture-agent-coated magnetic beads at 63-73° C. and allowing the capture agent to bind to the capture moiety to generate a bead-captured nucleic acid suspension; (f) isolating bead-captured nucleic acids within the bead-captured nucleic acid suspension by exposing a portion of the bead-captured nucleic acid suspension to a magnetic field, and (A) holding the magnetic field in place while removing the liquid portion from the bead-captured nucleic acid suspension, or (B) moving the magnetic field to drag the bead-captured nucleic acids from the liquid portion; and (g) separating the isolated, bead-captured nucleic acids from a liquid portion of the bead-captured nucleic acid suspension; (h) combining the isolated bead-captured nucleic acid with a wash buffer; (i) resuspending the bead-captured nucleic acid in the wash buffer; (j) isolating the bead-captured nucleic acids within the wash buffer by exposing a portion of the bead-captured nucleic acids to a magnetic field; (k) separating the isolated, bead-captured nucleic acids from the wash buffer; (l) combining the isolated bead-captured nucleic acid with a resuspension buffer; and (m) resuspending the bead-captured nucleic acid in the resuspension buffer to generate a bead-captured nucleic acid resuspension; (n) combining the bead-captured nucleic acid resuspension with dry or concentrated analysis reagents and resuspending the dry or concentrated analysis reagents in the bead-captured nucleic acid resuspension, wherein the analysis reagents comprise primers and detectable labels for amplifying and detecting the target nucleic acids; and (o) amplifying and detecting the target nucleic acid hybridized to the bead-bound capture reagent; wherein the method does not comprise a centrifugation step, a filtration step, or nucleic acid precipitation step; wherein the nucleic acids are not isolated from contaminants within the biological sample in steps (a) through (e); and wherein the wash buffer and resuspension buffer contain the same components.

In some embodiments, the methods herein are conducted using any suitable reaction vessels (e.g., tubes, wells, chambers within a device, etc.), manual laboratory instruments (e.g., hand pipettes, heating blocks, vortexer, etc.), or automated instruments (e.g., robotics, microfluidics, liquid handlers, self-contained cartridge, fluorimeter, etc.). In some embodiments, the method is performed manually (e.g., under the control of a human operator). In some embodiments, the movement, combining, and or mixing of liquids and regents is conducted by manual pipetting. In some embodiments, the performance of one or more (e.g., all) of the method steps is automated. In some embodiments, one or more (e.g., all) of the method steps are performed within a single-use cartridge. In some embodiments, the single-use cartridge contains all dry and liquid reagents and buffers for performing the method steps. In some embodiments, the single use cartridge interfaces with an instrument that comprises components for combining and mixing reagents, heating elements, magnet(s), and fluorescence detection. Suitable systems comprising a single-use cartridge and complementary instrument are described in U.S. Prov. App. 63/180,270; incorporated by reference in its entirety. In some embodiments, one or more (e.g., all) of the method steps are performed by an automation instrument (e.g., a high throughput robotic platform capable of transferring liquids between well, mixing solutions/suspensions, heating, moving magnetic fields, detecting fluorescence, etc.).

In some embodiments, provided herein are systems or kits comprising: (a) a lysis reagent (dry or concentrated) capable of digesting cell membranes and degrading cellular proteins; (b) a capture reagent (dry or concentrated) comprising a nucleic acid probe tethered to a capture moiety; (c) capture-agent-coated magnetic beads (dry or concentrated); (d) amplification/detection reagents (dry or concentrated); and (e) a wash/resuspension buffer solution.

In other embodiments, provided herein are systems or kits comprising: (a) a lysis/capture reagent (dry or concentrated) comprising (1) lysis/digestion components capable of digesting cell membranes and degrading proteins, and (2) a nucleic acid probe tethered to a capture moiety; (b) capture-agent-coated magnetic beads (dry or concentrated); (c) amplification/detection reagents (dry or concentrated); and (d) a wash/resuspension buffer solution.

In other embodiments, provided herein are systems or kits comprising: (a) a lysis reagent (dry or concentrated) comprising proteinase K, SDS, and one or more salts; (b) a capture reagent (dry or concentrated) comprising a nucleic acid probe tethered to a biotin capture moiety; (c) capture-agent-coated magnetic beads (dry or concentrated), wherein the capture agent is streptavidin; (d) amplification/detection reagents (dry or concentrated) comprising primers, detectable labels, and nucleotides; and (e) a buffer solution.

In other embodiments, provided herein are systems or kits comprising: (a) a lysis/capture reagent (dry or concentrated) comprising (1) lysis/digestion components comprising proteinase K, SDS, and one or more salts, and (2) a nucleic acid probe tethered to a capture moiety; (b) capture-agent-coated magnetic beads (dry or concentrated), wherein the capture agent is streptavidin; (c) amplification/detection reagents (dry or concentrated) comprising primers, detectable labels, and nucleotides; and (d) a buffer solution.

In some embodiments, systems and kits further comprise a biological sample comprising nucleic acid (e.g., within a virus or cell (e.g., bacteria, protozoa, etc.).

In some embodiments, systems and kits further comprise disposable laboratory products for manually using the system or kit for the capture, isolation, amplification, and detection of a target nucleic acid from a biological sample comprising cells. In some embodiments, disposable laboratory products comprise pipette tips, reaction tubes, and or a microwell plate.

In some embodiments, systems and kits further comprise a single-use cartridge containing the components of the systems/kits herein, wherein the single-use cartridge is capable of interfacing with an instrument that comprises components for combining and mixing reagents, heating elements, and a magnet.

In an exemplary protocol, for an exemplary cartridge device (e.g., the device pictured in FIGS. 1-9), a device is provided containing buffer (e.g., 800 μl) in chamber two (50) and a sample (e.g., liquid sample (e.g., about 1,000 μl) in chamber one (46). The device 10 is inserted into a complementary instrument comprising a pressure source for interfacing with the transfer capsule 26 of the device 10. The pressure source retrieves the transfer capsule 26 from the cavity 42, and via the second access port 74, draws buffer (e.g., about 350 μl) from chamber two (50) and transfers the buffer the buffer to chamber six (152) via the access port 172 for that chamber. A portion of the transferred buffer flows via the microfluidics to the reaction chamber 304.

The pressure source and transfer capsule 26 then withdraw the sample from chamber one (46), via the access port 66 for that chamber and transfer it to chamber three (140) via the access port 160 for that chamber. As the sample flows from the third access port 160 through the cross-channel portion 204, a lyophilized reagent 208 positioned in the cross-channel portion 204 is rehydrated when liquid flows through the third channel 184. The lyophilized reagent 208 contains reagents for cell lysis and digestion of cellular components (e.g., SDS, proteinase K, etc.), as well as nucleic acid capture probes (e.g., sequence-specific probes comprising a handle (e.g., biotin) that allows for capture of nucleic acids hybridized to the probes). The orientation of chamber three (140) within the instrument allows heating of the contents of chamber three (140), for example to about 95° C. to facilitate cell lysis. The sample is lysed and digested in chamber three (140). The pressure source and transfer capsule 26 are used to mix the sample and reagents by drawing the sample/reagents into an out of the transfer capsule via the third access port 160 and third channel 184 and by injecting air bubbles into the sample/reagents.

The pressure source and transfer capsule 26 then withdraw the sample/reagents from chamber three (140), via the access port 160 for that chamber and transfer it to chamber four (144) via the access port 164 for that chamber. The sample/reagents cool in chamber four (144) allowing the capture probe to hybridize with complementary nucleic acids in the sample.

The pressure source and transfer capsule 26 then withdraw the sample/reagents from chamber four (144), via the access port 164 for that chamber and transfer it to chamber five (148) via the access port 168 for that chamber. As the sample/reagents enter chamber five (148), paramagnetic particles (PMPs) comprising a binding moiety (e.g., streptavidin) for binding the handle on the capture probes are resolubilized by the sample/reagents. Resolubilization is assisted by withdrawing and ejecting fluid from the transfer capsule into and out of chamber five (148).

The pressure source and transfer capsule 26 then withdraw the sample/reagents from chamber five (148), via the access port for that chamber and transfer it to chamber four (144) via the access port 168 for that chamber. The PMPs are allowed to bind to the capture probes, thereby capturing the associated nucleic acids.

The pressure source and transfer capsule 26 then withdraw the sample/reagents from chamber four (144), via the access port 164 for that chamber and transfer it to chamber five (148) via the access port 168 for that chamber. When transferring chamber four to chamber five, the instrument magnet is held at the tip of the transfer capsule, so that the PMPs are collected during dispensing and aspiration. The instrument magnet is then placed adjacent to the bottom of chamber five, thereby forming a pellet of the PMPs, with the capture probes and bound nucleic acids attached. The liquid from chamber five (148) is removed by the pressure source and transfer capsule 26 via the access port 168 for that chamber and deposited into chamber three (140) via the access port 160 for that chamber.

The pressure source and transfer capsule 26 then withdraw the buffer from chamber two (50), via the access port 74 for that chamber and transfer it to chamber five (148) via the access port 168 for that chamber. The PMPs in chamber five (148) are resuspended in the buffer via mixing with the pressure source and transfer capsule 26. The instrument magnet is then placed adjacent to the bottom of chamber five, thereby forming a pellet of the PMPs, with the capture probes and bound nucleic acids attached. The liquid from chamber five (148) is removed by the pressure source and transfer capsule 26 via the access port 168 for that chamber and deposited into chamber three (140) via the access port 160 for that chamber.

The pressure source and transfer capsule 26 then withdraw the buffer from chamber six (152), via the access port 172 for that chamber and transfer it to chamber five (148) via the access port 168 for that chamber. The PMPs in chamber five (148) are resuspended in the buffer via mixing with the pressure source and transfer capsule 26.

The pressure source and transfer capsule 26 then withdraw the buffer from chamber five (148) via the access port 168 and transfer it to chamber six (152), via the access port 172 for that chamber. The instrument magnet is positioned adjacent to the bottom of chamber six (152) as the liquid is added to the chamber in order to pre-collect the PMPs at the bottom of the chamber.

The instrument magnet is then used to pellet the PMPs and transfer the PMPs via the inlet channel 312 to the reaction chamber 304. The wax seals of the vent channel (308) and inlet channel (312) are melted using instrument heaters and allowed to solidify in the vent of the reaction chamber and inlet channel. PCR is then performed on the PMP-bound nucleic acids, using fluorescently-labelled primers, and the amplified nucleic acids are detected using instrument-based fluorescence detection.

In some embodiments, a cartridge device comprising the two storage chambers (C1 and C2), four processing chambers (C3, C4, C5, and C6), and one reaction chamber is provided. In some embodiments, C1 is a sample chamber. An environmental, biological, or research sample (e.g., comprising cells and/or a target analyte) is placed into C1 and the chamber is sealed (e.g., capped). In some embodiments, C2 is a buffer storage chamber. The buffer that will be used for sample processing and analyte detection is contained in C2. In some embodiments, a single buffer is used. In embodiments in which multiple buffers are required, a device may comprise multiple buffer storage chambers (e.g., C2A, C2B, etc.). In some embodiments, the storage chambers are sized to contain a sufficient volume of sample and buffer to perform the various processing and detection steps (e.g., the storage chambers are of greater width than the processing chambers). In some embodiments, C3 is a sample digestion and/or cell lysis chamber. Upon addition of the sample to C3, a reagent pellet within the access channel to C3 is dissolved, exposing the sample to the reagents necessary for sample processing and/or analyte detection (e.g., lysis reagent, digestion reagent, capture probe, etc.). In some embodiments, C3 is positioned on the cartridge to align with one or more heaters of the complementary instrument. In some embodiments, the sample in C3 is exposed to an appropriate temperature to facilitate the appropriate steps of sample processing for the specific sample type and assay protocol. In some embodiments, C4 is an analyte binding/hybridization chamber. In some embodiments, C4 is positioned on the cartridge to align with one or more heaters of the complementary instrument. In some embodiments, C4 is maintained at a temperature (e.g., lower than that of C3) to allow for capture reagents to bind/hybridize to the target analyte. In some embodiments, the width of C3 and C4 is suitable to accommodate close alignment of the chambers with the heater(s) (e.g., C3 and C4 are narrower than the storage chambers). In some embodiments, C5 is a capture chamber. In some embodiments, upon addition of the sample to C5, a pellet comprising PMPs within the access channel to C5 is dissolved, exposing the analyte (e.g., bound to capture probes) to the PMPs capable of binding to the capture probes. In some embodiments, C5 is positioned on the cartridge to align with a magnetic transfer element of the instrument. The magnetic transfer element allows the PMPs in C5 (e.g., bound to analyte-bound capture probes) to be pelleted, moved, and otherwise physically manipulated (e.g., smeared). In some embodiments, the width of C5 is suitable to accommodate close alignment of the chamber with the magnetic transfer element of the complementary instrument. In some embodiments, C6 is a transfer chamber. In some embodiments, pelleted PMPs in C6 can be transferred using the magnetic transfer element of the complementary instrument, via a transfer channel, into the reaction chamber. In some embodiments, the width of C6 is suitable to accommodate close alignment of the chamber with the magnetic transfer element of the complementary instrument. In some embodiments, upon addition of the pellet/sample to the reaction chamber, a detection reagent pellet is dissolved, exposing the sample to the reagents necessary for analyte detection (e.g., primers, probes, antibodies, etc.). In some embodiments, the reaction chamber is sized and configured to allow close alignment of the chamber with heaters, fluorimeter, and/or other components of the complementary instrument to allow analyte detection.

Alternative protocols for the exemplary devices, systems, and components thereof, as well as alternative devices, systems, and components with different layouts are within the scope of the embodiment herein.

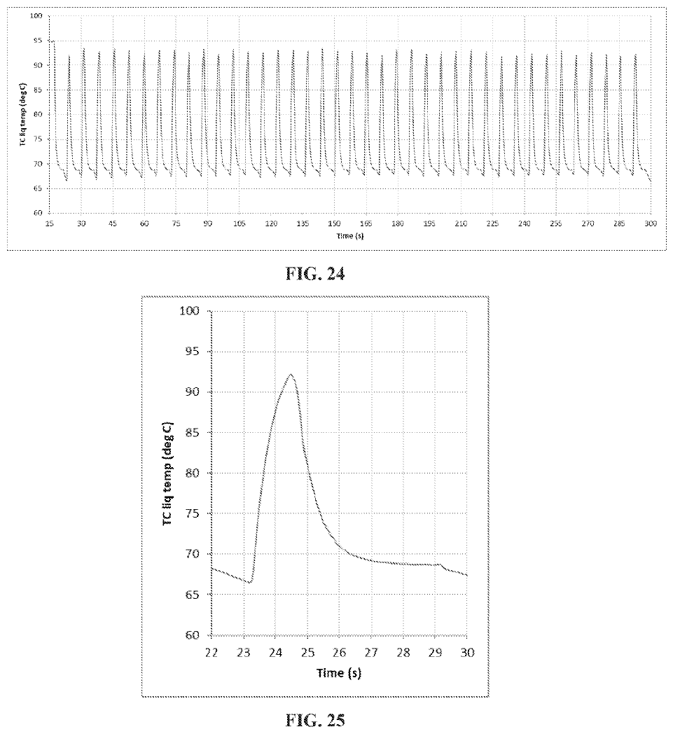

With reference to FIGS. 24 and 25, PCR can be performed by cycling the temperature of the reaction chamber 66 between two temperatures. For example, when the first and second heat transfer devices 22A, 22B at the first temperature (the high temperature pair) come into contact with a PCR chamber at a lower temperature, thermal energy flows from the heat transfer devices 22A, 22B into the PCR chamber. In some embodiments, the rate of thermal energy flow is proportional to the temperature difference, and as the PCR chamber heats up and the difference in temperature approaches zero, the flow of thermal energy stops. Conversely, when the third and fourth heat transfer devices 22C, 22D at the second temperature (the low temperature pair) come into contact with the PCR chamber at a higher temperature, thermal energy flows out of the PCR chamber and into the heat transfer devices 22C, 22D. FIG. 24 is a graph of the liquid temperature in a PCR chamber as a function of time as it cycles between the low temperature heat transfer devices (e.g., 22C, 22D) and the high temperature heat transfer devices (e.g., 22A, 22B). In the illustrated embodiment, approximately 40 thermal cycles are achieved within a 300 second time frame. FIG. 25 is an enlarged portion of the graph of FIG. 24 and illustrates one of the plurality of thermal cycles.

With reference to FIG. 26, the microfluid section 62 of the cartridge 50 includes a first wax seal 182 and a second wax seal 186. The first wax seal 182 is positioned adjacent the microfluid vent channel 70 and the second wax seal 186 is positioned adjacent the microfluid inlet channel 74. To maintain stable concentrations of reagents and amplicon, and prevent contamination of the instrument, PCR is conducted in a closed system (e.g., the reaction chamber 66 is sealed). The wax seals 182, 186 are configured to seal the reaction chamber 66 from two ends (i.e., the inlet end and the vent end). The open-air microfluid vent channel 70 leading from the reaction chamber 66 is utilized for initial buffer fluid priming of the dried reagents and air purge. After priming, paramagnetic particles carrying the target are transferred into the reaction chamber 66 through the microfluid inlet channel 74. Once the target is in the reaction chamber 66, the microfluid vent channel 70 is closed (i.e., sealed) by melting the first wax seal 182 (by, for example, a heater, heat transfer device, etc.). The molten wax fills the empty space and nearby microfluid vent channel 70 and then hardens, sealing the microfluid vent channel 70. After the first wax seal 182 is melted, the second wax seal 186 is then melted in a similar fashion, sealing off the microfluid inlet channel 74 and closing the reaction chamber 66. Therefore, in some embodiments, the first wax seal 182 in the microfluid vent channel 70 is melted and hardened before the second wax seal 186 in the microfluid inlet channel 74. Doing so ensures any air entrapped around the second wax seal 186 builds pressure to prevent molten wax from the second wax seal 186 from flowing into the reaction chamber 66. Wax in the reaction chamber 66 would affect the fluorescent optical readings.

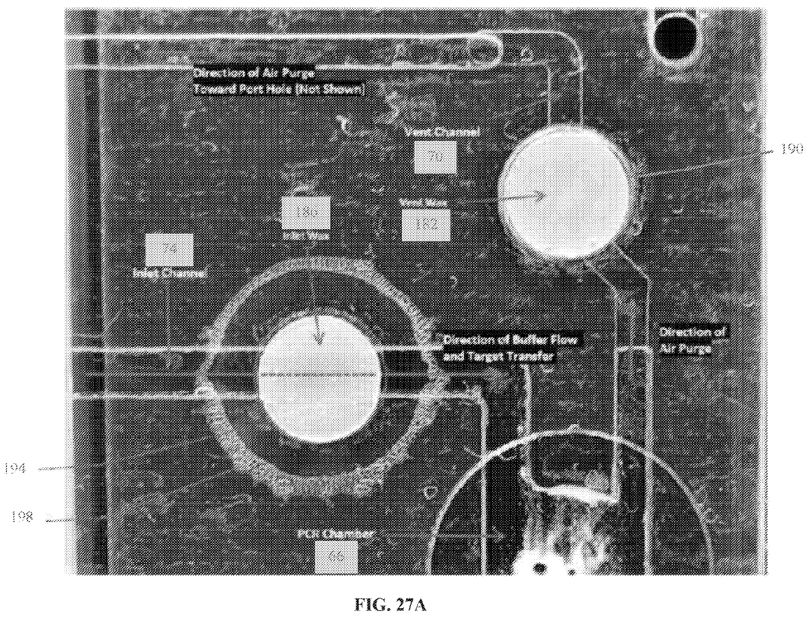

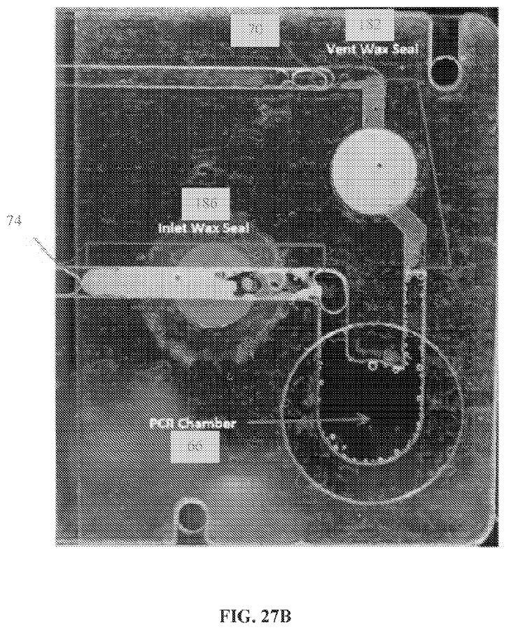

With reference to FIGS. 27A and 27B, the first and second wax seals 182, 186 are initially in a first solid state with the corresponding channels 70, 74 open (FIG. 27A) and are modifiable to a second solid state with the corresponding channels 70, 74 sealed (i.e., closed) (FIG. 27B). The wax seals 182, 186 enter a molten state between the first solid state and the second solid state. In the illustrated embodiment, the first and second wax seals 182, 186 are cylindrically shaped in the initial, first solid state (FIG. 27A) and do not occlude (obstruct or seal) their corresponding channels 70, 74. In response to the application of heat, the first and second wax seals 182, 186 melt and flow into the channels 70, 74. After the application of heat is removed, the molten wax rehardens and enters the second solid state (FIG. 27B) and forms a solid wax seal positioned within the channels 70, 74.

The first wax seal 348 in the first solid state is initially positioned above the microfluid vent channel 308 within a cutout 190 (FIG. 27A). In the initial solid state of the first wax seal 182, air can easily pass around and beneath the wax seal 182. When the wax seal 182 is melted, molten wax fills the empty surrounding space (FIG. 27B). In some embodiments, the volume of first wax seal 182 is less than the volume of the second wax seal 186.

The second wax seal 186 is positioned adjacent the microfluid inlet channel 74 and extends beyond the laminate layers, creating a tented air pocket 194 (FIG. 27A) around the perimeter of the second wax seal 186. In other words, the second wax seal 186 causes the cartridge cover to deform during assembly resulting in the tented air pocket 194. Despite the second wax seal 186 being positioned above the microfluid inlet channel 74, fluid and target transfer through the microfluid inlet channel 74 remains possible by utilizing hydrostatic head at the onset of the microfluid inlet channel and the amount of detergent in the fluid.

In some embodiments, the microfluid inlet channel 74 is taller than the microfluid vent channel 70. The microfluid vent channel 70 has a cross-sectional area of approximately 0.051 mm² (e.g., 0.051 mm tall×1 mm wide). Likewise, the microfluid inlet channel 74 has a cross-sectional area of approximately 0.54 mm² (e.g., 0.36 mm tall×1.5 mm wide). The microfluid vent channel 70 has a smaller cross-sectional area than the microfluid inlet channel 74 because the microfluid vent channel 70 directs only airflow whereas the microfluid inlet channel 74 must allow passage of liquid buffer and solid particles containing genetic targets. Therefore, the amount of wax required in the wax seal 186 for the microfluid inlet channel 74 is greater than the amount of wax in the wax seal 182 for the microfluid vent channel 70.

In some embodiments, the tented air pocket 194 is approximately 0.38 mm taller than the surrounding laminate. When the wax seal 186 is melted by clamping heaters, the tented air pocket 194 is depressed. Air remaining entrapped within hardened wax seals creates potential for fluid leakage. As such, it is important the air has a route to exit the system during the wax melting process to not compromise seal integrity. In the illustrate embodiment, spacing of at least approximately 2 mm is provided between any laser cut feature and an edge of the laminate to provide sufficient surface area for a strong adhesive bond to form. In other words, narrow adhesive contact areas are vulnerable to leakage of leakage and failure. Specifically, the perimeter 198 of the tented air pocket 194 is positioned at least approximately 2 mm away from the reaction chamber 66. In the illustrated embodiment, there is at least approximately 2 mm of spacing from the tented air pocket 194 and any exposed laminate edge.

The wax seals 182, 186 provide several advantages. The hardened wax seals 182, 186 are advantageously configured to withstand the pressures in the reaction chamber 66 experienced during thermal cycling, which involves alternated clamping of the reaction chamber with heater temperatures in the range of approximately 50° C. and approximately 95° C. In other words, the combination of high temperatures and fluid displacement from mechanical clamping puts stress on the wax seals 182, 186 that are withstood. Although the heaters may not directly contact wax seals during thermal cycling, wax with a high melting temperature (e.g., paraffin wax with a melting temperature of at least approximately 85° C.) is selected in some embodiments to ensure the wax seals are not inadvertently re-melted by heaters associated with the reaction chamber. In some embodiments, the wax seals go through more than one cycle of melting and hardening (i.e., greater than one thermal cycle).

In the illustrated embodiment, the wax seals 182, 186 are initially cylindrically shaped (i.e., coin-shaped) (e.g., approximately 4.5 mm in diameter by approximately 0.43 mm thick). The rotational symmetry of a circular geometer of a wax seal reduces the risk of misplacement during manufacturing of the cartridge 50. Furthermore, a design with identical wax seals simplifies production. In other embodiments, the wax seals are initially elliptical-shaped.

The wax seals 182, 186 can be melted within a range of approximately 86° C. (i.e., the wax melting point) and approximately 95° C. (i.e., the default temperature setting of a PCR heater). The melting duration, or the amount of time that the PCR heaters are clamped onto a wax seal, can be modulated in tandem with the melting temperature to ensure a good seal. For example, if a wax seal is melted at too high of a temperature for too long, molten wax will diffuse further away from the sealing site—reducing the material density and mechanical integrity of the seal. In some embodiments, the melting procedure melts the wax seals 182, 186 by applying a hotter heater for a duration within a range of approximately 4 to approximately 5 seconds. Then, the wax seals 182, 186 are clamped with a cooler heater with a setpoint below the wax melting temperature for a duration of approximately 1.5 second. To reduce overall processing time, the wax seals 182, 186 are melted while the hot PCR heaters are cooling from approximately 95° C. to approximately 86° C. (rather than at a fixed temperature).

In some embodiments, the density of the wax seals 182, 186 is approximately 0.9 g/mL, which is slightly less dense than the fluids surrounding them at 1 g/mL. In the illustrated embodiment, gravity acts downwards during the melting and subsequent hardening of the wax seals. As such, the orientation of gravity can influence the movement of the molten wax. For example, molten wax may flow upwards when gravity is acting downwards. The direction of the molten wax flow is also affected by the surface area and position of the heater utilized to melt the wax seals. For example, when clamping with a heater, if it is not concentric but rather offset in one direction, the molten wax will tend to flow in the offset direction. Likewise, the heater clamping force is modulated as a function of the relative positions of the front and back sides of the heater, which can be mounted on low spring-constant springs, for example. In some embodiments, the plastic laminate layers are less rigid and capable of deforming in response to the heater clamping force, extruding and pushing the wax seals beyond the boundary of the heater surface area.

In some embodiments, the molten wax seals are cooled by clamping the molten wax seals with a cooler heater (i.e., a heater with a temperature less than the wax melting temperature). In other embodiments, the molten wax seals are cooled hardening in ambient air. Clamping the molten wax seals with the cooler heaters to cool the wax cause the wax to harden more quickly. Time to cool in ambient air is approximately 6 to approximately 8 second, whereas time to cool by clamping is approximately 2 seconds.

In some embodiments, the bonding of the wax seals 182, 186 is improved by exposing the paraffin wax to a layer of acrylic-based adhesive tape, instead of other plastic films such as polyester or polycarbonate. Improved bond quality between melted wax and a channel wall can decrease the likelihood of fluid leakage through the hardened seal.

In some embodiments, the devices, systems, components, reagents, and methods described herein find use in the amplification and/or detection of nucleic acids in a sample. In some embodiments, such devices, systems, components, reagents, and methods find use with single-use assay cartridges, multi-use assay cartridges, cartridge/instrument combinations, high throughput multiplex instruments, robotics, separate sample preparation and amplification/detection components, combined sample preparation and amplification/detection components, etc. In some embodiments, methods are provided herein for the analysis of nucleic acid amplification reactions performed using the devices, systems, components, reagents, and methods described herein, and/or performed with other devices, systems, components, reagents, and methods understood in the field.

Certain embodiments herein utilize real-time PCR or quantitative PCR (qPCR) to amplify and detect a target nucleic acid. In conventional PCR, the amplified DNA product, or amplicon, is detected in an end-point analysis. In real-time PCR, the accumulation of amplification product is measured as the reaction progresses, in real time, with product quantification after each cycle. By analyzing the accumulation of product after each cycle, the amount of target nucleic acid in the original sample can be quantitated. In some embodiments, real-time detection of PCR products is achieved by the inclusion of a reporter molecule in the PCR reaction well that yields increased signal with an increasing amount of product DNA (e.g., signal from the reporter is proportional to the amount of amplicon produced). In particular embodiments, the reporter is a fluorescent reporter molecule (fluorophore) and the signal detected after each completed PCR cycle is a fluorescent signal. Various fluorescence chemistries can be employed as reporters, including DNA-binding dyes, fluorescently-labeled target sequence specific probes, and/or fluorescently-labelled primers.

Real-time PCR allows determination of the initial number of copies of template nucleic acid (target sequence) with accuracy and high sensitivity over a wide dynamic range. Real-time PCR results can either be qualitative (the presence or absence of a sequence) or quantitative (copy number). In some embodiments, the amount of an organism or pathogen (e.g., viral load) is determined based on the amount of target nucleic acid detected in a sample.

Amplification during qPCR occurs in two phases, an initial exponential phase followed by a non-exponential plateau phase. During the exponential phase, the amount of PCR product approximately doubles in each cycle. As the reaction proceeds, however, reaction components are consumed, and ultimately one or more of the components becomes limiting. At this point, the reaction slows and enters the non-exponential plateau phase. During the initial part of the exponential phase, fluorescence remains at background levels, and increases in fluorescence are not readily detectable, even though the reaction product is accumulating exponentially. Once enough amplified product accumulates, detectable fluorescence signal will be detectable through the remainder of the reaction. The cycle at which fluorescence from amplification product exceeds the background fluorescence has been referred to as threshold cycle (Ct) or the quantification cycle (Cq). Because the Cq value is measured in the exponential phase when reagents are not limited, real-time qPCR can be used to reliably and accurately calculate the initial amount of template present in the reaction based on the known exponential function describing the reaction progress. The Cq of a reaction is determined mainly by the amount of template present at the start of the amplification reaction. If a large amount of template is present at the start of the reaction, relatively few amplification cycles will be required to accumulate enough product to give a fluorescence signal above background. Thus, the reaction will have a low, or early, Cq. In contrast, if a small amount of template is present at the start of the reaction, more amplification cycles will be required for the fluorescence signal to rise above background. Thus, the reaction will have a high, or late, Cq. This relationship forms the basis for the quantitative aspect of real-time PCR.

In some embodiments, provided herein are methods of performing qPCR, analyzing the data to determine Cq, and determining the copy number of the target sequence based thereon. In some embodiments, methods are provided for Cq determination from qPCR results that are independent of the absolute levels of the fluorescence readings. Such methods eliminate the need to calibrate each instrument, and allows for signal intensities to change as optical components (LEDs, interference filters) age. In some embodiments, the methods herein allow comparison of Cq values determined on separate instruments (or the same instrument at different points in time), without calibration between instruments or timepoints.

In some embodiments, the essential steps of the method for performing and analyzing qPCR are obtaining fluorescence readings after each cycle of a qPCR protocol (e.g., 40 cycles); reduce variability in fluorescence readings by calculating a moving average for each cycle; identifying the cycle with maximum increase in fluorescence (normDelta); if maximum normDelta is greater than a cutoff value, then the moving average signals from the preceding cycles are reviewed to identify the earliest cycle in which normDelta exceeds a second cutoff value (e.g., lower cutoff); fitting a straight line to the a plurality (e.g., 3, 4, 5, 6, 7, 8, etc.) of signals (moving average signals) preceding (e.g., sequential signals, signals immediately preceding) the earliest cycle in which normDelta exceeds the second cutoff value (e.g., lower cutoff); fitting a quadratic curve to a plurality (e.g., 3, 4, 5, 6, 7, 8, etc.) of signals (moving average signals) following (e.g., sequential signals, signals immediately following) the earliest cycle in which normDelta exceeds the second cutoff value (e.g., lower cutoff); and determining Cq as the calculated cycle where the difference between the fitted line and the fitted curve differ by a specified value.

In some embodiments, methods comprise: (a) performing a multicycle amplification reaction on a sample suspected of containing a target nucleic acid in the presence of a detectable reporter to produce an amplification product; (b) detecting a signal from the detectable reporter that correlates with the amount of detectable reporter incorporated into the amplification product after each cycle of the amplification reaction; (c) identifying earliest cycle with an increase in signal that is greater than a cutoff value; (d) fit a linear equation to a plurality of signals from cycles earlier than the earliest cycle with an increase in signal that is greater than the threshold value; (e) fit a quadratic curve to a plurality of signals from cycles later than the earliest cycle with an increase in signal that is greater than the threshold value; (f) identify the cycle for which the difference in signal for the linear equation and the quadratic curve is equal to a threshold value (Cq); wherein Cq is inversely proportional to the amount of target nucleic acid present in the sample.

In some embodiments, methods comprise: (a) performing a multicycle amplification reaction on a sample suspected of containing a target nucleic acid in the presence of a detectable reporter to produce an amplification product; (b) detecting a signal from the detectable reporter that correlates with the amount of detectable reporter incorporated into the amplification product after each cycle of the amplification reaction; (c) identifying the cycle with the maximum increase in signal; (d) if the maximum increase in signal is greater than a cutoff value, then determine the earliest cycle prior to the cycle with the maximum increase in signal that has an increase in signal that is greater than a second cutoff value (e.g., lower cutoff); (e) fit a linear equation to a plurality of signals from cycles earlier than the earliest cycle with an increase in signal that is greater than the second cutoff value (e.g., lower cutoff); (g) fit a quadratic curve to a plurality of signals from cycles later than the earliest cycle with an increase in signal that is greater than the second cutoff value (e.g., lower cutoff); (h) identify the cycle for which the difference in signal for the linear equation and the quadratic curve is equal to a threshold value (Cq); wherein Cq is inversely proportional to the amount of target nucleic acid present in the sample.

In some embodiments, methods comprise: (a) performing a multicycle amplification reaction on a sample suspected of containing a target nucleic acid in the presence of a detectable reporter to produce an amplification product; (b) detecting a signal from the detectable reporter that correlates with the amount of detectable reporter incorporated into the amplification product after each cycle of the amplification reaction; (c) calculating a moving average signal of the detected signal for each cycle of the amplification reaction; (d) identifying earliest cycle with an increase in moving average signal that is greater than a cutoff value; (e) fit a linear equation to a plurality of moving average signals from cycles earlier than the earliest cycle with an increase in moving average signal that is greater than the threshold value; (f) fit a quadratic curve to a plurality of moving average signals from cycles later than the earliest cycle with an increase in moving average signal that is greater than the threshold value; (g) identify the cycle for which the difference in moving average signal for the linear equation and the quadratic curve is equal to a threshold value (Cq); wherein Cq is inversely proportional to the amount of target nucleic acid present in the sample.

In some embodiments, methods comprise: (a) performing a multicycle amplification reaction on a sample suspected of containing a target nucleic acid in the presence of a detectable reporter to produce an amplification product; (b) detecting a signal from the detectable reporter that correlates with the amount of detectable reporter incorporated into the amplification product after each cycle of the amplification reaction; (c) calculating a moving average signal of the detected signal for each cycle of the amplification reaction; (d) identifying the cycle with the maximum increase in moving average signal; (e) if the maximum increase in moving average signal is greater than a cutoff value, then determining the earliest cycle prior to the cycle with the maximum increase in moving average signal that is also has an increase in moving average signal that is greater than a second cutoff value (e.g., lower cutoff); (f) identifying earliest cycle with an increase in moving average signal that is greater than a second cutoff value (e.g., lower cutoff); (g) fit a linear equation to a plurality of moving average signals from cycles earlier than the earliest cycle with an increase in moving average signal that is greater than the threshold value; (h) fit a quadratic curve to a plurality of moving average signals from cycles later than the earliest cycle with an increase in moving average signal that is greater than the threshold value; (i) identify the cycle for which the difference in moving average signal for the linear equation and the quadratic curve is equal to a threshold value (Cq); wherein Cq is inversely proportional to the amount of target nucleic acid present in the sample.

In some embodiments herein, Cq is a calculated cycle number where the fluorescence increases to a specified percent above the baseline. In particular embodiments, this calculation is made at the breakpoint cycle where fluorescence is determined to be significantly above the baseline.

Due to variability in the fluorescence readings (F[i]), in some embodiments, herein a moving average (MAF[i]) is calculated for each cycle (i) and used for subsequent analysis steps. In some embodiments, MAF[i] is calculated for each cycle, i, using the signal for the cycle, F[i], and the immediately preceding and following cycles (e.g., 1-3 immediately preceding and following cycles), for example, according to the following equation:

$$MAF [ i ] = ( F [ i - 2 ] + F [ i - 1 ] + F [ i ] + F [ i + 1 ] + F [ i + 2 ] ) / 5$$

In some embodiments, the rate of change of signal for each cycle is determined by calculating the difference between signal (moving average signal)×cycles (e.g., 1 cycle, 2 cycles, 3 cycles, 4 cycles, 5 cycles, 6 cycles, 7 cycles, 8 cycles, or more) before and after a given cycle, and dividing by the signal (moving average signal) for that cycle. For example, if the cycle number i, then the normalized difference (normDelta[i]), expressed as a percentage, when calculated using a range of signals 5 cycles prior and 5 cycles after i, can be determined:

$$normDelta [ i ] = 100 * MAF [ i + 5 ] [ i - 5 ] / MAF [ i ]$$

In some embodiments, if the largest normDelta between cycles 5 and 35 is less than cutoffHighNormDelta, then there is no breakpoint. In some embodiments, the value of the cutoff (cutoffHighNormDelta) depends on the cycle. In some embodiments, between cycles 5 and 24, cutoffHighNormDelta is 8; between cycles 25 and to 34, cutoffHighNormDelta is 4, and at cycle 35, cutoffHighNormDelta is 2.5. In some embodiments, a cutoff may range between 1.5 and 15 (e.g., 1.5, 2.0, 2.5, 3.0, 3.5, 4.0, 4.5, 5.0, 6.0, 7.0, 8.0, 9.0, 10, 11, 12, 13, 14, 15, or ranges therebetween). In some embodiments, different cutoffs may be applied to different ranges of cycles (e.g., 5-10, 11-15, 16-20, 21-25, 26-30, 31-35, or any other suitable ranges of cycles). If the maximum normDelta is greater than the cutoff, then a search begins looking at earlier cycles to find when it falls below cutoffLowNormDelta. In some embodiments, cutoffLowNormDelta is lower than cutoffHighNormDelta. In some embodiments, the value of the cutoff (cutoffLowNormDelta) depends on the cycle. In some embodiments, cutoffLowNormDelta is not based on the cycle. In some embodiments, a cutoff (cutoffLowNormDelta) may range between 1.5 and 6 (e.g., 1.5, 2.0, 2.5, 3.0, 3.5, 4.0, 4.5, 5.0, 6.0, or ranges therebetween). In some embodiments, the earliest cycle above cutoffLowNormDelta is the breakpoint cycle.

In some embodiments, if a breakpoint cycle is found, a linear equation is fit to the fluorescence signal (e.g., moving average) at the breakpoint cycle and several cycles before it (e.g., 2-6 previous cycles). If j=0 is the breakpoint cycle, then in certain embodiments the equation of the linear fit is:

$$FitL [ i ] = b 0 L + b1L \times C [ j ] , j = - 4 to 0$$

where C[j] is the cycle number and FitL[j] the linear estimate of fluorescence. b0L is the intercept of the line and b1L the slope.

In some embodiments, if a breakpoint cycle is found, a curve (e.g., quadratic curve) equation is fit to the fluorescence signal (e.g., moving average) at the breakpoint cycle and several cycles following it (e.g., 2-6 following cycles). If j=0 is the breakpoint cycle, then in certain embodiments the equation of the quadratic fit is:

$$FitR [ j ] = b 0 R + b1R \times C [ i ] + b1R \times C [ i ] \hat{} 2 , i = 0 to 4$$

where FitR[j] is the quadratic estimate of fluorescence with parameters b0R, b1R and b2R. The coefficients of FitL (b0L and b1L) are estimated by minimizing the sum of squared differences between FitL and MAF, as are the coefficients of FitR (B0R, b1R, b2R). Cq is the solution to the quadratic equation:

$$100 * \{ ( b 0 L + b 1 L \times + Cq ) - ( b 0 R + b 1 R \times Cq \hat{} 2 ) \} / b 0 L = calcCq$$

$$where calCq = 2$$

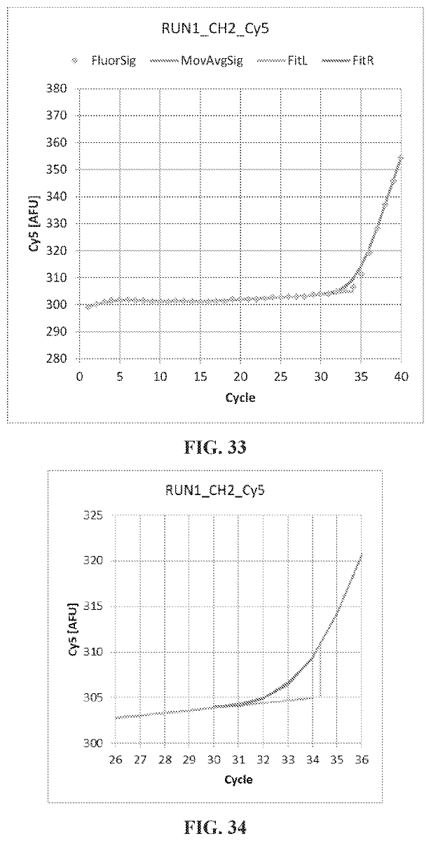

An example of Cy5 fluorescence readings of a positive SARS-CoV-2 specimen are depicted in FIG. 33. FitL and FitR are depicted. FIG. 34 depicts the region of FIG. 33 surrounding the breakpoint. The value of Cq is calculated where the difference between FitR and FitL is 1% (shown in orange).

In some embodiments, the normalization of the fluorescence signals using the methods described herein cancels out differences in optical gain. LED optical gain is the power of the light exciting fluorescence in the PCR chamber divided by the electrical power driving the LED. This is influenced by variability in properties of the LED, bandpass filter, and projections lens; as well as variability in how they're assembled into to the fluorimeter. Detector optical gain is the intensity of fluorescence being emitted from the PCR chamber divided by the electrical signal generated by the solid state detector. It is influenced by variability in properties of the two lenses, bandpass filter, and solid state detector, as well as variability in their placement during assembly. The methods described herein allow for analysis and comparison of amplification results irrespective of the LED optical gain or other variability in the instruments used.

The methods described herein find use with the devices (e.g., cartridges), instruments (e.g., with complimentary components for cartridge handling), and systems described herein, but also independently find use with other devices and systems. The components described herein (e.g., wax seals, fluorimeters, microfluidics, transfer capsule, etc.) find use in the devices (e.g., cartridges), instruments (e.g., with complimentary components for cartridge handling), and systems herein, as well as in performing the methods described herein, but also independently find use with other methods, devices, and systems.

### EXPERIMENTAL

### Example 1

### Exemplary Sample Preparation and Nucleic Analysis Protocol #1

350 μl of a liquid biological sample (e.g., a liquid sample, a specimen suspended in a buffer, etc.) are combined with a lyophilized lysis/capture reagent, allowing resuspension of the lysis/probe reagents in the liquid biological sample. The lyophilized lysis/capture reagent contains (1) reagents for cell lysis and digestion of cellular components (e.g., SDS, proteinase K, etc.), and (2) nucleic acid hybridization probes (i.e., sequence-specific probes) tethered to a biotin capture moiety that will allow for subsequent capture of nucleic acids hybridized to the probes. The biological sample and the lysis/capture reagents are incubated together for about 60 seconds as the temperature ramps to 90-100° C. to facilitate cell lysis. The suspension of cell lysate is then mixed and incubated at 68° C. to allow binding of the probes to target sequences within the sample nucleic acid. 260 μl of the probe-bound nucleic acid solution is combined with lyophilized streptavidin-coated magnetic beads. The resulting suspension is mixed at 75° C. to allow solubilization of the beads, and then the temperature is reduced to 68° C. to facilitate capture of the probe-bound nucleic acid onto the beads, through the binding of biotin on the probes to streptavidin on the beads. The resulting suspension is mixed well. A magnetic field is then applied to a single location within the suspension, thereby isolating the beads and any probes/target nucleic acids bound thereto into a pellet within the suspension. The pellet and the supernatant liquid are separated from one another by either (1) placing the magnetic field outside of the liquid, resulting in the beads being dragged or streamed across the liquid/air interface, or (2) removing the liquid while maintaining the magnetic field, such that the liquid is removed and the pellet remains in place. 300 μl of wash buffer is added to the pellet and the beads are resuspended with mixing. The process of forming a pellet and isolating the beads from the liquid is repeated. The beads are then resuspended in 300 μl of resuspension buffer. The suspension comprising the washed beads is then combined with a lyophilized amplification/detection reagent comprising fluorescently-labeled primers and nucleotides. Thermal cycling is then applied to the sample to amplify the target nucleic acid with the amplification reagents and the amplified target nucleic acid is detected.

Alternative protocols utilizing different reagents, different order of steps, different volumes, different temperatures, concentrated liquid reagents instead of dried reagents, etc., as discussed throughout, are within the scope of the embodiment herein.

### Example 2

### Exemplary Sample Preparation and Nucleic Analysis Protocol #2

350 μl of a liquid biological sample (e.g., a liquid sample, a specimen suspended in a buffer, etc.) are combined with a lyophilized lysis/digestion reagent, allowing resuspension of the lysis/digestion reagents in the liquid biological sample. The lyophilized lysis/digestion reagent contains reagents for cell lysis and digestion of cellular components (e.g., SDS, proteinase K, etc.). The biological sample and the lysis/digestion reagents are incubated together for about 60 seconds as the temperature ramps to 90-100° C. to facilitate cell lysis. 300-340 μl of the resulting cell lysate is combined with a lyophilized capture reagent which contains nucleic acid hybridization probes (i.e., sequence-specific probes) tethered to a biotin capture moiety that will allow for subsequent capture of nucleic acids hybridized to the probes. The suspension of cell lysate and capture probes is mixed and incubated at 68° C. 260 μl of the probe-bound nucleic acid solution is combined with lyophilized streptavidin-coated magnetic beads. The resulting suspension is mixed at 75° C. to allow solubilization of the beads, and then the temperature is reduced to 68° C. to facilitate capture of the probe-bound nucleic acid onto the beads, through the binding of biotin on the probes to streptavidin on the beads. The resulting suspension is mixed well. A magnetic field is then applied to a single location within the suspension, thereby isolating the beads and any probes/target nucleic acids bound thereto into a pellet within the suspension. The pellet and the supernatant liquid are separated from one another by either (1) placing the magnetic field outside of the liquid, resulting in the beads being dragged or streamed across the liquid/air interface, or (2) removing the liquid while maintaining the magnetic field, such that the liquid is removed and the pellet remains in place. 300 μl of wash buffer is added to the pellet and the beads are resuspended with mixing. The process of forming a pellet and isolating the beads from the liquid is repeated. The beads are then resuspended in 300 μl of resuspension buffer. The suspension comprising the washed beads is then combined with a lyophilized amplification/detection reagent comprising fluorescently-labeled primers and nucleotides. Thermal cycling is then applied to the sample to amplify the target nucleic acid with the amplification reagents and the amplified target nucleic acid is detected

Alternative protocols utilizing different reagents, different order of steps, different volumes, different temperatures, concentrated liquid reagents instead of dried reagents, etc., as discussed throughout, are within the scope of the embodiment herein.

### Example 3

### Exemplary Cq Determination Using Variable Light Sources

Table 1 shows the variability of LED excitation light and label fluorescence detection. For each of the 4 channels, the optical gains if 10 different LEDs and detectors were rank ordered with the strongest in each channel ranked as 1. For example, in channel 1, the LED with the highest optical gain has serial number (SN) 1-004, while LED with the lowest optical gain is SN 1-006. The detector with the highest optical gain is SN 1-002, and the lowest gain is SN 1-005.

[symbol]

TABLE 1

| LED | | | Detector | | |
|---|---|---|---|---|---|
| LED | Tape | Rank | [symbol] | [symbol] | Rank |
| 1-001 | 6,164 | 5 | 1-001 | 6,301 | 2 |
| 1-002 | 6,525 | 2 | 1-002 | 7,046 | 1 |
| 1-003 | 5,237 | 3 | 1-003 | 6,074 | 3 |
| 1-004 | 5,927 | 1 | 1-004 | 6,232 | 6 |
| 1-005 | 5,245 | 4 | 1-005 | 5,871 | 10 |
| 1-006 | [symbol] | 10 | 1-006 | 6,476 | 4 |
| 1-007 | [symbol] | 3 | 1-007 | 6,446 | 6 |
| 1-008 | [symbol] | 7 | 1-008 | 6,236 | 7 |
| 1-009 | [symbol] | 5 | 1-009 | 6,454 | 5 |
| 1-010 | [symbol] | 6 | 1-010 | [symbol] | 3 |
| 2-001 | 1,806 | 7 | 2-001 | 1,748 | 3 |
| 2-002 | 1,833 | 1 | 2-002 | 1,716 | 7 |
| 2-003 | 1,424 | 8 | 2-003 | 1,731 | 5 |
| 2-004 | 1,745 | 3 | 2-004 | 1,630 | 10 |
| 2-005 | 1,702 | 4 | 2-005 | 1,627 | 1 |
| 2-006 | 1,346 | 9 | 2-006 | 1,734 | 4 |
| 2-007 | 1,276 | 10 | 2-007 | 1,730 | 6 |
| 2-008 | 1,649 | 8 | 2-008 | 1,752 | 2 |
| 2-009 | 1,756 | 2 | 2-009 | 1,639 | 8 |
| 2-010 | [symbol] | 9 | 2-010 | 1,674 | 9 |
| 3-001 | 1,470 | 1 | 3-001 | 1,418 | 6 |
| 3-002 | 1,368 | 3 | 3-002 | 1,429 | 5 |
| 3-003 | 1,405 | 2 | 3-003 | 1,449 | 2 |
| 3-004 | 1,264 | 9 | 3-004 | 1,406 | 8 |
| 3-005 | 1,322 | 5 | 3-005 | 1,417 | 7 |
| 3-006 | 1,313 | 7 | 3-006 | 1,352 | 10 |
| 3-007 | 1,309 | 9 | 3-007 | 1,468 | 1 |
| 3-008 | 1,344 | 4 | 3-008 | 1,423 | 4 |
| 3-009 | [symbol] | 5 | 3-009 | 1,386 | 3 |
| 3-010 | 1,210 | 10 | 3-010 | 1,430 | 3 |
| 4-001 | [symbol] | 4 | 4-001 | 646 | 6 |
| 4-002 | [symbol] | 1 | 4-002 | 651 | 5 |
| 4-003 | [symbol] | 3 | 4-003 | 788 | 3 |
| 4-004 | [symbol] | 6 | 4-004 | 582 | 7 |
| 4-005 | [symbol] | 2 | 4-005 | 548 | 83 |
| 4-006 | 484 | 10 | 4-006 | 658 | 4 |
| 4-007 | 544 | 6 | 4-007 | 678 | 3 |
| 4-008 | 546 | 6 | 4-008 | 774 | 2 |
| 4-009 | [symbol] | 3 | 4-009 | 588 | 9 |
| 4-010 | [symbol] | 7 | 4-010 | 574 | 8 |

[symbol]

indicates data missing or illegible when filed

To determine the effect of absolute signal strength on Cq determination for both the process control (FAM) and SARS-CoV-2 (Cy5), the highest gain LED and detector were paired, and compared to the lowest LED-detector pair. The high FAM LED-detector pair were placed in channel 1 position, with the low pair in channel 3 position. The high Cy5 LED-detector pair were placed in channel 2 position, with the low pair in channel 4 position. Cq determinations using the methods described herein for a range of target concentrations (copies per reaction) are shown in Table 2.

TABLE 2

| Run | Copies | F1 | F3 | F3 − F4 | C2 | C4 | C2 − C4 |
|---|---|---|---|---|---|---|---|
| 1 | 1000 | 27.7 | 28.0 | −0.25 | 27.7 | 27.6 | 0.08 |
| 2 | 1000 | 27.8 | 28.1 | −0.24 | 27.5 | 27.5 | 0.03 |
| 3 | 1000 | 27.6 | 27.9 | −0.30 | 27.6 | 27.8 | −0.17 |
| | Mean/SD | 27.7 | 28.0 | 0.03 | 27.6 | 27.6 | 0.13 |
| 4 | 50 | 32.4 | 32.7 | −0.31 | 31.3 | 31.5 | −0.23 |
| 5 | 50 | 32.4 | 32.3 | 0.10 | 32.1 | 32.1 | 0.06 |
| 6 | 50 | 32.2 | 31.8 | 0.41 | 32.1 | 31.9 | 0.13 |
| | Mean/SD | 32.3 | 32.3 | 0.36 | 31.8 | 31.8 | 0.19 |
| 7 | 5 | 36.6 | 35.6 | 0.99 | Neg | Neg | |
| 8 | 5 | 36.5 | 36.1 | 0.42 | 34.4 | 34.9 | −0.51 |
| 9 | 5 | Neg | Neg | | 35.9 | 35.4 | 0.46 |
| 10 | 10 | 35.3 | 35.2 | 0.10 | 33.3 | 33.4 | −0.09 |
| 11 | 10 | Neg | Neg | | 34.6 | 34.9 | −0.33 |

For 1,000 copies, the means of three FAM reactions were 27.6 and 28.0, and 27.6 for both Cy5 reactions. At 50 copies, the means of the FAM reactions were both 32.3, and the Cy5 reactions were both 31.8. For the very low concentrations, 5 and 10 copies, results were similar when Cq was detected.

1. A cartridge for analyte detection comprising:
a storage section including a storage chamber;
a processing section including a processing chamber;
a microfluids section in fluid communication with the processing section; and a transfer capsule configured to transfer fluid between the storage chamber and the processing chamber.

2. The cartridge of claim 1, wherein the processing section is positioned between the storage section and the microfluidic section.

3. The cartridge of claim 1 or 2, further including a docking section with a access port in fluid communication with the storage chamber and a processing access port in fluid communication with the processing chamber.

4. The cartridge of one of claims 1-3, further including a body that forms at least a portion of the storage section, at least a portion of the processing section, and at least a portion of the docking section.

5. The cartridge of one of claims 1-3, wherein a first channel fluidly connects the storage access port and the storage chamber, and a second channel fluidly connects the processing access port and the processing chamber.

6. The cartridge of one of claims 1-5, wherein the storage chamber includes a first end and a second end opposite the first end, the first end is positioned closer to the storage access port than the second end, and wherein the first channel connects to the storage chamber at the second end.

7. The cartridge of one of claims 1-5, wherein the processing chamber includes a first end and second end opposite the first end, the first end is positioned closer to the processing access port than the second end, and wherein the second channel connects to the processing chamber at the second end.

8. The cartridge of one of claims 1-7, wherein the storage chamber is a first storage chamber and the storage section further includes a second storage chamber.

9. The cartridge of one of claims 1-8, wherein the processing chamber is a first processing chamber and the processing section further includes a second processing chamber.

10. The cartridge of one of claims 1-9, wherein the storage section includes a cavity configured to receive the transfer capsule.

11. The cartridge of one of claims 1-10, further including a first vent fluidly coupled to the storage chamber, and a second vent fluidly coupled to the processing chamber.

12. The cartridge of one of claims 1-11, wherein the microfluids section includes a reaction chamber, a microfluidic vent channel fluidly connected to the reaction chamber, and a microfluidic inlet channel fluidly connecting the processing chamber and the reaction chamber.

13. The cartridge of claim 12, wherein the microfluids section further includes a wax seal.

14. The cartridge of claim 13, wherein the wax seal is a first wax seal and the microfluids section further includes a second wax seal, wherein the first wax seal is positioned adjacent the microfluidic inlet channel and the second wax seal is positioned adjacent the microfluidic vent channel.

15. The cartridge of claim 14, wherein the second wax seal is positioned a distance from the reaction chamber, wherein the distance is at least 2 mm.

16. The cartridge of claim 12, further including an offset vent channel fluidly connected to the microfluidic inlet channel.

17. The cartridge of claim 16, wherein the offset vent channel is a first offset vent channel and the cartridge further includes a second offset vent channel fluidly connecting the first offset vent channel and the second vent.

18. A microfluidic device comprising:
(i) a reaction chamber,
(ii) an inlet channel in fluid communication with the reaction chamber;
(iii) a vent channel in fluid communication with the reaction chamber;
(iv) a first wax seal positioned adjacent to and in fluid communication with the inlet channel, wherein when first the wax seal is in a first position it does not occlude the inlet channel and allows fluid to enter the reaction chamber through the inlet channel, and wherein when first the wax seal is in a second position the first wax seal occludes the inlet channel and prevents fluid from entering or escaping the reaction chamber through the inlet channel;
(v) a second wax seal positioned adjacent to and in fluid communication with the vent channel, wherein when second the wax seal is in a first position it does not occlude the vent channel and allows gas to exit the reaction chamber through the vent channel, and wherein when second the wax seal is in a second position the second wax seal occludes the vent channel and prevents fluid from entering or escaping the reaction chamber through the vent channel;
wherein liquid reagents can be introduced to the reaction chamber through the inlet channel; and
wherein heating the wax seals above a threshold temperature melts the wax seals and subsequently cooling the wax seals below the threshold temperature solidifies the wax seals in the second position.

19. A system comprising a cartridge of one of claims 1-17 and an instrument into which the cartridge can be inserted, wherein the instrument comprises components to impart heating, magnetic transfer, fluid transfer, and/or analyte detection functionalities onto the cartridge.

20. Use of a system of claim 19 for sample processing and analyte detection.

21. A microfluidic system comprising an inlet channel, a vent channel, and a reaction chamber; wherein the inlet channel is in fluid communication with the reaction chamber, wherein the vent channel is in fluid communication with the reaction chamber;
the system further comprising a heating element capable of raising the temperature of the reaction chamber;
the system further comprising a first seal positioned adjacent to and in fluid communication with the inlet channel, wherein when first the seal is in a first position it does not occlude the inlet channel and allows fluid to enter the reaction chamber through the inlet channel, and wherein when the first seal is in a second position the first seal occludes the inlet channel and prevents fluid from entering or escaping the reaction chamber through the inlet channel;
the system further comprising a second seal positioned adjacent to and in fluid communication with the vent channel, wherein when the second seal is in a first position it does not occlude the vent channel and allows gas to exit the reaction chamber through the vent channel, and wherein when the second seal is in a second position the second seal occludes the vent channel and prevents fluid from entering or escaping the reaction chamber through the vent channel; and
wherein heating the seals above a threshold temperature melts the seals and allows the seals to flow from the first positions into the second positions, and wherein subsequently cooling the seals below the threshold temperature solidifies the wax seals in the second position.

22. The system of claim 21 wherein the first seal and the second seal comprise a wax or polymeric material.

23. A heat transfer device for heating and cooling a reaction chamber, the heat transfer device comprising:
a heat reservoir including a base, a first bore, a second bore, and a heat exchanger extending from the base; the heat exchanger includes a planar surface configured to about the reaction chamber;
a heater positioned within the first bore; and
a temperature sensor positioned within the second bore.

24. The heat transfer device of claim 23, wherein the first bore and the second bore are formed in the base.

25. The heat transfer device of claim 23, wherein the heat exchanger is cylindrical.

26. The heat transfer device of claim 23, wherein the heat reservoir is Aluminum.

27. The heat transfer device of claim 23, wherein the heater is an electric resistive heater.

28. The heat transfer device of claim 23, wherein the reaction chamber is a PCR reaction chamber.

29. The heat transfer device of claim 23, further including a processor and a non-transitory memory including instructions that when executed by the processor performs closed-loop temperature control of the heat reservoir.

30. An assembly comprising:
a first support;
a second support movable with respect to the first support;
a first heat transfer device with a first planar surface, the first heat transfer device coupled to the first support;
a second heat transfer device with a second planar surface positioned opposite the first planar surface, the second heat transfer device coupled to the second support; wherein the first heat transfer device and the second heat transfer device are at a first temperature;
a third heat transfer device with a third planar surface, the third heat transfer device coupled to the first support;
a fourth heat transfer device with a fourth planar surface positioned opposite the third planar surface, the fourth heat transfer device coupled to the second support; wherein the third heat transfer device and the fourth heat transfer device are at a second temperature different than the first temperature.

31. The assembly of claim 30, further including a fluorimeter coupled to the first support and positioned between the first heat transfer device and the third heat transfer device.

32. The assembly of claim 31, further including a fifth heat transfer device with a fifth planar surface position opposite the fluorimeter, the fifth heat transfer device coupled to the second support and positioned between the second heat transfer device and the fourth heat transfer device.

33. The assembly of claim 30, wherein the assembly is configured to receive a reaction chamber between the first planar surface and the second planar surface to bring the reaction chamber to the first temperature; and between the third planar surface and the fourth planar surface to bring the reaction chamber to the second temperature.

34. The assembly of claim 33, wherein the reaction chamber is a PCR chamber.

35. The assembly of claim 33, further comprising an actuator coupled to the second support and configured to move the second support along a clamp axis between a first position in which the first planar surface and the second planar surface are spaced apart by a first distance, and a second position in which the first planar surface and the second planar surface are spaced apart by a second distance, smaller than the first distance.

36. The assembly claim 35, Wherein the second distance is within a range of 400 micrometers to 600 micrometers.

37. The assembly of claim 33, wherein the actuator is a first actuator, and the assembly further includes a second actuator coupled to the first support and the second support, wherein the second actuator is configured to move the first support and the second support together along a translation axis.

38. The assembly of claim 37, wherein the translation axis is normal to the clamp axis.

39. The assembly of claim 30, wherein the first temperature is within a range of 80 degrees C. and 100 degrees C.

40. The assembly of claim 30, wherein the second temperature is within a range of 50 degrees C. and 70 degrees C.

41. A fluidic device comprising:
a reaction chamber;
a channel in fluid communication with the reaction chamber; and
a wax seal, wherein when the wax seal is in a first position it does not occlude the channel and allows fluid to enter or exit the reaction chamber through the channel, and when the wax seal is in a second position the wax seal occludes the channel and prevents fluid from entering or escaping the reaction chamber through the channel;
wherein heating the wax seal above a threshold temperature melts the wax seal and subsequently cooling the wax seal below the threshold temperature solidifies the wax seals in the second position.

42. The fluidic device of claim 41, wherein the reaction chamber is in fluid communication with an inlet channel; and the fluidic device further includes a vent channel in fluid communication with the reaction chamber.

43. The fluidic device of claim 42, wherein the inlet channel wax seal is a first wax seal and the fluidic device further includes a second wax seal, wherein when second the wax seal is in a first position it does not occlude the vent channel and allows fluid to exit the reaction chamber through the vent channel, and when the second wax seal is in a second position the second wax seal occludes the vent channel and prevents fluid from entering or escaping the reaction chamber through the vent channel.

44. The fluidic device of claim 43, wherein the first wax seal is positioned adjacent the inlet channel and the second wax seal is positioned adjacent the vent channel.

45. The fluidic device of claim 41, further comprising a first high-temperature movable heater capable of being positioned within a range of the wax seal to heat the wax seal above the threshold temperature.

46. The fluidic device of claim 45, further comprising a second low-temperature movable heater capable of being positioned within a range of the wax seal to cool the wax seal below the threshold temperature.

47. The fluidic device of claim 46, wherein the diameter of the wax seal is less than the diameter of the first and second heaters.

48. The fluidic device of claim 41, wherein the wax seal is coated in an adhesive.

49. The fluidic device of claim 48, wherein the adhesive is an acrylic adhesive.

50. The fluidic device of claim 41, wherein the first and second heaters are capable of being positioned at selected positions relative to the wax seal.

51. The fluidic device of claim 41, wherein the second wax seal is positioned a distance from the reaction chamber, wherein the distance is at least 2 mm.

52. The fluidic device of claim 42, wherein a plurality of liquid reagents can be introduced to the reaction chamber through the inlet channel.

53. A fluorimeter comprising:
a casing including a measurement aperture;
a first light source coupled to the casing along a first light source axis;
a second light source coupled to the casing along a second light source axis;
a third light source coupled to the casing along a third light source axis;
a fourth light source coupled to the casing along a fourth light source axis;
a first light detector coupled to the casing along a first detector axis;
a second light detector coupled to the casing along a second detector axis;
a third light detector coupled to the casing along a third detector axis;
a fourth light detector coupled to the casing along a fourth detector axis;
wherein the first light source axis, the second light source axis, the third light source axis, the fourth light source axis, the first detector axis, the second detector axis, the third detector axis, and the fourth detector axis intersect the measurement aperture.

54. The fluorimeter of claim 53, wherein the circular measurement aperture defines a normal axis through its center and perpendicular to the plane of the measurement aperture.

55. The fluorimeter of claim 54, wherein the normal axis, the first light source axis, the second light source axis, the third light source axis, the fourth light source axis, the first detector axis, the second detector axis, the third detector axis, and the fourth detector axis are not co-axial.

56. The fluorimeter of claim 54, the first light source axis, the second light source axis, the third light source axis, the fourth light source axis, the first detector axis, the second detector axis, the third detector axis, and the fourth detector axis are positioned circumferentially around the normal axis.

57. The fluorimeter of claim 53, wherein the first light source is positioned circumferentially adjacent to the first detector.

58. The fluorimeter of claim 53, wherein the fluorimeter does not include a dichroic mirror or a beam splitter.

59. The fluorimeter of claim 53, further comprising a processor and a non-transitory memory including instructions that, when executed by the processor, store 400 analog to digital readings by the first detector made over a 100 millisecond time period.

60. The fluorimeter of claim 53, wherein the first light source emits a first excitation light along the first light source axis; and wherein the first excitation light is reflected at the measurement aperture away from the first light detector axis.

61. The fluorimeter of claim 53, wherein the measurement aperture is configured to receive a sample; and wherein the first excitation light from the first light source has a first spectrum and the first light detector measures a first fluorescence of the sample in response to the first excitation light.

62. The fluorimeter of claim 61, wherein a second excitation light from the second light source has a second spectrum and the second light detector measures a second fluorescence of the sample in response to the second excitation light.

63. The fluorimeter of claim 53, wherein the measurement aperture is configured to align with a planar surface of a PCR chamber.

64. The fluorimeter of claim 53, wherein the first detector includes a first lens, a filter, a second lens, and a solid-state detector.

65. A nucleic acid quantification method comprising:
(a) performing a multicycle amplification reaction on a sample suspected of containing a target nucleic acid in the presence of a detectable reporter to produce an amplification product;
(b) detecting a signal from the detectable reporter that correlates with the amount of detectable reporter incorporated into the amplification product after each cycle of the amplification reaction;
(c) identifying earliest cycle with a normalized increase in signal that is greater than a cutoff value;
(d) fit a linear equation to a plurality of signals from cycles earlier than the earliest cycle with a normalized increase in signal that is greater than the threshold value;
(e) fit a curve to a plurality of signals from cycles later than the earliest cycle with a normalized increase in signal that is greater than the threshold value;
(f) identify the cycle (Cq) for which the normalized difference in signal for the linear equation and the curve is equal to a threshold value;
wherein Cq is inversely proportional to the amount of target nucleic acid present in the sample.

66. The method of claim 65, wherein step (c) comprises:
(i) identifying the cycle with the maximum normalized increase in signal;
(ii) if the maximum normalized increase in signal is greater than the cutoff value, then determine the earliest cycle prior to the cycle with the maximum normalized increase in signal that has a normalized increase in signal that is greater than a lower cutoff value.

67. The method of claim 65, further comprising a step of calculating a moving average of the detected signal for each cycle of the amplification reaction and using the moving averages for each cycle for steps (c)-(f).

68. The method of claim 67, wherein the moving average is calculated as the average of the signal at each cycle with signals at the immediate two earlier and immediate two later cycles.

69. The method of claim 65, wherein the curve is a quadratic curve.

70. The method of claim 65, wherein the multicycle amplification reaction is a 30-50 cycle amplification reaction.

71. The method of claim 70, wherein the multicycle amplification reaction is a 40 cycle amplification reaction.

72. The method of claim 65, wherein the multicycle amplification reaction is quantitative polymerase chain reaction (qPCR).

73. The method of claim 65, wherein the detectable reporter is a fluorophore and the signal is fluorescence.

74. The method of claim 65, wherein each cycle comprises a nucleic acid denaturation step, an annealing/extension step, and a detection step.

75. The method of claim 65, wherein each cycle comprises a nucleic acid denaturation step, an annealing step, an extension step, and a detection step.

76. The method of claim 65, wherein the sample is a biological sample.

77. The method of claim 76, wherein the target nucleic acid is a viral nucleic acid.

78. The method of claim 77, wherein the amount of target nucleic acid present in the sample is proportional to the viral load in the sample.

79. A method of preparing a target nucleic acids in a biological sample for subsequent analysis, comprising:
(a) combining the biological sample with a lysis reagent capable of digesting cell membranes and degrading proteins and allowing the lysis reagent to digest cell membranes and degrade proteins to generate a lysate, wherein the biological sample comprises nucleic acid;
(b) combining the lysate with a capture reagent, wherein the capture reagent comprises a nucleic acid probe tethered to a capture moiety;
(c) allowing the nucleic acid probe to hybridize to the nucleic acids of the biological sample to generate a probe-bound nucleic acid solution;
(d) combining the probe-bound nucleic acid with capture-agent-coated magnetic beads;
(e) allowing the capture agent to bind to the capture moiety to generate a bead-captured nucleic acid suspension;
(f) isolating bead-captured nucleic acids within the bead-captured nucleic acid suspension by exposing a portion of the bead-captured nucleic acid suspension to a magnetic field; and
(g) separating the isolated, bead-captured nucleic acids from a liquid portion of the bead-captured nucleic acid suspension.

80. A method of preparing a target nucleic acids in a biological sample for subsequent analysis, comprising:
(a) combining the biological sample with a lysis reagent and a capture reagent, wherein the lysis reagent comprises components capable of digesting cell membranes and degrading cellular proteins, wherein the capture reagent comprises a nucleic acid probe tethered to a capture moiety, and wherein the biological sample comprises nucleic acid;
(b) allowing the lysis reagent to digest cell membranes and degrade proteins to generate a lysate;
(c) allowing the nucleic acid probe to hybridize to the nucleic acids of the biological sample to generate a probe-bound nucleic acid solution;
(d) combining the probe-bound nucleic acid with capture-agent-coated magnetic beads;
(e) allowing the capture agent to bind to the capture moiety to generate a bead-captured nucleic acid suspension;
(f) isolating bead-captured nucleic acids within the bead-captured nucleic acid suspension by exposing a portion of the bead-captured nucleic acid suspension to a magnetic field; and
(g) separating the isolated, bead-captured nucleic acids from a liquid portion of the bead-captured nucleic acid suspension.

81. The method of claim 79 or 80, wherein the lysis reagent is a dry lysis reagent, and wherein the dry lysis reagent is resuspended in the biological sample.

82. The method of claim 79 or 80, wherein the lysis reagent is a concentrated liquid lysis reagent, and wherein the concentrated liquid lysis reagent is diluted in the biological sample.

83. The method of claim 79 or 80, wherein the capture reagent is a dry capture reagent, and wherein the dry capture reagent is resuspended in the lysate.

84. The method of claim 79 or 80, wherein the capture reagent is a concentrated liquid capture reagent, and wherein the concentrated liquid capture reagent is diluted in the lysate.

85. The method of claim 79 or 80, wherein the capture-agent-coated magnetic beads are dry and are resuspended in the probe-bound nucleic acid solution.

86. The method of claim 79 or 80, wherein the capture-agent-coated magnetic beads are in a concentrated liquid and are diluted in the probe-bound nucleic acid solution.

87. The method of claim 79 or 80, wherein the method does not comprise a centrifugation step.

88. The method of claim 79 or 80, wherein the method does not comprise a filtration step.

89. The method of claim 79 or 80, wherein the method does not comprise precipitation of nucleic acids.

90. The method of claim 79 or 80, wherein the nucleic acids are not isolated from contaminants within the biological sample in steps (a) through (e).

91. The method of claim 79 or 80, wherein allowing the lysis reagent to digest cell membranes and degrade proteins to generate a lysate comprises mixing of the biological sample and the lysis reagent as the temperature is raised to 90-100° C.

92. The method of claim 79 or 80, wherein the lysis reagent comprises a protease capable of digesting cellular proteins.

93. The method of claim 92, wherein the protease is proteinase K.

94. The method of claim 79 or 80, wherein the lysis reagent comprises a detergent at a concentration sufficient to lyse cells.

95. The method of claim 94, wherein the detergent is SDS.

96. The method of claim 79 or 80, wherein the lysis reagent comprises one or more salts.

97. The method of claim 79 or 80, wherein the capture moiety is biotin and the capture agent is streptavidin.

98. The method of claim 79 or 80, wherein the nucleic acid probe comprises a hybridization sequence that is complementary to a target sequence in the nucleic acids of the biological sample.

99. The method of claim 98, wherein allowing the nucleic acid probe to hybridize to the nucleic acids of the biological sample comprises mixing the lysate and the capture reagent.

100. The method of claim 98, wherein allowing the nucleic acid probe to hybridize to the nucleic acids of the biological sample comprises incubating the lysate and the capture reagent at 63-73° C.

101. The method of claim 100, wherein allowing the nucleic acid probe to hybridize to the nucleic acids of the biological sample comprises incubating the lysate and the capture reagent at about 68° C.

102. The method of claim 98, wherein the lysate and the capture reagent are mixed and/or incubated for 30 seconds to 5 minutes.

103. The method of claim 102, wherein the lysate and the capture reagent are mixed and/or incubated for 1 to 3 minutes.

104. The method of claim 103, wherein the lysate and the capture reagent are incubated for about 2 minutes.

105. The method of claim 79 or 80, wherein combining the probe-bound nucleic acid with the capture-agent-coated magnetic beads comprises mixing the probe-bound nucleic acid and the capture-agent-coated magnetic beads

106. The method of claim 79 or 80, wherein combining the probe-bound nucleic acid with the capture-agent-coated magnetic beads comprises incubating the probe-bound nucleic acid and the capture-agent-coated magnetic beads at 70-80° C.

107. The method of claim 106, wherein combining the probe-bound nucleic acid with the capture-agent-coated magnetic beads comprises incubating the probe-bound nucleic acid and the capture-agent-coated magnetic beads at about 75° C.

108. The method of claim 79 or 80, wherein allowing the capture agent to bind to the capture moiety comprises incubating the probe-bound nucleic acid and the capture-agent-coated magnetic beads at 63-73° C.

109. The method of claim 108, wherein allowing the capture agent to bind to the capture moiety comprises incubating the probe-bound nucleic acid and the capture-agent-coated magnetic beads at about 68° C.

110. The method of claim 79 or 80, further comprising aspirating the bead-captured nucleic acid suspension prior to exposure to the magnetic field

111. The method of claim 79 or 80, wherein separating the isolated, bead-captured nucleic acids and the liquid portion of the bead-captured nucleic acid suspension comprises holding the magnetic field in place and removing the liquid portion from the bead-captured nucleic acid suspension;

112. The method of claim 79 or 80, further comprising:
(h) combining the isolated bead-captured nucleic acid with a wash buffer;
(i) resuspending the bead-captured nucleic acid in the wash buffer;
(j) isolating the bead-captured nucleic acids within the wash buffer by exposing a portion of the bead-captured nucleic acids to a magnetic field; and
(k) separating the isolated, bead-captured nucleic acids from the wash buffer.

113. The method of claim 112, further comprising repeating steps (h)-(k) one or more times.

114. The method of claim 79 or 80, further comprising:
(h) combining the isolated bead-captured nucleic acid with a resuspension buffer; and
(i) resuspending the bead-captured nucleic acid in the resuspension buffer to generate a bead-captured nucleic acid resuspension.

115. The method of claim 114, further comprising:
(j) combining the bead-captured nucleic acid resuspension with analysis reagents.

116. The method of claim 112, further comprising:
(l) combining the isolated bead-captured nucleic acid with a resuspension buffer; and
(m) resuspending the bead-captured nucleic acid in the resuspension buffer to generate a bead-captured nucleic acid resuspension.

117. The method of claim 116, further comprising:
(n) combining the bead-captured nucleic acid resuspension with analysis reagents.

118. The method of claim 115 or 117, wherein the analysis reagents are dry analysis reagents and combining the bead-captured nucleic acid resuspension with the dry analysis reagents comprises resuspending the dry analysis reagents in the bead-captured nucleic acid resuspension.

119. The method of claim 115 or 117, wherein the analysis reagents comprise detection reagents.

120. The method of claim 115 or 117, wherein the analysis reagents comprise amplification reagents.

121. The method of claim 115 or 117, further comprising amplifying and/or detecting the target nucleic acid hybridized to the bead-bound capture reagent.

122. The method of claim 78 or 80, wherein the wash buffer and resuspension buffer contain the same components.

123. A method of preparing a target nucleic acids in a biological sample for subsequent analysis, comprising:
(a) combining the biological sample comprising cells with a dry lysis reagent comprising proteinase K, SDS, and salt; and resuspending the dry lysis reagent in the biological sample to generate a lysate;
(b) combining the lysate with a dry capture reagent and resuspending the dry capture reagent in the lysate, wherein the capture reagent comprises a nucleic acid probe tethered to a capture moiety, wherein the capture moiety is biotin, and wherein the nucleic acid probe comprises a hybridization sequence that is complementary to a target sequence within the nucleic acids of the biological sample;
(c) incubating the nucleic acid probe and the nucleic acids of the lysate at a temperature of 63-73° C. to generate a probe-bound nucleic acid solution;
(d) combining the probe-bound nucleic acid with dry capture-agent-coated magnetic beads and resuspending the dry capture-agent-coated magnetic beads in the probe-bound nucleic acid at 70-80° C., wherein the capture agent is streptavidin;
(e) incubating the probe-bound nucleic acid and capture-agent-coated magnetic beads at 63-73° C. and allowing the capture agent to bind to the capture moiety to generate a bead-captured nucleic acid suspension;
(f) isolating bead-captured nucleic acids within the bead-captured nucleic acid suspension by exposing a portion of the bead-captured nucleic acid suspension to a magnetic field, and (A) holding the magnetic field in place while removing the liquid portion from the bead-captured nucleic acid suspension, or (B) moving the magnetic field to drag the bead-captured nucleic acids from the liquid portion; and
(g) separating the isolated, bead-captured nucleic acids from a liquid portion of the bead-captured nucleic acid suspension;
(h) combining the isolated bead-captured nucleic acid with a wash buffer;
(i) resuspending the bead-captured nucleic acid in the wash buffer;
(j) isolating the bead-captured nucleic acids within the wash buffer by exposing a portion of the bead-captured nucleic acids to a magnetic field; and
(k) separating the isolated, bead-captured nucleic acids from the wash buffer;
(l) combining the isolated bead-captured nucleic acid with a resuspension buffer; and
(m) resuspending the bead-captured nucleic acid in the resuspension buffer to generate a bead-captured nucleic acid resuspension;
(n) combining the bead-captured nucleic acid resuspension with dry analysis reagents and resuspending the dry analysis reagents in the bead-captured nucleic acid resuspension, wherein the analysis reagents comprise primers and detectable labels for amplifying and detecting the target nucleic acids; and
(o) amplifying and detecting the target nucleic acid hybridized to the bead-bound capture reagent;
wherein the method does not comprise a centrifugation step, a filtration step, or nucleic acid precipitation step; wherein the nucleic acids are not isolated from contaminants within the biological sample in steps (a) through (e); and wherein the wash buffer and resuspension buffer contain the same components.

124. A method of preparing a target nucleic acids in a biological sample for subsequent analysis, comprising:
(a) combining the biological sample with a dry reagent comprising lysis/digestion components and capture components, and resuspending the dry reagent in the biological sample, wherein the lysis/digestion components comprise proteinase K, SDS, and salt, wherein the capture components comprise comprises a nucleic acid probe tethered to a capture moiety, wherein the capture moiety is biotin, and wherein the nucleic acid probe comprises a hybridization sequence that is complementary to a target sequence within the nucleic acids of the biological sample
(b) incubating biological sample at a temperature 90-100° C. to allow lysis of cell membranes and digestion of proteins within the sample to generate a lysate;
(c) incubating the lysate at a temperature of 63-73° C. to allow binding of the hybridization sequence of the nucleic acid probe to the target sequence of the nucleic acid of the biological sample to generate a probe-bound nucleic acid solution;
(d) combining the probe-bound nucleic acid with dry capture-agent-coated magnetic beads and resuspending the dry capture-agent-coated magnetic beads in the probe-bound nucleic acid at 70-80° C., wherein the capture agent is streptavidin;
(e) incubating the probe-bound nucleic acid and capture-agent-coated magnetic beads at 63-73° C. and allowing the capture agent to bind to the capture moiety to generate a bead-captured nucleic acid suspension;
(f) isolating bead-captured nucleic acids within the bead-captured nucleic acid suspension by exposing a portion of the bead-captured nucleic acid suspension to a magnetic field, and (A) holding the magnetic field in place while removing the liquid portion from the bead-captured nucleic acid suspension, or (B) moving the magnetic field to drag the bead-captured nucleic acids from the liquid portion; and
(g) separating the isolated, bead-captured nucleic acids from a liquid portion of the bead-captured nucleic acid suspension;
(h) combining the isolated bead-captured nucleic acid with a wash buffer;
(i) resuspending the bead-captured nucleic acid in the wash buffer;
(j) isolating the bead-captured nucleic acids within the wash buffer by exposing a portion of the bead-captured nucleic acids to a magnetic field; and
(k) separating the isolated, bead-captured nucleic acids from the wash buffer; (1) combining the isolated bead-captured nucleic acid with a resuspension buffer; and
(m) resuspending the bead-captured nucleic acid in the resuspension buffer to generate a bead-captured nucleic acid resuspension;
(n) combining the bead-captured nucleic acid resuspension with dry analysis reagents and resuspending the dry analysis reagents in the bead-captured nucleic acid resuspension, wherein the analysis reagents comprise primers and detectable labels for amplifying and detecting the target nucleic acids; and
(o) amplifying and detecting the target nucleic acid hybridized to the bead-bound capture reagent;
wherein the method does not comprise a centrifugation step, a filtration step, or nucleic acid precipitation step; wherein the nucleic acids are not isolated from contaminants within the biological sample in steps (a) through (e); and wherein the wash buffer and resuspension buffer contain the same components.

125. The method of one of claims 79-124, wherein the method is performed manually.

126. The method of claim 125, wherein movement and combining of liquids is conducted by manual pipetting.

127. The method of one of claims 79-124, wherein the method is automated.

128. The method of claim 127, wherein the method steps are performed within a single-use cartridge.

129. The method of claim 127, wherein the single-use cartridge contains all dry and liquid reagents and buffers for performing the method steps of the method.

130. The method of claim 128, wherein the single use cartridge interfaces with an instrument that comprises components for combining and mixing reagents, heating elements, and a magnet.

131. The method of claim 127, wherein the method steps are performed by an automation instrument within one or more tubes or wells.

132. A system or kit comprising:
(a) a lysis reagent capable of digesting cell membranes and degrading cellular proteins;
(b) a capture reagent comprising a nucleic acid probe tethered to a capture moiety;
(c) capture-agent-coated magnetic beads;
(d) amplification/detection reagents; and
(e) a wash/resuspension buffer solution.

133. A system or kit comprising:
(a) a lysis/capture reagent comprising (1) lysis/digestion components capable of digesting cell membranes and degrading proteins, and (2) a nucleic acid probe tethered to a capture moiety;
(b capture-agent-coated magnetic beads;
(c) amplification/detection reagents; and
(d) a wash/resuspension buffer solution.

134. The system or kit of claim 132 or 133, wherein the regents are in a dry or concentrated liquid form.

135. The system or kit of claim 132 or 133, further comprising a biological sample comprising cells.

136. The system or kit of claim 132 or 133, wherein the lysis reagent or lysis/digestion components comprises a protease capable of digesting cellular proteins.

137. The system or kit of claim 136, wherein the protease is proteinase K.

138. The system or kit of claim 132 or 133, wherein the lysis reagent or lysis/digestion components comprises a detergent in an amount sufficient to lyse cells when combined with a biological sample.

139. The system or kit of claim 138, wherein the detergent is SDS.

140. The system or kit of claim 132 or 133, wherein the lysis reagent or lysis/digestion components comprises one or more salts.

141. The system or kit of claim 132 or 133, wherein the capture moiety is biotin and the capture agent is streptavidin.

142. The system or kit of claim 132 or 133, wherein the nucleic acid probe comprises a hybridization sequence that is complementary to a target sequence in a target nucleic acid.

143. The system or kit of claim 132 or 133, wherein the dry amplification/detection reagents comprises primers, detectable labels, and nucleotides.

144. A system or kit comprising:
(a) a lysis reagent comprising proteinase K, SDS, and one or more salts;
(b) a capture reagent comprising a nucleic acid probe tethered to a biotin capture moiety;
(c) capture-agent-coated magnetic beads, wherein the capture agent is streptavidin;
(d) amplification/detection reagents comprising primers, detectable labels, and nucleotides; and
(e) a buffer solution.

145. A system or kit comprising:
(a) a lysis/capture reagent comprising (1) lysis/digestion components comprising proteinase K, SDS, and one or more salts, and (2) a nucleic acid probe tethered to a capture moiety;
(b) capture-agent-coated magnetic beads, wherein the capture agent is streptavidin;
(c) amplification/detection reagents comprising primers, detectable labels, and nucleotides; and
(d) a buffer solution.

146. The system or kit of claim 144 or 145, wherein the regents are in a dry or concentrated liquid form.

147. The system or kit of claim 144 or 145, further comprising a biological sample comprising cells.

148. The system or kit of claim 132, 133, 144 or 145, further comprising disposable laboratory products for manually using the system or kit for the capture, isolation, amplification, and detection of a target nucleic acid from a biological sample comprising cells.

149. The system or kit of claim 148, wherein the disposable laboratory products comprise pipette tips, reaction tubes, and or a microwell plate.

150. The system or kit of claim 149, further comprising a single-use cartridge containing the components of (a)-(e), wherein the single-use cartridge is capable of interfacing with an instrument that comprises components for combining and mixing reagents, heating elements, and a magnet.
